SEQUENCE LISTING (31 sequences)

SEQ ID NO: 1 - DNA, 2163 bp, Homo sapiens
  CDS (1)..(2160)
atgagctttatttgcaggccacgaggagggggcaggctggagacagattccagggtggcagcaggggggtggacagcgggaagccatacagtgggcaaagagcaaaaggcctcagatacgtcacccatgggccacagagagcaaggagccagcataggagacggaggagaaacagctggagagggaggagagcggccaagtgtaaggtctgggagtggagatgtggagcaggggcttggggtctgcggctgcagcaaccacaccctctgggctgggagggccaagggcagccggggccctcctgtaactccacccatggccctgcctgcagacttcctcatgcacaagctgacggcctccgacacggggaagacctgcctgatgaaggccttgttaaacatcaaccccaacaccaaggagatagtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgcccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgcgtttttgcttggatttggagtagccttggcctcgctgatcgagaagtgtcccaaagacaacaaggactgcagctcctacggcagcttcagcgacgcagtgctggaactcttcaagctcaccataggcctgggtgayctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaatggaagacgcacgtctccttccttaacgaagacccggggcctgtaagacgaacaggtactgtcgctgtgaggtga

SEQ ID NO: 2 - protein, 720 aa, Homo sapiens
MSFICRPRGGGRLETDSRVAAGGWTAGSHTVGKEQKASDTSPMGHREQGASIGDGGETAGEGGERPSVRSGSGDVEQGLGVCGCSNHTLWAGRAKGSRGPPVTPPMALPADFLMHKLTASDTGKTCLMKALLNINPNTKEIVRILLAFAEENDILGRFINAEYTEEAYEGQTALNIAIERRQGDIAALLIAAGADVNAHAKGAFFNPKYQHEGFYFGETPLALAACTNQPEIVQLLMEHEQTDITSRDSRGNNILHALVTVAEDFKTQNDFVKRMYDMILLRSGNWELETTRNNDGLTPLQLAAKMGKAEILKYILSREIKEKRLRSLSRKFTDWAYGPVSSSLYDLTNVDTTTDNSVLEITVYNTNIDNRHEMLTLEPLHTLLHMKWKKFAKHMFFLSFCFYFFYNITLTLVSYYRPREEEAIPHPLALTHKMGWLQLLGRMFVLIWAMCISVKEGIAIFLLRPSDLQSILSDAWFHFVFFIQAVLVILSVFLYLFAYKEYLACLVLAMALGWANMLYYTRGFQSMGMYSVMIQKVILHDVLKFLFVYIAFLLGFGVALASLIEKCPKDNKDCSSYGSFSDAVLELFKLTIGLGDLNIQQNSKYPILFLFLLITYVILTFVLLLNMLIALMGETVENVSKESERIWRLQRARTILEFEKMLPEWLRSRFRMGELCKVAEDDFRLCLRINEVKWTEWKTHVSFLNEDPGPVRRTGTVAVR

SEQ ID NO: 3 - DNA, 2238 bp, Homo sapiens
  CDS (1)..(2235)
atgagctttatttgcaggccacgaggagggggcaggctggagacagattccagggtggcagcaggggggtggacagcgggaagccatacagtgggcaaagagcaaaaggcctcagatacgtcacccatgggccacagagagcaaggagccagcataggagacggaggagaaacagctggagagggaggagagcggccaagtgtaaggtctgggagtggagatgtggagcaggggcttggggtctgcggctgcagcaaccacaccctctgggctgggagggccaagggcagccggggccctcctgtaactccacccatggccctgcctgcagacttcctcatgcacaagctgacggcctccgacacggggaagacctgcctgatgaaggccttgttaaacatcaaccccaacaccaaggagatagtgcggatcctgcttgcctttgctgaagagaacgacatcctgggcaggttcatcaacgccgagtacacagaggaggcctatgaagggcagacggcgctgaacatcgccatcgagcggcggcagggggacatcgcagccctgctcatcgccgccggcgccgacgtcaacgcgcacgccaagggggccttcttcaaccccaagtaccaacacgaaggcttctacttcggtgagacgcccctggccctggcagcatgcaccaaccagcccgagattgtgcagctgctgatggagcacgagcagacggacatcacctcgcgggactcacgaggcaacaacatccttcacgccctggtgaccgtggccgaggacttcaagacgcagaatgactttgtgaagcgcatgtacgacatgatcctactgcggagtggcaactgggagctggagaccactcgcaacaacgatggcctcacgccgctgcagctggccgccaagatgggcaaggcggagatcctgaagtacatcctcagtcgtgagatcaaggagaagcggctccggagcctgtccaggaagttcaccgactgggcgtacggacccgtgtcatcctccctctacgacctcaccaacgtggacaccaccacggacaactcagtgctggaaatcactgtctacaacaccaacatcgacaaccggcatgagatgctgaccctggagccgctgcacacgctgctgcatatgaagtggaagaagtttgccaagcacatgttctttctgtccttctgcttttatttcttctacaacatcaccctgaccctcgtctcgtactaccgaccccgggaggaggaggccatcccgcaccccttggccctgacgcacaagatggggtggctgcagctcctagggaggatgtttgtgctcatctgggccatgtgcatctctgtgaaagagggcattgccatcttcctgctgagaccctcggatctgcagtccatcctctcggatgcctggttccactttgtcttttttatccaagctgtgcttgtgatactgtctgtcttcttgtacttgtttgcctacaaagagtacctcgcctgcctcgtgctggccatggccctgggctgggcgaacatgctctactatacgcggggtttccagtccatgggcatgtacagcgtcatgatccagaaggtcattttgcatgatgttctgaagttcttgtttgtatatatcgcgtttttgcttggatttggagtagccttggcctcgctgatcgagaagtgtcccaaagacaacaaggactgcagctcctacggcagcttcagcgacgcagtgctggaactcttcaagctcaccataggcctgggtgayctgaacatccagcagaactccaagtatcccattctctttctgttcctgctcatcacctatgtcatcctcacctttgttctcctcctcaacatgctcattgctctgatgggcgagactgtggagaacgtctccaaggagagcgaacgcatctggcgcctgcagagagccaggaccatcttggagtttgagaaaatgttaccagaatggctgaggagcagattccggatgggagagctgtgcaaagtggccgaggatgatttccgactgtgtttgcggatcaatgaggtgaagtggactgaatggaagacgcacgtctccttccttaacgaagacccggggcctgtaagacgaacagatttcaacaaaatccaagattcttccaggaacaacagcaaaaccactctcaatgcatttgaagaagtcgaggaattcccggaaacctcggtgtag

SEQ ID NO: 4 - protein, 745 aa, Homo sapiens
MSFICRPRGGGRLETDSRVAAGGWTAGSHTVGKEQKASDTSPMGHREQGASIGDGGETAGEGGERPSVRSGSGDVEQGLGVCGCSNHTLWAGRAKGSRGPPVTPPMALPADFLMHKLTASDTGKTCLMKALLNINPNTKEIVRILLAFAEENDILGRFINAEYTEEAYEGQTALNIAIERRQGDIAALLIAAGADVNAHAKGAFFNPKYQHEGFYFGETPLALAACTNQPEIVQLLMEHEQTDITSRDSRGNNILHALVTVAEDFKTQNDFVKRMYDMILLRSGNWELETTRNNDGLTPLQLAAKMGKAEILKYILSREIKEKRLRSLSRKFTDWAYGPVSSSLYDLTNVDTTTDNSVLEITVYNTNIDNRHEMLTLEPLHTLLHMKWKKFAKHMFFLSFCFYFFYNITLTLVSYYRPREEEAIPHPLALTHKMGWLQLLGRMFVLIWAMCISVKEGIAIFLLRPSDLQSILSDAWFHFVFFIQAVLVILSVFLYLFAYKEYLACLVLAMALGWANMLYYTRGFQSMGMYSVMIQKVILHDVLKFLFVYIAFLLGFGVALASLIEKCPKDNKDCSSYGSFSDAVLELFKLTIGLGDLNIQQNSKYPILFLFLLITYVILTFVLLLNMLIALMGETVENVSKESERIWRLQRARTILEFEKMLPEWLRSRFRMGELCKVAEDDFRLCLRINEVKWTEWKTHVSFLNEDPGPVRRTDFNKIQDSSRNNSKTTLNAFEEVEEFPETSV

SEQ ID NO: 5 - protein, 839 aa, Homo sapiens [sequence omitted]

SEQ ID NO: 6 - protein, 764 aa, Homo sapiens
MTSPSSSPVFRLETLDGGQEDGSEADRGKLDFGSGLPPMESQFQGEDRKFAPQIRVNLNYRKGTGASQPDPNRFDRDRLFNAVSRGVPEDLAGLPEYLSKTSKYLTDSEYTEGSTGKTCLMKAVLNLKDGVNACILPLLQIDRDSGNPQPLVNAQCTDDYYRGHSALHIAIEKRSLQCVKLLVENGANVHARACGRFFQKGQGTCFYFGELPLSLAACTKQWDVVSYLLENPHQPASLQATDSQGNTVLHALVMISDNSAENIALVTSMYDGLLQAGARLCPTVQLEDIRNLQDLTPLKLAAKEGKIEIFRHILQREFSGLSHLSRKFTEWCYGPVRVSLYDLASVDSCEENSVLEIIAFHCKSPHRHRMVVLEPLNKLLQAKWDLLIPKFFLNFLCNLIYMFIFTAVAYHQPTLKKQAAPHLKAEVGNSMLLTGHILILLGGIYLLVGQLWYFWRRHVFIWISFIDSYFEILFLFQALLTVVSQVLCFLAIEWYLPLLVSALVLGWLNLLYYTRGFQHTGIYSVMIQKVILRDLLRFLLIYLVFLFGFAVALVSLSQEAWRPEAPTGPNATESVQPMEGQEDEGNGAQYRGILEASLELFKFTIGMGELAFQEQLHFRGMVLLLLLAYVLLTYILLLNMLIALMSETVNSVATDSWSIWKLQKAISVLEMENGYWWCRKKQRAGVMLTVGTKPDGSPDERWCFRVEEVNWASWEQTLPTLCEDPSGAGVPRTLENPVLASPPKEDEDGASEENYVPVQLLQSN

SEQ ID NO: 7 - protein, 871 aa, Homo sapiens
MADSSEGPRAGPGEVAELPGDESGTPGGEAFPLSSLANLFEGEDGSLSPSPADASRPAGPGDGRPNLRMKFQGAFRKGVPNPIDLLESTLYESSVVPGPKKAPMDSLFDYGTYRHHSSDNKRWRKKIIEKQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYSSLYDLSSLDTCGEEASVLEILVYNSKIENRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRDRWSSVVPRVVELNKNSNPDEVVVPLDSMGNPRCDGHQQGYPRKWRTEDAPL

SEQ ID NO: 8 - protein, 725 aa, Homo sapiens
MGLSLPKEKGLILCLWSKFCRWFQRRESWAQSRDEQNLLQQKRIWESPLLLAAKDNDVQALNKLLKYEDCKVHQRGAMGETALHIAALYDNLEAAMVLMEAAPELVFEPMTSELYEGQTALHIAVVNQNMNLVRALLARRASVSARATGTAFRRSPCNLIYFGEHPLSFAACVNSEEIVRLLIEHGADIRAQDSLGNTVLHILILQPNKTFACQMYNLLLSYDRHGDHLQPLDLVPNHQGLTPFKLAGVEGNTVMFQHLMQKRKHTQWTYGPLTSTLYDLTEIDSSGDEQSLLELIITTKKREARQILDQTPVKELVSLKWKRYGRPYFCMLGAIYLLYIICFTMCCIYRPLKPRTNNRTSPRDNTLLQQKLLQEAYMTPKDDIRLVGELVTVIGAIIILLVEVPDIFRMGVTRFFGQTILGGPFHVLIITYAFMVLVTMVMRLISASGEVVPMSFALVLGWCNVMYFARGFQMLGPFTIMIQKMIFGDLMRFCWLMAVVILGFASAFYIIFQTEDPEELGHFYDYPMALFSTFELFLTIIDGPANYNVDLPFMYSITYAAFAIIATLLMLNLLIAMMGDTHWRVAHERDELWRAQIVATTVMLERKLPRCLWPRSGICGREYGLGDRWFLRVEDRQDLNRQRIQRYAQAFHTRGSEDLDKDSVEKLELGCPFSPHLSLPMPSVSRSTSRSSANWERLRQGTLRRDLRGIINRGLEDGESWEYQI

SEQ ID NO: 9 - protein, 21 aa, Homo sapiens
MFFLSFCFYFFYNITLTLVSY

SEQ ID NO: 10 - protein, 25 aa, Homo sapiens
LLGRMFVLIWAMCISVKEGIAIFLL

SEQ ID NO: 11 - protein, 21 aa, Homo sapiens
FVFFIQAVLVILSVFLYLFAY

SEQ ID NO: 12 - protein, 19 aa, Homo sapiens
YLACLVLAMALGWANMLYY

SEQ ID NO: 13 - protein, 20 aa, Homo sapiens
FLFVYIAFLLGFGVALASLI

SEQ ID NO: 14 - protein, 19 aa, Homo sapiens
ILFLFLLITYVILTFVLLL

SEQ ID NO: 15 - protein, 23 aa, Homo sapiens
YRPREEEAIPHPLALTHKMGWLQ

SEQ ID NO: 16 - protein, 15 aa, Homo sapiens
RPSDLQSILSDAWFH

SEQ ID NO: 17 - protein, 40 aa, Homo sapiens
TRGFQSMGMYSVMIQKVILHDVLKFLFVYIAFLLGFGVAL

SEQ ID NO: 18 - protein, 42 aa, Homo sapiens
EKCPKDNKDCSSYGSFSDAVLELFKLTIGLGDLNIQQNSKYP

SEQ ID NO: 19 - DNA, 20 bp, Homo sapiens
cgcagtgctggaactcttca

SEQ ID NO: 20 - DNA, 23 bp, Homo sapiens
catcagagcaatgagcatgttga

SEQ ID NO: 21 - DNA, 24 bp, Homo sapiens
gcccaggatgtcgttctcttcagc

SEQ ID NO: 22 - DNA, 26 bp, Homo sapiens
gatccgcactatctccttggtgttgg

SEQ ID NO: 23 - DNA, 27 bp, Homo sapiens
actgaatggaagacgcacgtctccttc

SEQ ID NO: 24 - DNA, 20 bp, Homo sapiens
tgacctgaacatccagcaga

SEQ ID NO: 25 - DNA, 21 bp, Homo sapiens
agcatgttgaggaggagaaca

SEQ ID NO: 26 - DNA, 20 bp, Homo sapiens
cggaaacctcggtgtagaag

SEQ ID NO: 27 - DNA, 20 bp, Homo sapiens
tcatccctcaaagcctctct

SEQ ID NO: 28 - DNA, 38 bp, Homo sapiens
gcagcagcggccgccacatgttctttctgtccttctgc

SEQ ID NO: 29 - DNA, 36 bp, Homo sapiens
gcagcagtcgaccctcacagcgacagtacctgttcg

SEQ ID NO: 30 - DNA, 39 bp, Homo sapiens
gcagcagcggccgcatgagctttatttgcaggccacgag

SEQ ID NO: 31 - DNA, 37 bp, Homo sapiens
gcagcagtcgacgttgaggaggagaacaaaggtgagg

(19) United States
(12) Patent Application Publication
(10) Pub. No.: US 2003/0027164 A1
(43) Pub. Date: Feb. 6, 2003

(54) Novel human nucleic acid molecules and polypeptides encoding a novel human ion channel expressed in spinal cord and brain

(76) Inventors: Glen T. Gaughan, Northford, CT (US); John N. Feder, Belle Mead, NJ (US); Thomas C. Nelson, Lawrenceville, NJ (US); Gabe Mintier, Hightstown, NJ (US); Chandra S. Ramanathan, Wallingford, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/000,823

(22) Filed: Nov. 30, 2001

Related U.S. Application Data
(60) Provisional application No. 60/250,587, filed on Dec. 1, 2000.

(51) Int. Cl. C12Q 1/68; C07H 21/04; C12P 21/02; C12N 5/06; C07K 14/705

(52) U.S. Cl. 435/6; 435/69.1; 435/320.1; 435/325; 536/23.5

(57) ABSTRACT

The present invention relates to novel human nucleic acid molecules encoding novel human cation channels, and proteins and polypeptides encoded by such nucleic acid molecules. More specifically, the nucleic acid molecules of the invention include novel human genes, e.g., hVR1d.1 and hVR1d.2, that encode proteins or polypeptides that are expressed in spinal cord and brain tissues and display sequence homology and structural homology to the vanilloid and TRP (transient receptor potential) families of cation channel proteins. The proteins and polypeptides of the invention directed to this novel human cation channel may be therapeutically valuable targets for drug delivery in the treatment of human diseases that involve calcium, sodium, potassium or other ionic homeostatic dysfunction, such as central nervous system (CNS) disorders, e.g., degenerative neurological disorders such as Alzheimer's disease or Parkinson's disease, or other disorders such as chronic pain, anxiety and depression, stroke, cardiac disorders, e.g., arrhythmia, diabetes, hypercalcemia, hypocalcemia, hypercalciuria, hypocalciuria, or ion disorders associated with immunological disorders, gastro-intestinal (GI) tract disorders or renal or liver disease.

This application claims benefit to provisional application U.S. Serial No. 60/250,587, filed Dec. 1, 2000.

### 1. INTRODUCTION

The present invention relates to the isolation and identification of novel human nucleic acid molecules and proteins and polypeptides encoded by such nucleic acid molecules, or degenerate variants thereof, encoding novel human ion channels. More specifically, the nucleic acid molecules of the invention relate to a novel human gene, termed hVR1d, that encodes proteins or polypeptides that are expressed in spinal cord and brain tissues and display sequence homology and structural homology to the 20 vanilloid and TRP (transient receptor potential) families of cation channel proteins. The proteins and polypeptides of the invention directed to this novel human cation channel may be therapeutically valuable targets for drug delivery in the treatment of human diseases that involve calcium, sodium, potassium or other ionic homeostatic dysfunction, such as central nervous system (CNS) disorders, e.g., degenerative neurological disorders such as Alzheimer's disease or Parkinson's disease, or other disorders such as chronic pain, anxiety and depression, stroke, cardiac disorders, e.g., arrhythmia, diabetes, hypercalcemia, hypocalcemia, hypercalciuria, hypocalciuria, or ion disorders associated with immunological disorders, gastrointestinal (GI) tract disorders or renal or liver disease.

### 2. BACKGROUND OF THE INVENTION

Control of the internal ionic environment is an extremely important function of all living cells. Ion exchange with the external medium is regulated by a variety of means, the most important of which are various transporters and ion channels. Ion channels comprise a very large and diverse family of proteins which play an important role in cell homeostasis, hormone and neurotransmitter release, motility, neuronal action potential generation and propagation and other vital intra- and inter-cellular functions. Thus, these channels are important targets for the development of therapeutic compounds in the treatment of disease. A number of proteins have been described as forming ion channels, including the vanilloid and TRP protein families. These proteins have been shown to function as cation channels of varying degrees of selectivity and with different, and in some cases unknown, mechanisms for channel gating. For example, the TRP family of ion channels comprises a group of proteins some of which are believed to form store-operated calcium (Ca²⁺) channels, i.e., ion channels that operate to allow the influx of extracellular Ca²⁺ into cells when the intracellular stores of calcium are depleted (Zhu et al., 1996, Cell 85: 661-671). It is believed that TRP ion channels are expressed, in some form, in most, if not all, animal tissues (Zhu et al., supra at 661). In addition, another protein, termed trp-like or trpl, has been disclosed (Phillips et al., 1992, Neuron 8: 631-642; Gillo et al., 1996, PNAS USA 93: 14146-14151) and it has been suggested that there may be a cooperative interaction between TRP and TRPL proteins, perhaps these proteins contributing channel subunits to form a multimeric Ca²⁺ channel (Gillo et al., supra).

The capsaicin receptor, also known as VR1 or vanilloid receptor subtype 1, has been isolated from rats and characterized as a Ca²⁺-permeable non-selective ion channel that is structurally related to the TRP family of ion channels (Caterina et al., 1997, Nature 389: 816-824). The rat VR1 cDNA contains an open reading frame of 2,514 nucleotides encoding a 838-amino acid protein. Hydrophilicity studies have indicated that VR1 contains six transmembrane domains with a short hydrophobic stretch between transmembrane regions 5 and 6 which may represent the ion permeation path. In addition, VR1 is disclosed as containing three ankyrin repeat domains at the N-terminal end of the protein (Caterina et al., supra at 820). It has been noted that VR1 resembles the trp and trpl proteins in topological organization, the presence of multiple N-terminal ankyrin repeats and in amino acid sequence homology within and adjacent to the sixth transmembrane domain (Caterina et al, supra at 820-821). However, outside of these regions of homology, there is actually very little sequence homology between VR1 and the TRP-related proteins. Moreover, studies have indicated that VR1 is not a store-operated Ca²⁺ channel as are some of the TRP proteins and the expression of this protein is restricted to sensory neurons (Caterina et al., supra at 821 and FIG. 6 at 820; Mezey, E. et al., 2000, Proc. Natl. Acad. Sci. USA 97: 3655-3660).

Human VR1 (also known in the art as “hVR1” or “OTRPC1”) has been disclosed in PCT Patent Application WO 99/37675 and PCT Patent Application WO 00/29577, which disclose nucleotide and amino acid sequences for human VR1 as well as another subtype, human VR2 (also known in the art as “hVR2”, “VANILREP2”, “VRRP”, “VLR” or “OTRPC2”). In addition, PCT Patent Application WO 99/37765 discloses nucleotide and amino acid sequences for VANILREP2 and polymorphic variants thereof. The VANILREP2 protein sequence set forth in PCT application WO 99/37765 appears to be essentially the same as hVR2 disclosed in PCT application WO 99/37675. See also PCT Application WO 99/46377, which corresponds to EP 953638 A1, PCT application WO 00/22121, and GB patent application 2346882 A, which also disclose the nucleotide and amino acid sequences for hVR2.

Additional members of the vanilloid family of cation channels have also been identified. For example, a homologue of VR1, termed SIC, was cloned from the rat kidney. This protein was identified as a stretch-inactivating channel (SIC), i.e., it is inactivated by membrane stretch, and as being expressed mainly in the kidney and liver. SIC was further described as sharing the same transmembrane and pore alignments with VR1 but having different electrophysiological properties (Suzuki et al., March 1999, J. Biol. Chem. 274 (No. 10): 6330-6335). Recent reports, however, indicate that SIC may be a chimera of VR1 and a newly-identified VR subtype, OTRPC4 (see, e.g., Strotmann et al., October 2000, Nature Cell Biology 2: 695-702 and Liedtke W. et al., 2000, Cell 103: 525-535). Moreover, it has been noted in the art that, despite structural homologies between members of the vanilloid family, respective proteins within the family may possess significant differences, e.g., in conductance or permeability to various ions (Suzuki et al., supra at 6335).

Another cation channel protein that has been identified as sharing a relatively low sequence homology (<30%) with the vanilloid family is ECaC (epithelial calcium channel). This protein was initially cloned from rabbit kidney cells and found to be expressed in the proximal small intestine, the kidney and the placenta of the rabbit. This protein was disclosed as resembling the VR1 and TRP family of receptors in predicted topological organization and the presence of multiple NH₂-terminal ankyrin repeats. In addition, amino acid sequence homologies between ECaC, VR1 and the TRP-related proteins were noted within and adjacent to the sixth transmembrane segment, including the predicted region for the ion permeation path (Hoenderop et al., March 1999, J. Biol. Chem. 274 (No. 13): 8375-8378). However, it was also noted that, despite these structural and sequence homologies, there is actually a low sequence homology between these proteins outside of the sixth transmembrane segment, “suggesting a distant evolutionary relationship among these channels.” (Hoenderop et al., supra at 8377).

More recently, the human homologue of ECaC, hECaC, has been identified and disclosed as having a <30% sequence homology with other Ca²⁺ channels and as being highly expressed in kidney, small intestine, and pancreas (see Muller, et al., 2000, Genomics 67: 48-53).

Yet another Ca²⁺ transport protein, CaT1, has been identified from rat duodenum, which protein is structurally related to the ECaC, VR1, and TRP ion channels. However, CaT1 is not stimulated by capsaicin or calcium store depletion, as would be expected with VR1 and the TRP receptors, respectively, thus suggesting that CaT1 is not a subtype of the VR1 or TRP ion channels (Peng et al., August 1999, J. Biol. Chem. 274 (No. 32): 22739-22746). More recently, a homologue of CaT1, termed CaT2, has been identified in the rat (Peng et al., September 2000, J. Biol. Chem. 275 (36): 28186-28194).

Finally, it should be noted that, while the proteins described above have clear structural and sequence homologies (compare Zhu et al., supra, FIG. 6D at 668, Caterina et al., supra, FIG. 5b at 819, and Hoenderop et al., FIG. 1B at 8376), they nevertheless display varying patterns of tissue expression, electrophysiological properties and functions (e.g., selective vs. non-selective), such that it is acknowledged in the art that these molecules, while distantly related from an evolutionary standpoint, are a diverse group of proteins with significantly different and distinct properties and functions (Suzuki et al., supra at 6335; Hoenderop et al., supra at 8377; and Caterina et al., supra at 822). For a review of the various members 30 of this complex family of proteins, see Harteneck et al., 2000, Trends Neurosci. 23: 159-166.

### 3. SUMMARY OF THE INVENTION

The present invention relates to the isolation and identification of novel nucleic acid molecules and proteins and polypeptides encoded by such nucleic acid molecules, or degenerate variants thereof, that participate in the formation or function of novel human ion channels. More specifically, the nucleic acid molecules of the invention are directed to a novel human gene, termed “hVR1d”, that encodes proteins or polypeptides involved in the formation or function of a novel human cation channel. The novel hVR1d proteins of the invention display some sequence homology and structural homology to the TRP and vanilloid family of cation channels but represent distinct human channel proteins with distinct distribution patterns, e.g., tissue expression. The hVR1d proteins of the invention are highly expressed in spinal cord and brain tissues.

According to one embodiment of the invention, a novel human hVR1d cDNA and the amino acid sequence of its derived expressed protein is disclosed. This cDNA has been isolated in two splice forms, hVR1d.1 and hVR1d.2, which differ in the absence (hVR1d.1) or presence (hVR1d.2) of a short nucleotide segment at the 3′ end of the molecule. The encoded proteins corresponding to these hVR1d cDNAs show a modest level of homology to the human vanilloid receptor family of ion channels.

The compositions of this invention include nucleic acid molecules (also termed herein as “nucleic acids”), e.g., the hVR1d.1 and hVR1d.2 nucleic acid molecules, including recombinant DNA molecules, cloned genes or degenerate variants thereof, especially naturally occurring variants, that encode novel hVR1d.1 and hVR1d.2 gene products, and antibodies directed against such gene products or conserved variants or fragments thereof.

In particular, the compositions of the present invention include nucleic acid molecules (also referred to herein as “hVR1d nucleic acid molecules or nucleic acids”) that comprise the following sequences: (a) the nucleotide sequences of the human hVR1d.1 and hVR1d.2 splice variants as depicted in FIGS. 1A and 1B, respectively, as well as allelic variants and homologs thereof; (b) nucleotide sequences that encode the hVR1d.1 or hVR1d.2 gene product amino acid sequences as depicted in FIGS. 2A and 2B, respectively; (c) nucleotide sequences that encode portions of the hVR1d.1 or hVR1d.2 gene products corresponding to functional domains and individual exons; (d) nucleotide sequences comprising the novel hVR1d.1 or hVR1d.2 nucleic acid sequences disclosed herein, or portions thereof, that encode mutants of the corresponding gene product in which all or a part of one or more of the domains is deleted or altered; (e) nucleotide sequences that encode fusion proteins comprising the hVR1d.1 or hVR1d.2 gene product, or one or more of its domains, fused to a heterologous polypeptide; (f) nucleotide sequences within the hVR1d.1 or hVR1d.2 gene, as well as chromosome sequences flanking those genes, that can be utilized as part of the methods of the present invention for the diagnosis or treatment of human disease; and (g) nucleotide sequences that hybridize to the above-described sequences under highly or moderately stringent conditions. The nucleic acids of the invention include, but are not limited to, cDNA and genomic DNA molecules of the hVR1d.1 or hVR1d.2 genes.

The present invention also encompasses gene products of the nucleic acid molecules listed above; i.e., proteins and/or polypeptides that are encoded by the above-disclosed hVR1d nucleic acid molecules, e.g., the hVR1d.1 and hVR1d.2 nucleic acid molecules, and are expressed in recombinant host systems. In a preferred embodiment, the hVR1 proteins of the invention include the proteins encoded by the amino acid sequences of hVR1d.1 and hVR1d.2 as depicted in FIGS. 2A (SEQ ID NO:2) and 2B (SEQ ID NO:4), respectively, or functionally equivalent fragments or derivatives thereof. These proteins can be produced by recombinant means or by chemical synthesis methods known in the art.

Antagonists and agonists of the hVR1d genes and/or gene products disclosed herein are also included in the present invention. Such antagonists and agonists will include, for example, small molecules, large molecules, and antibodies directed against the hVR1d.1 or hVR1d.2 proteins and polypeptides of the invention. Antagonists and agonists of the invention also include nucleotide sequences, such as antisense and ribozyme molecules, and gene or regulatory sequence replacement constructs, that can be used to inhibit or enhance expression of the disclosed hVR1d nucleic acid molecules.

The present invention further encompasses cloning vectors, including expression vectors, that contain the nucleic acid molecules of the invention and can be used to express those nucleic acid molecules in host organisms. The present invention also relates to host cells engineered to contain and/or express the nucleic acid molecules of the invention. Further, host organisms that have been transformed with these nucleic acid molecules are also encompassed in the present invention, e.g., transgenic animals, particularly transgenic non-human animals, and particularly transgenic non-human mammals.

The present invention also relates to methods and compositions for the diagnosis of human disease involving cation, e.g., Ca²⁺, sodium or potassium channel, dysfunction or lack of other ionic homeostasis including but not limited to CNS disorders, e.g., degenerative neurological diseases such as Alzheimer's or Parkinson's disease, or other disorders such as chronic pain, anxiety and depression, cardiac disorders, e.g., arrhythmia, or other disorders such as diabetes, hypercalcemia, hypercalciuria, or ion disorders associated with immunological disorders, GI tract disorders or renal or liver disease. Such methods comprise, for example, measuring expression of the hVR1d gene in a patient sample, or detecting a mutation in the gene in the genome of a mammal, including a human, suspected of exhibiting ion channel dysfunction. The nucleic acid molecules of the invention can also be used as diagnostic hybridization probes or as primers for diagnostic PCR analysis to identify hVR1d gene mutations, allelic variations, or regulatory defects, such as defects in the expression of the gene. Such diagnostic PCR analyses can be used to diagnose individuals with disorders associated with a particular hVR1d gene mutation, allelic variation, or regulatory defect. Such diagnostic PCR analyses can also be used to identify individuals susceptible to ion channel disorders.

Methods and compositions, including pharmaceutical compositions, for the treatment of ion channel disorders are also included in the invention. Such methods and compositions are capable of modulating the level of hVR1d, e.g., hVR1d1.1 or hVR1d.2, gene expression and/or the level of activity of the respective gene product or polypeptide. Such methods include, for example, modulating the expression of the hVR1d gene and/or the activity of the hVR1d gene product for the treatment of a disorder that is mediated by a defect in some other gene.

Such methods also include screening methods for the identification of compounds that modulate the expression of the nucleic acids and/or the activity of the polypeptides of the invention, e.g., assays that measure hVR1d mRNA and/or gene product levels, or assays that measure levels of hVR1d activity, such as the ability of the gene products to allow Ca²⁺ influx into cells.

For example, cellular and non-cellular assays are known that can be used to identify compounds that interact with the hVR1d gene and/or gene product, e.g., modulate the activity of the gene and/or bind to the gene product. Such cell-based assays of the invention utilize cells, cell lines, or engineered cells or cell lines that express the gene product.

In one embodiment, such methods comprise contacting a compound to a cell that expresses a hVR1d gene, measuring the level of gene expression, gene product expression, or gene product activity, and comparing this level to the level of the hVR1d gene expression, gene product expression, or gene product activity produced by the cell in the absence of the compound, such that if the level obtained in the presence of the compound differs from that obtained in its absence, a compound that modulates the expression of the hVR1d gene and/or the synthesis or activity of the gene product has been identified.

In an alternative embodiment, such methods comprise administering a compound to a host organism, e.g., a transgenic animal that expresses a hVR1d transgene or a mutant hVR1d transgene, and measuring the level of hVR1d gene expression, gene product expression, or gene product activity. The measured level is compared to the level of hVR1d gene expression, gene product expression, or gene product activity in a host that is not exposed to the compound, such that if the level obtained when the host is exposed to the compound differs from that obtained when the host is not exposed to the compound, a compound that modulates the expression of the hVR1d gene and/or the synthesis or activity of hVR1d gene products has been identified.

The compounds identified by these methods include therapeutic compounds that can be used as pharmaceutical compositions to reduce or eliminate the symptoms of ion channel disorders such as CNS disorders, e.g., degenerative neurological diseases such as Alzheimer's or Parkinson's disease, or other disorders such as chronic pain, anxiety and depression, cardiac disorders, e.g., arrhythmia, or other disorders such as diabetes, hypercalcemia, hypercalciuria, or ion disorders associated with immunological disorders, GI tract disorders or renal or liver disease.

The present invention also relates to an isolated nucleic acid comprising a nucleic acid sequence that encodes a polypeptide having the amino acid sequence of FIG. 2A (SEQ ID NO:2) or FIG. 2B (SEQ ID NO:4), or the complement of the nucleic acid of said sequence(s) .

The present invention also relates to an isolated nucleic acid comprising a nucleic acid sequence capable of hybridizing under stringent conditions to a nucleic acid molecule of FIG. 1A (SEQ ID NO:1) or FIG. 1B (SEQ ID NO:3) and encoding a hVR1d polypeptide having an activity of a naturally-occurring hVR1d protein.

The present invention also relates to an isolated nucleic acid comprising the nucleic acid sequence of FIG. 1A (SEQ ID NO:1).

The present invention also relates to an isolated nucleic acid comprising the nucleic acid sequence of FIG. 1B (SEQ ID NO:3).

The present invention also relates to an isolated nucleic acid of FIG. 1A (SEQ ID NO:1) or FIG. 1B (SEQ ID NO:3), wherein the nucleic acid is genomic or cDNA.

The present invention also relates to an isolated nucleic acid of FIG. 1A (SEQ ID NO:1) or FIG. 1B (SEQ ID NO:3), which is RNA.

The present invention also relates to an isolated nucleic acid of FIG. 1A (SEQ ID NO:1) or FIG. 1B (SEQ ID NO:3), further comprising a label.

The present invention also relates to an isolated nucleic acid wherein to any nucleic acid described herein that encodes an hVR1d protein or polypeptide is linked in frame to a nucleic acid sequence that encodes a heterologous protein or peptide.

The present invention also relates to a nucleic acid comprising a nucleic acid sequence encoding (a) a deletion mutant of hVR1d.1; (b) a deletion mutant of hVR1d.2; or (c) the complement of the nucleic acid sequences of (a) or (b).

The invention further relates to an isolated nucleic acid molecule of SEQ ID NO:1, and/or 3, wherein the nucleotide sequence comprises sequential nucleotide deletions from either the C-terminus or the N-terminus.

The invention further relates to an isolated polypeptide molecule of SEQ ID NO:2, and/or 4, wherein the polypeptide sequence comprises sequential amino acid deletions from either the C-terminus or the N-terminus.

The invention further relates to a nucleic acid comprising a nucleic acid sequence encoding (a) an addition mutant of hVR1d.1; (b) an addition mutant of hVR1d.2; or (c) the complement of the nucleotide sequences of (a) or (b).

The invention further relates to a nucleic acid comprising a nucleic acid sequence encoding (a) a substitution mutant of hVR1d.1; (b) a substitution mutant of hVR1d.2; or (c) the complement of the nucleic acid sequences of (a) or (b).

The invention further relates to a recombinant vector comprising a nucleic acid of the present invention.

The invention further relates to an expression vector comprising a nucleic acid of the present invention operatively associated with a regulatory nucleotide sequence containing transcriptional and translational regulatory information that controls expression of the nucleic acid in a host cell.

The invention further relates to an expression vector comprising a nucleic acid of the present invention operatively associated with a regulatory nucleotide sequence containing transcriptional and translational regulatory information that controls expression of the nucleic acid in a host cell.

The invention further relates to a delivery complex comprising an expression vector described herein and a targeting means.

The invention further relates to a genetically engineered host cell containing a nucleic acid of the present invention

The invention further relates to an genetically engineered host cell containing a nucleic acid described herein operatively associated with a regulatory nucleotide sequence containing transcriptional and translational regulatory information that controls expression of the nucleic acid sequence in a host cell.

The invention further relates to an genetically engineered host cell containing a nucleic acid described herein operatively associated with a regulatory nucleotide sequence containing transcriptional and translational regulatory information that controls expression of the nucleic acid sequence in a host cell.

The invention further relates to a method of making an hVR1d polypeptide comprising the steps of (a) culturing a host cell in an appropriate culture medium to produce an hVR1d polypeptide; and (b) isolating the hVR1d polypeptide.

The invention further relates to a method of making an hVR1d polypeptide comprising the steps of: (a) culturing a genetically engineered host cell containing a nucleic acid described herein operatively associated with a regulatory nucleotide sequence containing transcriptional and translational regulatory information that controls expression of the nucleic acid sequence in a host cell in an appropriate culture medium to produce an hVR1d polypeptide; and (b) isolating the hVR1d polypeptide.

The invention further relates to a method of making an hVR1d polypeptide, wherein the hVR1d polypeptide is hVR1d1.1 or hVR1d.2 or a functionally equivalent derivative thereof.

The invention further relates to a method of antibody preparation which is specifically reactive with an epitope of an hVR1d polypeptide.

The invention further relates to a method of making a transgenic animal comprising a nucleic acid of the present invention.

The invention further relates to a substantially pure polypeptide encoded by a nucleic acid of the present invention.

The invention further relates to a substantially pure polypeptide encoded by the nucleic acid sequence provided in the deposited clone.

The invention further relates to a substantially pure human hVR1d polypeptide as depicted in FIGS. 2A (SEQ ID NO:2) or 2B (SEQ ID NO:4).

The invention further relates to a substantially pure polypeptide which is at least 90% identical to the polypeptide as set forth in FIGS. 2A (SEQ ID NO:2) or 2B (SEQ ID NO:4).

The invention further relates to a fusion protein comprising a polypeptide of the present invention and a second heterologous polypeptide.

The invention further relates to a pharmaceutical preparation comprising a therapeutically effective amount of the polypeptide of the present invention and a pharmaceutically acceptable carrier.

The invention further relates to a test kit for detecting and/or quantitating a wild type or mutant hVR1d nucleic acid molecule in a sample, comprising the steps of contacting the sample with a nucleic acid of the present invention; and detecting and/or quantitating the label as an indication of the presence or absence and/or amount of a wild type or mutant hVR1d nucleic acid.

The invention further relates to a test kit for detecting and/or quantitating a wild type or mutant hVR1d polypeptide in a sample, comprising the steps of contacting the sample with an antibody of the present invention; and detecting and/or quantitating a polypeptide-antibody complex as an indication of the presence or absence and/or amount of a wild type or mutant hVR1d nucleic acid.

The invention further relates to a method for identifying compounds that modulate hVR1d activity comprising: (a) contacting a test compound to a cell that expresses a hVR1d gene; (b) measuring the level of hVR1d gene expression in the cell; and (c) comparing the level obtained in (b) with the hVR1d gene expression obtained in the absence of the compound; such that if the level obtained in (b) differs from that obtained in the absence of the compound, a compound that modulates hVR1d activity is identified.

The invention further relates to a method for identifying compounds that modulate hVR1d activity comprising: (a) contacting a test compound to a cell that contains a hVR1d polypeptide; (b) measuring the level of hVR1d polypeptide or activity in the cell; and (c) comparing the level obtained in (b) with the level of hVR1d polypeptide or activity obtained in the absence of the compound; such that if the level obtained in (b) differs from that obtained in the absence of the compound, a compound that modulates hVR1d activity is identified.

The invention further relates to a method for identifying compounds that regulate ion channel-related disorders, comprising: (a) contacting a test compound with a cell which expresses a nucleic acid of the present invention and (b) determining whether the test compound modulates hVR1d activity.

The invention further relates to a method for identifying compounds that regulate ion channel-related disorders comprising: (a) contacting a test compound with a nucleic acid of the present invention; and (b) determining whether the test compound interacts with the nucleic acid of the present invention.

The invention further relates to a method for identifying compounds that regulate ion channel-related disorders, comprising: (a) contacting a test compound with a cell or cell lysate containing a reporter gene operatively associated with a hVR1d regulatory element; and (b) detecting expression of the reporter gene product.

The invention further relates to a method for identifying compounds that regulate ion channel-related disorders comprising: (a) contacting a test compound with a cell or cell lysate containing hVR1d transcripts; and (b) detecting the translation of the hVR1d transcript.

The invention further relates to a method for modulating ion channel-related disorders in a subject, comprising administering to the subject a therapeutically effective amount of a hVR1d polypeptide.

The invention further relates to a method for modulating ion channel-related disorders in a subject, wherein the hVR1d polypeptide is hVR1d.1 or hVR1d.2, or a functionally equivalent derivative thereof.

The invention further relates to a method for modulating ion channel-related disorders in a subject, wherein the hVR1d polypeptide is hVR1d.1 or hVR1d.2, or a functionally equivalent derivative thereof wherein the subject is a human.

The invention further relates to a method of gene therapy, comprising administering to a subject an effective amount of a delivery complex of the present invention.

The invention further relates to a method for the treatment of ion channel-related disorders, comprising modulating the activity of a hVR1d polypeptide.

The invention further relates to a method for the treatment of ion channel-related disorders, comprising modulating the activity of a hVR1d polypeptide, wherein the hVR1d polypeptide is hVR1d.1 or hVR1d.2, or a functionally equivalent derivative thereof.

The invention further relates to a method for the treatment of ion channel-related disorders, comprising modulating the activity of a hVR1d polypeptide, wherein the hVR1d polypeptide is hVR1d.1 or hVR1d.2, or a functionally equivalent derivative thereof, wherein the method comprises administering an effective amount of a compound that agonizes or antagonizes the activity of the hVR1d polypeptide.

The invention further relates to a method for the treatment of ion channel-related disorders, comprising administering an effective amount of a compound that decreases expression of a hVR1d gene.

The invention further relates to a method for the treatment of ion channel-related disorders, comprising administering an effective amount of a compound that decreases expression of a hVR1d gene in which the compound is an oligonucleotide encoding an antisense or ribozyme molecule that targets hVR1d transcripts and inhibits translation.

The invention further relates to a method for the treatment of ion channel-related disorders, comprising administering an effective amount of a compound that decreases expression of a hVR1d gene in which the compound is an oligonucleotide encoding an antisense or ribozyme molecule that targets hVR1d transcripts and inhibits translation, in which the compound is an oligonucleotide that forms a triple helix with the promoter of the hVR1d gene and inhibits transcription.

The invention further relates to a method for the treatment of ion channel-related disorders, comprising administering an effective amount of a compound that increases expression of a hVR1d gene.

The invention further relates to a pharmaceutical formulation for the treatment of ion channel-related disorders, comprising a compound that activates or inhibits hVR1d activity, mixed with a pharmaceutically acceptable carrier.

The invention further relates to a method of identifying a compound that modulates the biological activity of hVR1d, comprising the steps of, (a) combining a candidate modulator compound with hVR1d having the sequence set forth in one or more of SEQ ID NO:2 or SEQ ID NO:4; and measuring an effect of the candidate modulator compound on the activity of hVR1d.

The invention further relates to a method of identifying a compound that modulates the biological activity of an ion channel, comprising the steps of, (a) combining a candidate modulator compound with a host cell expressing hVR1d having the sequence as set forth in SEQ ID NO:2 or SEQ ID NO:4; and, (b) measuring an effect of the candidate modulator compound on the activity of the expressed hVR1d.

The invention further relates to a method of identifying a compound that modulates the biological activity of hVR1d, comprising the steps of, (a) combining a candidate modulator compound with a host cell containing a vector described herein, wherein hVR1d is expressed by the cell; and, (b) measuring an effect of the candidate modulator compound on the activity of the expressed hVR1d.

The invention further relates to a method of screening for a compound that is capable of modulating the biological activity of hVR1d, comprising the steps of: (a) providing a host cell described herein; (b) determining the biological activity of hVR1d in the absence of a modulator compound; (c) contacting the cell with the modulator compound; and (d) determining the biological activity of hVR1d in the presence of the modulator compound; wherein a difference between the activity of hVR1d in the presence of the modulator compound and in the absence of the modulator compound indicates a modulating effect of the compound.

The invention further relates to a compound that modulates the biological activity of human hVR1d as identified by the methods described herein.

### 4. DESCRIPTION OF THE FIGURES

FIGS. 1A and 1B. Human hVR1d.1 and hVR1d.2 nucleic acid sequences, respectively. The putative start codon is bolded and the stop codon is underlined.

FIGS. 2A and 2B. Human hVR1d.1 and hVR1d.2 amino acid sequences, respectively, with the six transmembrane domains in boldface, the ankyrin domains underscored and the pore loop region boxed.

FIG. 3. Alignment of amino acid sequences for hVR1d.2 with the reported vanilloid receptors hVR1, hVR2, OTRPC4, and hECaC (using GCG pileup program).

FIG. 4. Tissue expression profile of hVR1d.

FIG. 5. Tissue expression profile of the hVR1d splice variant, hVR1d.

FIG. 6. Tissue expression profile of the hVR1d splice variant, hVR1d.2, in brain subregions.

### 5. DETAILED DESCRIPTION OF THE INVENTION

The present invention relates to the isolation and identification of novel nucleic acid molecules, as well as novel proteins and polypeptides, for the formation or function of novel human ion channels. More specifically, the invention relates to a novel human gene, hVR1d, that includes two different splice variants, hVR1d.1 and hVR1d.2, that encode corresponding hVR1d.1 and hVR1d.2 proteins or biologically active derivatives or fragments thereof, involved in the formation or function of cation channels. All references to hVR1d shall also be construed to apply to hVR1d.1 and hVR1d.2 unless explicitly stated otherwise herein.

The hVR1d nucleic acid molecules of the present invention include isolated naturally-occurring or recombinantly-produced human hVR1d.1 and hVR1d.2 nucleic acid molecules, e.g., DNA molecules, cloned genes or degenerate variants thereof. The compositions of the invention also include isolated, naturally-occurring or recombinantly-produced human hVR1d.1 and hVR1d.2 proteins or polypeptides.

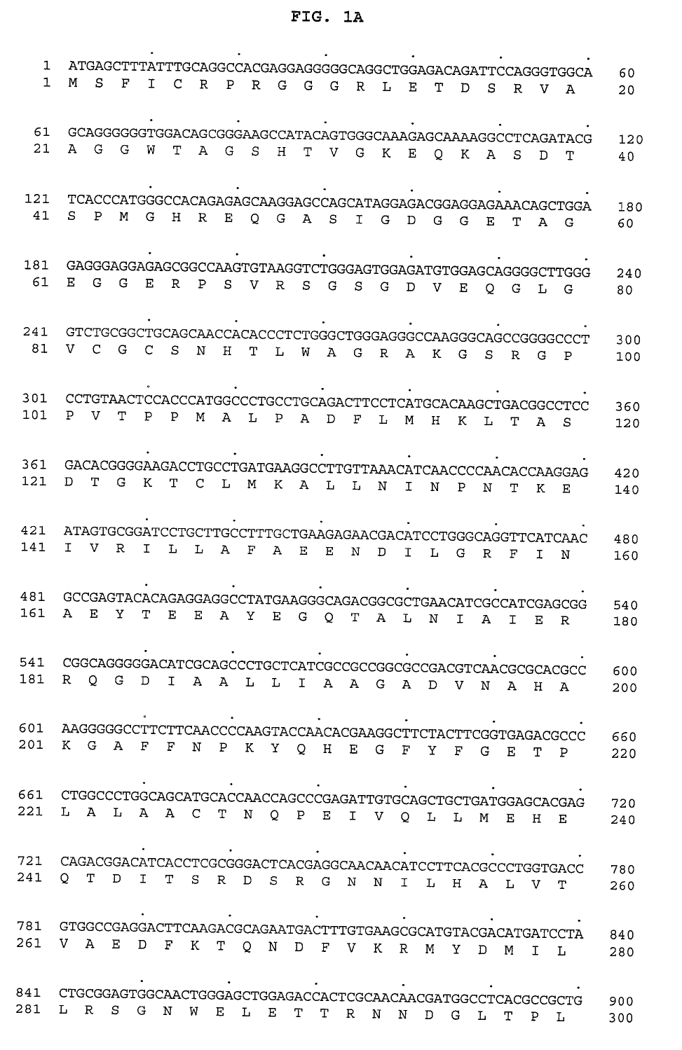

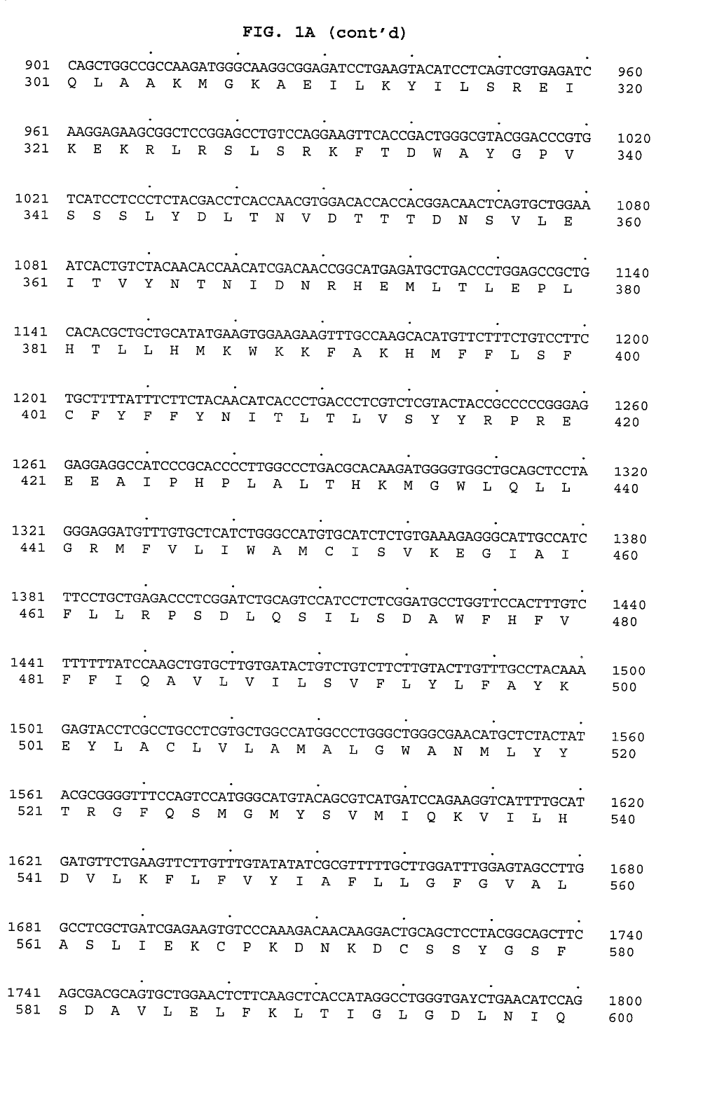

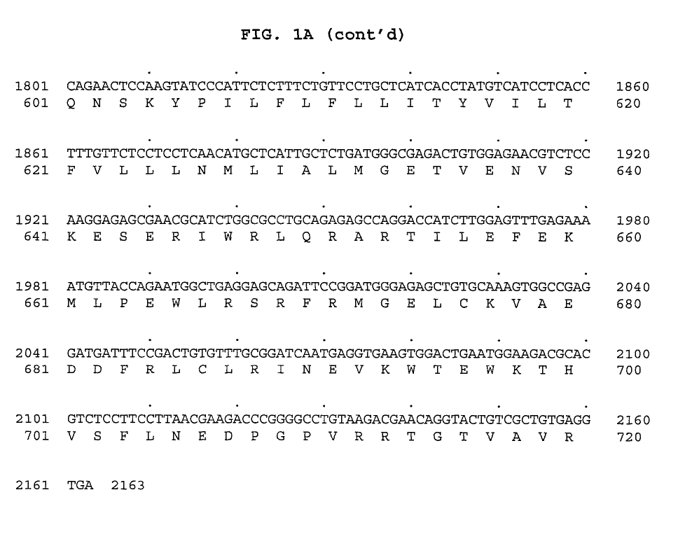

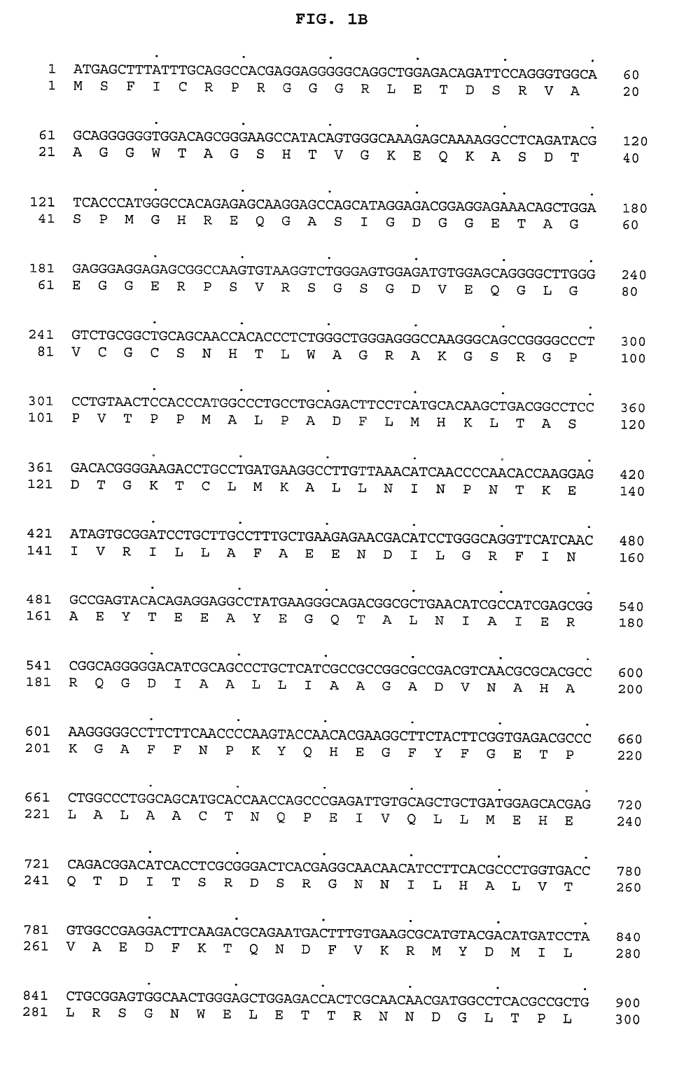

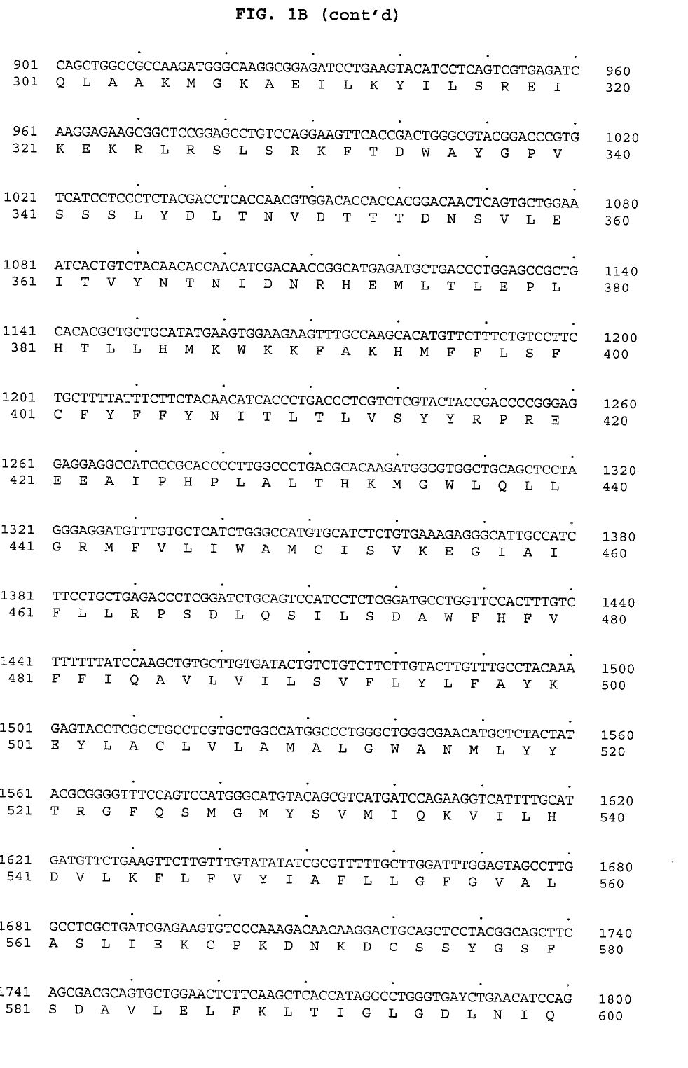

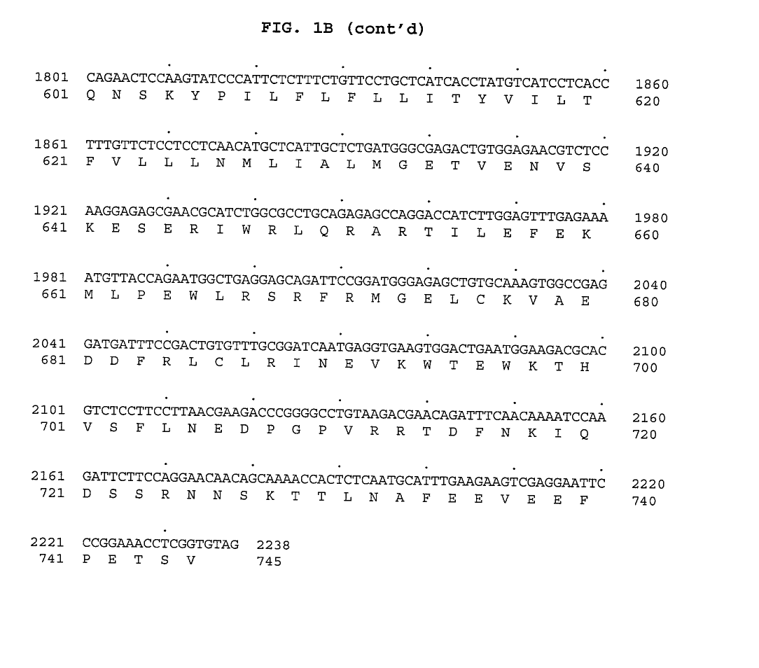

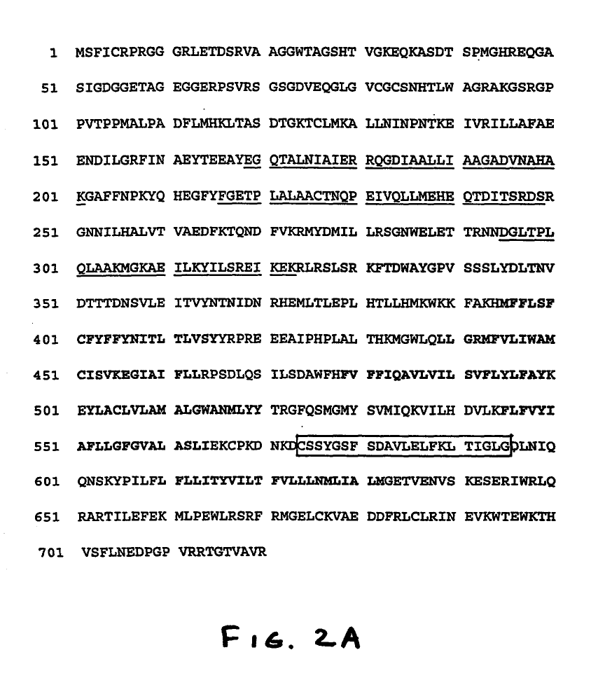

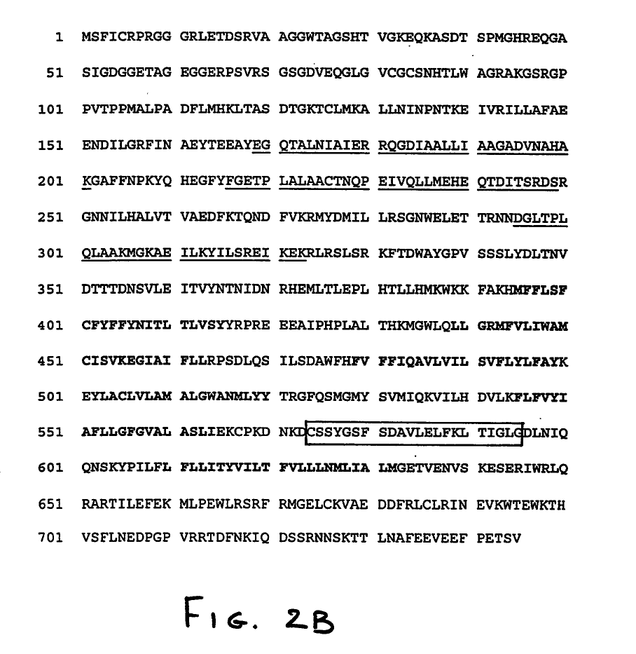

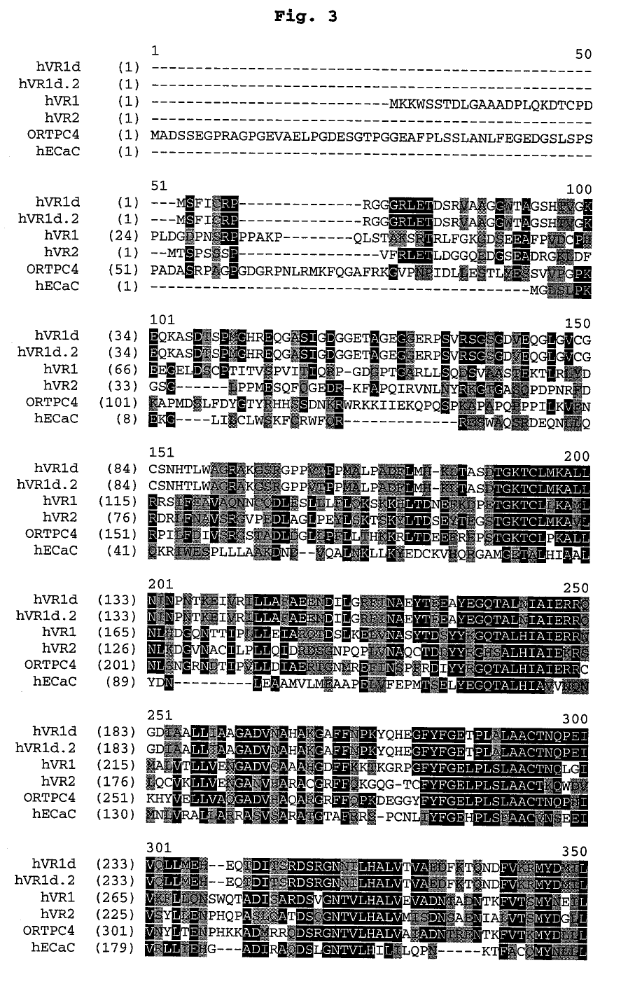

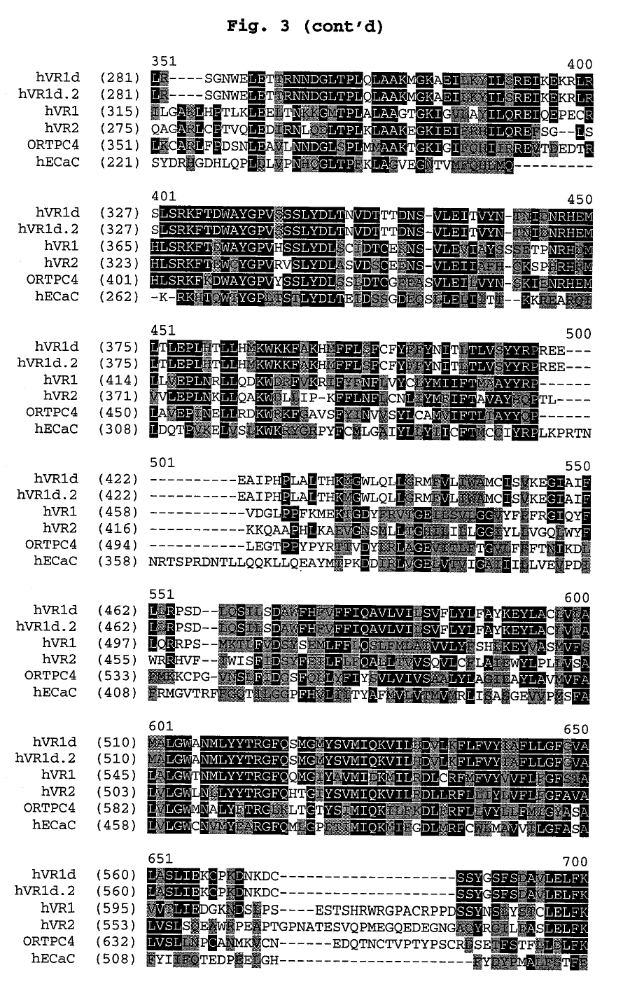

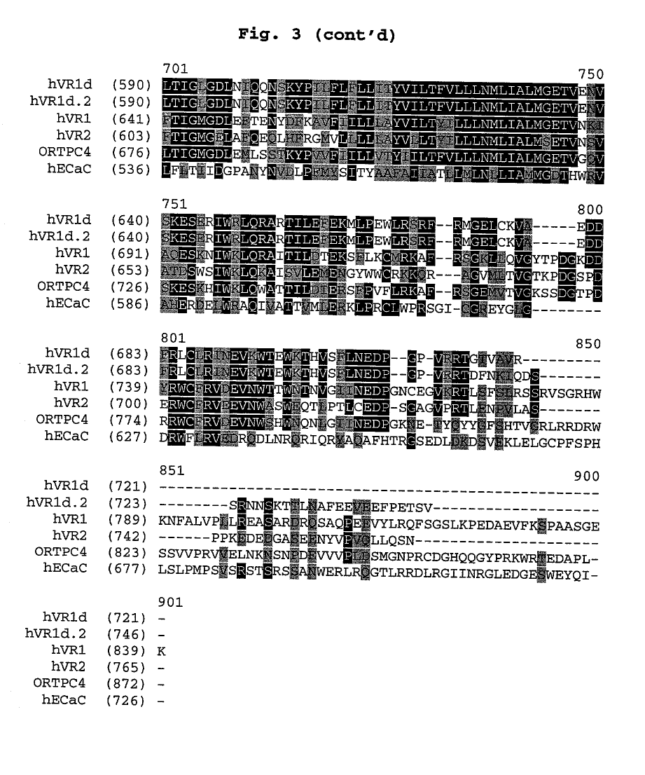

More specifically, disclosed herein are the DNA sequences of two splice variants of the hVR1d gene of the invention. These variants are referred to herein as hVR1d.1 and hVR1d.2 (see FIGS. 1A and 1B). The hVR1d.2 DNA sequence contains an additional 25 base pairs at the 3′ end of the molecule as compared to the hVR1d.1 DNA sequence. The corresponding hVR1d.1 and hVR1d.2 proteins are identical in amino acid sequence until amino acid residue 715, at which point hVRd1.1 contains a six amino acid C terminal sequence that differs from the 31 amino acid C terminal sequence of the hVR1d.2 protein (see FIGS. 2A and 2B).

The predicted molecular weight of the hVR1d.1 (FIG. 2A) polypeptide was determined to be about 81.3 kDa. The predicted molecular weight of the hVR1d.2 (FIG. 2B) polypeptide was determined to be about 84.3 kDa.

Polynucleotides corresponding to the encoding region of the hVR1d.1 are from nucleotide 1 to nucleotide 2160 of SEQ ID NO:1 (FIG. 2A). Polynucleotides corresponding to the encoding region of the hVR1d.2 are from nucleotide 1 to nucleotide 2235 of SEQ ID NO:1 (FIG. 2B).

In preferred embodiments, the present invention encompasses a polynucleotide lacking the initiating start codon, in addition to, the resulting encoded polypeptide of hVR1d.1. Specifically, the present invention encompasses the polynucleotide corresponding to nucleotides 4 thru 2160 of SEQ ID NO:1, and the polypeptide corresponding to amino acids 2 thru 720 of SEQ ID NO:2. Also encompassed are recombinant vectors comprising said encoding sequence, and host cells comprising said vector.

In preferred embodiments, the present invention encompasses a polynucleotide lacking the initiating start codon, in addition to, the resulting encoded polypeptide of hVR1d.2. Specifically, the present invention encompasses the polynucleotide corresponding to nucleotides 4 thru 2235 of SEQ ID NO:3, and the polypeptide corresponding to amino acids 2 thru 745 of SEQ ID NO:4. Also encompassed are recombinant vectors comprising said encoding sequence, and host cells comprising said vector.

The proteins corresponding to the hVR1d cDNAs of FIG. 1 show a modest level of homology to the human vanilloid receptor family of ion channels, e.g., an approximately 41-47% identity and 49-57% similarity to the reported VR1, VR2 and OTRPC4 proteins and an approximately 30-33% identity and 41-42% similarity to the reported EcaC and CaT1 and CaT2 proteins.

The hVR1d DNA sequences and encoded proteins of this invention also differ from the reported vanilloid family of ion channels in their patterns of tissue expression. For example, the hVR1d proteins of the invention are highly expressed in the spinal cord and brain tissues, such as the corpus callosum (CC), caudate nucleus (CN), and amygdala (A) of the brain (see FIG. 4).

The hVR1d proteins of the invention are predicted to contain six transmembrane domains as well as multiple consensus ankyrin domains (in the case of hVR1d, three ankyrin domains) in the N-terminal section of the protein, characteristic structural features of the TRP-vanilloid family of channels (see FIGS. 2A and 2B).

Specifically, the hVR1d.1 polypeptide was predicted to comprise six transmembrane domains (TM1 to TM6) located from about amino acid 395 to about amino acid 415 (TM1; SEQ ID NO:9); from about amino acid 439 to about amino acid 463 (TM2; SEQ ID NO:10); from about amino acid 479 to about amino acid 499 (TM3; SEQ ID NO:11); from about amino acid 502 to about amino acid 520 (TM4; SEQ ID NO:12); from about amino acid 545 to about amino acid 564 (TM5; SEQ ID NO:13); and/or from about amino acid 607 to about amino acid 625 (TM6; SEQ ID NO:14) of SEQ ID NO:2 (FIG. 2A). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

In preferred embodiments, the following transmembrane domain polypeptides are encompassed by the present invention: MFFLSFCFYFFYNITLTLVSY (SEQ ID NO:9), LLGRMFVLIWAMCISVKEGIAIFLL (SEQ ID NO:10), FVFFIQAVLVILSVFLYLFAY (SEQ ID NO:11), YLACLVLAMALGWANMLYY (SEQ ID NO:12), FLFVYIAFLLGFGVALASLI (SEQ ID NO:13), and/or ILFLFLLITYVILTFVLLL (SEQ ID NO:14). Polynucleotides encoding these polypeptides are also provided. The present invention also encompasses the use of these hVR1d.1 transmembrane domain polypeptides as immunogenic and/or antigenic epitopes as described elsewhere herein.

The present invention also encompasses the polypeptide sequences that intervene between each of the predicted hVR1d.1 transmembrane domains. Since these regions are solvent accessible either hVR1d.1 or intracellularly, they are particularly useful for designing antibodies specific to each region. Such antibodies may be useful as antagonists or agonists of the hVR1d.1 full-length polypeptide and may modulate its activity.

In preferred embodiments, the following inter-transmembrane domain polypeptides are encompassed by the present invention:

| | |
|---|---|
| YRPREEEAIPHPLALTHKMGWLQ | (SEQ ID NO:15), |
| RPSDLQSILSDAWFH | (SEQ ID NO:16), |
| TRGFQSMGMYSVMIQKVILHDVLKFLFVYIAFLLGFGVAL | (SEQ ID NO:17), and/or |
| EKCPKDNKDCSSYGSFSDAVLELFKLTIGLGDLNIQQNSKYP | (SEQ ID NO:18). |

Polynucleotides encoding these polypeptides are also provided. The present invention also encompasses the use of these hVR1d.1 intertransmembrane domain polypeptides as immunogenic and/or antigenic epitopes as described elsewhere herein.

Specifically, the hVR1d.2 polypeptide was also predicted to comprise six transmembrane domains (TM1 to TM6) located from about amino acid 395 to about amino acid 415 (TM1; SEQ ID NO:9); from about amino acid 439 to about amino acid 463 (TM2; SEQ ID NO:10); from about amino acid 479 to about amino acid 499 (TM3; SEQ ID NO:11); from about amino acid 502 to about amino acid 520 (TM4; SEQ ID NO:12); from about amino acid 545 to about amino acid 564 (TM5; SEQ ID NO:13); and/or from about amino acid 607 to about amino acid 625 (TM6; SEQ ID NO:14) of SEQ ID NO:4 (FIG. 2B). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

The present invention encompasses the use of the polypeptide corresponding to the ankyrin domain and the pore loop region delineated in FIGS. 2A and 2B as immunogenic and/or antigenic epitopes as described elsewhere herein.

Using known methods of protein engineering and recombinant DNA technology, variants may be generated to improve or alter the characteristics of the polypeptides of the present invention. For instance, one or more amino acids can be deleted from the N-terminus or C-terminus of the protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)).

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the protein will likely be retained when less than the majority of the residues of the protein are removed from the N-terminus or C-terminus. Whether a particular polypeptide lacking N- or C-terminal residues of a protein retains such immunogenic activities can readily be determined by routine methods described herein and otherwise known in the art.

Alternatively, such N-terminus or C-terminus deletions of a polypeptide of the present invention may, in fact, result in a significant increase in one or more of the biological activities of the polypeptide(s). For example, biological activity of many polypeptides are governed by the presence of regulatory domains at either one or both terminii. Such regulatory domains effectively inhibit the biological activity of such polypeptides in lieu of an activation event (e.g., binding to a cognate ligand or receptor, phosphorylation, proteolytic processing, etc.). Thus, by eliminating the regulatory domain of a polypeptide, the polypeptide may effectively be rendered biologically active in the absence of an activation event.

In preferred embodiments, the following N-terminal hVR1d.1 deletion polypeptides are encompassed by the present invention: M1-R720, S2-R720, F3-R720, I4-R720, C5-R720, R6-R720, P7-R720, R8-R720, G9-R720, G10-R720, G11-R720, R12-R720, L13-R720, E14-R720, T15-R720, D16-R720, S17-R720, R18-R720, V19-R720, A20-R720, A21-R720, G22-R720, G23-R720, W24-R720, T25-R720, A26-R720, G27-R720, S28-R720, H29-R720, T30-R720, V31-R720, G32-R720, K33-R720, E34-R720, Q35-R720, K36-R720, A37-R720, S38-R720, D39-R720, T40-R720, S41-R720, P42-R720, M43-R720, G44-R720, H45-R720, R46-R720, E47-R720, Q48-R720, G49-R720, A50-R720, S51-R720, I52-R720, G53-R720, D54-R720, G55-R720, G56-R720, E57-R720, T58-R720, A59-R720, G60-R720, E61-R720, G62-R720, G63-R720, E64-R720, R65-R720, P66-R720, S67-R720, V68-R720, R69-R720, S70-R720, G71-R720, S72-R720, G73-R720, D74-R720, V75-R720, E76-R720, Q77-R720, G78-R720, L79-R720, G80-R720, V81-R720, C82-R720, G83-R720, C84-R720, S85-R720, N86-R720, H87-R720, T88-R720, L89-R720, W90-R720, A91-R720, G92-R720, R93-R720, A94-R720, K95-R720, G96-R720, S97-R720, R98-R720, G99-R720, P100-R720, P101-R720, V102-R720, T103-R720, P104-R720, P105-R720, M106-R720, A107-R720, L108-R720, P109-R720, A110-R720, D111-R720, F112-R720, L113-R720, M114-R720, H115-R720, K116-R720, L117-R720, T118-R720, A119-R720, S120-R720, D121-R720, T122-R720, G123-R720, K124-R720, T125-R720, C126-R720, L127-R720, M128-R720, K129-R720, A130-R720, L131-R720, L132-R720, N133-R720, I134-R720, N135-R720, P136-R720, N137-R720, T138-R720, K139-R720, E140-R720, I141-R720, V142-R720, R143-R720, I144-R720, L145-R720, L146-R720, A147-R720, F148-R720, A149-R720, E150-R720, E151-R720, N152-R720, D153-R720, I154-R720, L155-R720, G156-R720, R157-R720, F158-R720, I159-R720, N160-R720, A161-R720, E162-R720, Y163-R720, T164-R720, E165-R720, E166-R720, A167-R720, Y168-R720, E169-R720, G170-R720, Q171-R720, T172-R720, A173-R720, L174-R720, N175-R720, I176-R720, A177-R720, I178-R720, E179-R720, R180-R720, R181-R720, Q182-R720, G183-R720, D184-R720, I185-R720, A186-R720, A187-R720, L188-R720, L189-R720, I190-R720, A191-R720, A192-R720, G193-R720, A194-R720, D195-R720, V196-R720, N197-R720, A198-R720, H199-R720, A200-R720, K201-R720, G202-R720, A203-R720, F204-R720, F205-R720, N206-R720, P207-R720, K208-R720, Y209-R720, Q210-R720, H211-R720, E212-R720, G213-R720, F214-R720, Y215-R720, F216-R720, G217-R720, E218-R720, T219-R720, P220-R720, L221-R720, A222-R720, L223-R720, A224-R720, A225-R720, C226-R720, T227-R720, N228-R720, Q229-R720, P230-R720, E231-R720, I232-R720, V233-R720, Q234-R720, L235-R720, L236-R720, M237-R720, E238-R720, H239-R720, E240-R720, Q241-R720, T242-R720, D243-R720, I244-R720, T245-R720, S246-R720, R247-R720, D248-R720, S249-R720, R250-R720, G251-R720, N252-R720, N253-R720, I254-R720, L255-R720, H256-R720, A257-R720, L258-R720, V259-R720, T260-R720, V261-R720, A262-R720, E263-R720, D264-R720, F265-R720, K266-R720, T267-R720, Q268-R720, N269-R720, D270-R720, F271-R720, V272-R720, K273-R720, R274-R720, M275-R720, Y276-R720, D277-R720, M278-R720, I279-R720, L280-R720, L281-R720, R282-R720, S283-R720, G284-R720, N285-R720, W286-R720, E287-R720, L288-R720, E289-R720, T290-R720, T291-R720, R292-R720, N293-R720, N294-R720, D295-R720, G296-R720, L297-R720, T298-R720, P299-R720, L300-R720, Q301-R720, L302-R720, A303-R720, A304-R720, K305-R720, M306-R720, G307-R720, K308-R720, A309-R720, E310-R720, I311-R720, L312-R720, K313-R720, Y314-R720, I315-R720, L316-R720, S317-R720, R318-R720, E319-R720, I320-R720, K321-R720, E322-R720, K323-R720, R324-R720, L325-R720, R326-R720, S327-R720, L328-R720, S329-R720, R330-R720, K331-R720, F332-R720, T333-R720, D334-R720, W335-R720, A336-R720, Y337-R720, G338-R720, P339-R720, V340-R720, S341-R720, S342-R720, S343-R720, L344-R720, Y345-R720, D346-R720, L347-R720, T348-R720, N349-R720, V350-R720, D351-R720, T352-R720, T353-R720, T354-R720, D355-R720, N356-R720, S357-R720, V358-R720, L359-R720, E360-R720, I361-R720, T362-R720, V363-R720, Y364-R720, N365-R720, T366-R720, N367-R720, I368-R720, D369-R720, N370-R720, R371-R720, H372-R720, E373-R720, M374-R720, L375-R720, T376-R720, L377-R720, E378-R720, P379-R720, L380-R720, H381-R720, T382-R720, L383-R720, L384-R720, H385-R720, M386-R720, K387-R720, W388-R720, K389-R720, K390-R720, F391-R720, A392-R720, K393-R720, H394-R720, M395-R720, F396-R720, F397-R720, L398-R720, S399-R720, F400-R720, C401-R720, F402-R720, Y403-R720, F404-R720, F405-R720, Y406-R720, N407-R720, I408-R720, T409-R720, L410-R720, T411-R720, L412-R720, V413-R720, S414-R720, Y415-R720, Y416-R720, R417-R720, P418-R720, R419-R720, E420-R720, E421-R720, E422-R720, A423-R720, I424-R720, P425-R720, H426-R720, P427-R720, L428-R720, A429-R720, L430-R720, T431-R720, H432-R720, K433-R720, M434-R720, G435-R720, W436-R720, L437-R720, Q438-R720, L439-R720, L440-R720, G441-R720, R442-R720, M443-R720, F444-R720, V445-R720, L446-R720, I447-R720, W448-R720, A449-R720, M450-R720, C451-R720, I452-R720, S453-R720, V454-R720, K455-R720, E456-R720, G457-R720, I458-R720, A459-R720, I460-R720, F461-R720, L462-R720, L463-R720, R464-R720, P465-R720, S466-R720, D467-R720, L468-R720, Q469-R720, S470-R720, I471-R720, L472-R720, S473-R720, D474-R720, A475-R720, W476-R720, F477-R720, H478-R720, F479-R720, V480-R720, F481-R720, F482-R720, I483-R720, Q484-R720, A485-R720, V486-R720, L487-R720, V488-R720, I489-R720, L490-R720, S491-R720, V492-R720, F493-R720, L494-R720, Y495-R720, L496-R720, F497-R720, A498-R720, Y499-R720, K500-R720, E501-R720, Y502-R720, L503-R720, A504-R720, C505-R720, L506-R720, V507-R720, L508-R720, A509-R720, M510-R720, A511-R720, L512-R720, G513-R720, W514-R720, A515-R720, N516-R720, M517-R720, L518-R720, Y519-R720, Y520-R720, T521-R720, R522-R720, G523-R720, F524-R720, Q525-R720, S526-R720, M527-R720, G528-R720, M529-R720, Y530-R720, S531-R720, V532-R720, M533-R720, I534-R720, Q535-R720, K536-R720, V537-R720, I538-R720, L539-R720, H540-R720, D541-R720, V542-R720, L543-R720, K544-R720, F545-R720, L546-R720, F547-R720, V548-R720, Y549-R720, I550-R720, A551-R720, F552-R720, L553-R720, L554-R720, G555-R720, F556-R720, G557-R720, V558-R720, A559-R720, L560-R720, A561-R720, S562-R720, L563-R720, I564-R720, E565-R720, K566-R720, C567-R720, P568-R720, K569-R720, D570-R720, N571-R720, K572-R720, D573-R720, C574-R720, S575-R720, S576-R720, Y577-R720, G578-R720, S579-R720, F580-R720, S581-R720, D582-R720, A583-R720, V584-R720, L585-R720, E586-R720, L587-R720, F588-R720, K589-R720, L590-R720, T591-R720, I592-R720, G593-R720, L594-R720, G595-R720, D596-R720, L597-R720, N598-R720, I599-R720, Q600-R720, Q601-R720, N602-R720, S603-R720, K604-R720, Y605-R720, P606-R720, I607-R720, L608-R720, F609-R720, L610-R720, F611-R720, L612-R720, L613-R720, I614-R720, T615-R720, Y616-R720, V617-R720, I618-R720, L619-R720, T620-R720, F621-R720, V622-R720, L623-R720, L624-R720, L625-R720, N626-R720, M627-R720, L628-R720, I629-R720, A630-R720, L631-R720, M632-R720, G633-R720, E634-R720, T635-R720, V636-R720, E637-R720, N638-R720, V639-R720, S640-R720, K641-R720, E642-R720, S643-R720, E644-R720, R645-R720, I646-R720, W647-R720, R648-R720, L649-R720, Q650-R720, R651-R720, A652-R720, R653-R720, T654-R720, I655-R720, L656-R720, E657-R720, F658-R720, E659-R720, K660-R720, M661-R720, L662-R720, P663-R720, E664-R720, W665-R720, L666-R720, R667-R720, S668-R720, R669-R720, F670-R720, R671-R720, M672-R720, G673-R720, E674-R720, L675-R720, C676-R720, K677-R720, V678-R720, A679-R720, E680-R720, D681-R720, D682-R720, F683-R720, R684-R720, L685-R720, C686-R720, L687-R720, R688-R720, I689-R720, N690-R720, E691-R720, V692-R720, K693-R720, W694-R720, T695-R720, E696-R720, W697-R720, K698-R720, T699-R720, H700-R720, V701-R720, S702-R720, F703-R720, L704-R720, N705-R720, E706-R720, D707-R720, P708-R720, G709-R720, P710-R720, V711-R720, R712-R720, R713-R720, and/or T714-R720 of SEQ ID NO:2. Polynucleotide sequences encoding these polypeptides are also provided. The present invention also encompasses the use of these N-terminal hVR1d.1 deletion polypeptides as immunogenic and/or antigenic epitopes as described elsewhere herein.

In preferred embodiments, the following C-terminal hVR1d.1 deletion polypeptides are encompassed by the present invention: M1-R720, M1-V719, M1-A718, M1-V717, M1-T716, M1-G715, M1-T714, M1-R713, M1-R712, M1-V711, M1-P710, M1-G709, M1-P708, M1-D707, M1-E706, M1-N705, M1-L704, M1-F703, M1-S702, M1-V701, M1-H700, M1-T699, M1-K698, M1-W697, M1-E696, M1-T695, M1-W694, M1-K693, M1-V692, M1-E691, M1-N690, M1-I689, M1-R688, M1-L687, M1-C686, M1-L685, M1-R684, M1-F683, M1-D682, M1-D681, M1-E680, M1-A679, M1-V678, M1-K677, M1-C676, M1-L675, M1-E674, M1-G673, M1-M672, M1-R671, M1-F670, M1-R669, M1-S668, M1-R667, M1-L666, M1-W665, M1-E664, M1-P663, M1-L662, M1-M661, M1-K660, M1-E659, M1-F658, M1-E657, M1-L656, M1-I655, M1-T654, M1-R653, M1-A652, M1-R651, M1-Q650, M1-L649, M1-R648, M1-W647, M1-I646, M1-R645, M1-E644, M1-S643, M1-E642, M1-K641, M1-S640, M1-V639, M1-N638, M1-E637, M1-V636, M1-T635, M1-E634, M1-G633, M1-M632, M1-L631, M1-A630, M1-I629, M1-L628, M1-M627, M1-N626, M1-L625, M1-L624, M1-L623, M1-V622, M1-F621, M1-T620, M1-L619, M1-I618, M1-V617, M1-Y616, M1-T615, M1-I614, M1-L613, M1-L612, M1-F611, M1-L610, M1-F609, M1-L608, M1-I607, M1-P606, M1-Y605, M1-K604, M1-S603, M1-N602, M1-Q601, M1-Q600, M1-I599, M1-N598, M1-L597, M1-D596, M1-G595, M1-L594, M1-G593, M1-I592, M1-T591, M1-L590, M1-K589, M1-F588, M1-L587, M1-E586, M1-L585, M1-V584, M1-A583, M1-D582, M1-S581, M1-F580, M1-S579, M1-G578, M1-Y577, M1-S576, M1-S575, M1-C574, M1-D573, M1-K572, M1-N571, M1-D570, M1-K569, M1-P568, M1-C567, M1-K566, M1-E565, M1-I564, M1-L563, M1-S562, M1-A561, M1-L560, M1-A559, M1-V558, M1-G557, M1-F556, M1-G555, M1-L554, M1-L553, M1-F552, M1-A551, M1-I550, M1-Y549, M1-V548, M1-F547, M1-L546, M1-F545, M1-K544, M1-L543, M1-V542, M1-D541, M1-H540, M1-L539, M1-I538, M1-V537, M1-K536, M1-Q535, M1-I534, M1-M533, M1-V532, M1-S531, M1-Y530, M1-M529, M1-G528, M1-M527, M1-S526, M1-Q525, M1-F524, M1-G523, M1-R522, M1-T521, M1-Y520, M1-Y519, M1-L518, M1-M517, M1-N516, M1-A515, M1-W514, M1-G513, M1-L512, M1-A511, M1-M510, M1-A509, M1-L508, M1-V507, M1-L506, M1-C505, M1-A504, M1-L503, M1-Y502, M1-E501, M1-K500, M1-Y499, M1-A498, M1-F497, M1-L496, M1-Y495, M1-L494, M1-F493, M1-V492, M1-S491, M1-L490, M1-I489, M1-V488, M1-L487, M1-V486, M1-A485, M1-Q484, M1-I483, M1-F482, M1-F481, M1-V480, M1-F479, M1-H478, M1-F477, M1-W476, M1-A475, M1-D474, M1-S473, M1-L472, M1-I471, M1-S470, M1-Q469, M1-L468, M1-D467, M1-S466, M1-P465, M1-R464, M1-L463, M1-L462, M1-F461, M1-I460, M1-A459, M1-I458, M1-G457, M1-E456, M1-K455, M1-V454, M1-S453, M1-I452, M1-C451, M1-M450, M1-A449, M1-W448, M1-I447, M1-L446, M1-V445, M1-F444, M1-M443, M1-R442, M1-G441, M1-L440, M1-L439, M1-Q438, M1-L437, M1-W436, M1-G435, M1-M434, M1-K433, M1-H432, M1-T431, M1-L430, M1-A429, M1-L428, M1-P427, M1-H426, M1-P425, M1-I424, M1-A423, M1-E422, M1-E421, M1-E420, M1-R419, M1-P418, M1-R417, M1-Y416, M1-Y415, M1-S414, M1-V413, M1-L412, M1-T411, M1-L410, M1-T409, M1-I408, M1-N407, M1-Y406, M1-F405, M1-F404, M1-Y403, M1-F402, M1-C401, M1-F400, M1-S399, M1-L398, M1-F397, M1-F396, M1-M395, M1-H394, M1-K393, M1-A392, M1-F391, M1-K390, M1-K389, M1-W388, M1-K387, M1-M386, M1-H385, M1-L384, M1-L383, M1-T382, M1-H381, M1-L380, M1-P379, M1-E378, M1-L377, M1-T376, M1-L375, M1-M374, M1-E373, M1-H372, M1-R371, M1-N370, M1-D369, M1-I368, M1-N367, M1-T366, M1-N365, M1-Y364, M1-V363, M1-T362, M1-I361, M1-E360, M1-L359, M1-V358, M1-S357, M1-N356, M1-D355, M1-T354, M1-T353, M1-T352, M1-D351, M1-V350, M1-N349, M1-T348, M1-L347, M1-D346, M1-Y345, M1-L344, M1-S343, M1-S342, M1-S341, M1-V340, M1-P339, M1-G338, M1-Y337, M1-A336, M1-W335, M1-D334, M1-T333, M1-F332, M1-K331, M1-R330, M1-S329, M1-L328, M1-S327, M1-R326, M1-L325, M1-R324, M1-K323, M1-E322, M1-K321, M1-I320, M1-E319, M1-R318, M1-S317, M1-L316, M1-I315, M1-Y314, M1-K313, M1-L312, M1-I311, M1-E310, M1-A309, M1-K308, M1-G307, M1-M306, M1-K305, M1-A304, M1-A303, M1-L302, M1-Q301, M1-L300, M1-P299, M1-T298, M1-L297, M1-G296, M1-D295, M1-N294, M1-N293, M1-R292, M1-T291, M1-T290, M1-E289, M1-L288, M1-E287, M1-W286, M1-N285, M1-G284, M1-S283, M1-R282, M1-L281, M1-L280, M1-I279, M1-M278, M1-D277, M1-Y276, M1-M275, M1-R274, M1-K273, M1-V272, M1-F271, M1-D270, M1-N269, M1-Q268, M1-T267, M1-K266, M1-F265, M1-D264, M1-E263, M1-A262, M1-V261, M1-T260, M1-V259, M1-L258, M1-A257, M1-H256, M1-L255, M1-I254, M1-N253, M1-N252, M1-G251, M1-R250, M1-S249, M1-D248, M1-R247, M1-S246, M1-T245, M1-I244, M1-D243, M1-T242, M1-Q241, M1-E240, M1-H239, M1-E238, M1-M237, M1-L236, M1-L235, M1-Q234, M1-V233, M1-I232, M1-E231, M1-P230, M1-Q229, M1-N228, M1-T227, M1-C226, M1-A225, M1-A224, M1-L223, M1-A222, M1-L221, M1-P220, M1-T219, M1-E218, M1-G217, M1-F216, M1-Y215, M1-F214, M1-G213, M1-E212, M1-H211, M1-Q210, M1-Y209, M1-K208, M1-P207, M1-N206, M1-F205, M1-F204, M1-A203, M1-G202, M1-K201, M1-A200, M1-H199, M1-A198, M1-N197, M1-V196, M1-D195, M1-A194, M1-G193, M1-A192, M1-A191, M1-I190, M1-L189, M1-L188, M1-A187, M1-A186, M1-I185, M1-D184, M1-G183, M1-Q182, M1-R181, M1-R180, M1-E179, M1-I178, M1-A177, M1-I176, M1-N175, M1-L174, M1-A173, M1-T172, M1-Q171, M1-G170, M1-E169, M1-Y168, M1-A167, M1-E166, M1-E165, M1-T164, M1-Y163, M1-E162, M1-A161, M1-N160, M1-I159, M1-F158, M1-R157, M1-G156, M1-L155, M1-I154, M1-D153, M1-N152, M1-E151, M1-E150, M1-A149, M1-F148, M1-A147, M1-L146, M1-L145, M1-I144, M1-R143, M1-V142, M1-I141, M1-E140, M1-K139, M1-T138, M1-N137, M1-P136, M1-N135, M1-I134, M1-N133, M1-L132, M1-L131, M1-A130, M1-K129, M1-M128, M1-L127, M1-C126, M1-T125, M1-K124, M1-G123, M1-T122, M1-D121, M1-S120, M1-A119, M1-T118, M1-L117, M1-K116, M1-H115, M1-M114, M1-L113, M1-F112, M1-D111, M1-A110, M1-P109, M1-L108, M1-A107, M1-M106, M1-P105, M1-P104, M1-T103, M1-V102, M1-P101, M1-P100, M1-G99, M1-R98, M1-S97, M1-G96, M1-K95, M1-A94, M1-R93, M1-G92, M1-A91, M1-W90, M1-L89, M1-T88, M1-H87, M1-N86, M1-S85, M1-C84, M1-G83, M1-C82, M1-V81, M1-G80, M1-L79, M1-G78, M1-Q77, M1-E76, M1-V75, M1-D74, M1-G73, M1-S72, M1-G71, M1-S70, M1-R69, M1-V68, M1-S67, M1-P66, M1-R65, M1-E64, M1-G63, M1-G62, M1-E61, M1-G60, M1-A59, M1-T58, M1-E57, M1-G56, M1-G55, M1-D54, M1-G53, M1-I52, M1-S51, M1-A50, M1-G49, M1-Q48, M1-E47, M1-R46, M1-H45, M1-G44, M1-M43, M1-P42, M1-S41, M1-T40, M1-D39, M1-S38, M1-A37, M1-K36, M1-Q35, M1-E34, M1-K33, M1-G32, M1-V31, M1-T30, M1-H29, M1-S28, M1-G27, M1-A26, M1-T25, M1-W24, M1-G23, M1-G22, M1-A21, M1-A20, M1-V19, M1-R18, M1-S17, M1-D16, M1-T15, M1-E14, M1-L13, M1-R12, M1-G11, M1-G10, M1-G9, M1-R8, and/or M1-P7 of SEQ ID NO:2. Polynucleotide sequences encoding these polypeptides are also provided. The present invention also encompasses the use of these C-terminal hVR1d.1 deletion polypeptides as immunogenic and/or antigenic epitopes as described elsewhere herein.

In preferred embodiments, the following N-terminal hVR1d.2 deletion polypeptides are encompassed by the present invention: M1-V745, S2-V745, F3-V745, I4-V745, C5-V745, R6-V745, P7-V745, R8-V745, G9-V745, G10-V745, G11-V745, R12-V745, L13-V745, E14-V745, T15-V745, D16-V745, S17-V745, R18-V745, V19-V745, A20-V745, A21-V745, G22-V745, G23-V745, W24-V745, T25-V745, A26-V745, G27-V745, S28-V745, H29-V745, T30-V745, V31-V745, G32-V745, K33-V745, E34-V745, Q35-V745, K36-V745, A37-V745, S38-V745, D39-V745, T40-V745, S41-V745, P42-V745, M43-V745, G44-V745, H45-V745, R46-V745, E47-V745, Q48-V745, G49-V745, A50-V745, S51-V745, I52-V745, G53-V745, D54-V745, G55-V745, G56-V745, E57-V745, T58-V745, A59-V745, G60-V745, E61-V745, G62-V745, G63-V745, E64-V745, R65-V745, P66-V745, S67-V745, V68-V745, R69-V745, S70-V745, G71-V745, S72-V745, G73-V745, D74-V745, V75-V745, E76-V745, Q77-V745, G78-V745, L79-V745, G80-V745, V81-V745, C82-V745, G83-V745, C84-V745, S85-V745, N86-V745, H87-V745, T88-V745, L89-V745, W90-V745, A91-V745, G92-V745, R93-V745, A94-V745, K95-V745, G96-V745, S97-V745, R98-V745, G99-V745, P100-V745, P101-V745, V102-V745, T103-V745, P104-V745, P105-V745, M106-V745, A107-V745, L108-V745, P109-V745, A110-V745, D111-V745, F112-V745, L113-V745, M114-V745, H115-V745, K116-V745, L117-V745, T118-V745, A119-V745, S120-V745, D121-V745, T122-V745, G123-V745, K124-V745, T125-V745, C126-V745, L127-V745, M128-V745, K129-V745, A130-V745, L131-V745, L132-V745, N133-V745, I134-V745, N135-V745, P136-V745, N137-V745, T138-V745, K139-V745, E140-V745, I141-V745, V142-V745, R143-V745, I144-V745, L145-V745, L146-V745, A147-V745, F148-V745, A149-V745, E150-V745, E151-V745, N152-V745, D153-V745, I154-V745, L155-V745, G156-V745, R157-V745, F158-V745, I159-V745, N160-V745, A161-V745, E162-V745, Y163-V745, T164-V745, E165-V745, E166-V745, A167-V745, Y168-V745, E169-V745, G170-V745, Q171-V745, T172-V745, A173-V745, L174-V745, N175-V745, I176-V745, A177-V745, I178-V745, E179-V745, R180-V745, R181-V745, Q182-V745, G183-V745, D184-V745, I185-V745, A186-V745, A187-V745, L188-V745, L189-V745, I190-V745, A191-V745, A192-V745, G193-V745, A194-V745, D195-V745, V196-V745, N197-V745, A198-V745, H199-V745, A200-V745, K201-V745, G202-V745, A203-V745, F204-V745, F205-V745, N206-V745, P207-V745, K208-V745, Y209-V745, Q210-V745, H211-V745, E212-V745, G213-V745, F214-V745, Y215-V745, F216-V745, G217-V745, E218-V745, T219-V745, P220-V745, L221-V745, A222-V745, L223-V745, A224-V745, A225-V745, C226-V745, T227-V745, N228-V745, Q229-V745, P230-V745, E231-V745, I232-V745, V233-V745, Q234-V745, L235-V745, L236-V745, M237-V745, E238-V745, H239-V745, E240-V745, Q241-V745, T242-V745, D243-V745, I244-V745, T245-V745, S246-V745, R247-V745, D248-V745, S249-V745, R250-V745, G251-V745, N252-V745, N253-V745, I254-V745, L255-V745, H256-V745, A257-V745, L258-V745, V259-V745, T260-V745, V261-V745, A262-V745, E263-V745, D264-V745, F265-V745, K266-V745, T267-V745, Q268-V745, N269-V745, D270-V745, F271-V745, V272-V745, K273-V745, R274-V745, M275-V745, Y276-V745, D277-V745, M278-V745, I279-V745, L280-V745, L281-V745, R282-V745, S283-V745, G284-V745, N285-V745, W286-V745, E287-V745, L288-V745, E289-V745, T290-V745, T291-V745, R292-V745, N293-V745, N294-V745, D295-V745, G296-V745, L297-V745, T298-V745, P299-V745, L300-V745, Q301-V745, L302-V745, A303-V745, A304-V745, K305-V745, M306-V745, G307-V745, K308-V745, A309-V745, E310-V745, I311-V745, L312-V745, K313-V745, Y314-V745, I315-V745, L316-V745, S317-V745, R318-V745, E319-V745, I320-V745, K321-V745, E322-V745, K323-V745, R324-V745, L325-V745, R326-V745, S327-V745, L328-V745, S329-V745, R330-V745, K331-V745, F332-V745, T333-V745, D334-V745, W335-V745, A336-V745, Y337-V745, G338-V745, P339-V745, V340-V745, S341-V745, S342-V745, S343-V745, L344-V745, Y345-V745, D346-V745, L347-V745, T348-V745, N349-V745, V350-V745, D351-V745, T352-V745, T353-V745, T354-V745, D355-V745, N356-V745, S357-V745, V358-V745, L359-V745, E360-V745, I361-V745, T362-V745, V363-V745, Y364-V745, N365-V745, T366-V745, N367-V745, I368-V745, D369-V745, N370-V745, R371-V745, H372-V745, E373-V745, M374-V745, L375-V745, T376-V745, L377-V745, E378-V745, P379-V745, L380-V745, H381-V745, T382-V745, L383-V745, L384-V745, H385-V745, M386-V745, K387-V745, W388-V745, K389-V745, K390-V745, F391-V745, A392-V745, K393-V745, H394-V745, M395-V745, F396-V745, F397-V745, L398-V745, S399-V745, F400-V745, C401-V745, F402-V745, Y403-V745, F404-V745, F405-V745, Y406-V745, N407-V745, I408-V745, T409-V745, L410-V745, T411-V745, L412-V745, V413-V745, S414-V745, Y415-V745, Y416-V745, R417-V745, P418-V745, R419-V745, E420-V745, E421-V745, E422-V745, A423-V745, I424-V745, P425-V745, H426-V745, P427-V745, L428-V745, A429-V745, L430-V745, T431-V745, H432-V745, K433-V745, M434-V745, G435-V745, W436-V745, L437-V745, Q438-V745, L439-V745, L440-V745, G441-V745, R442-V745, M443-V745, F444-V745, V445-V745, L446-V745, I447-V745, W448-V745, A449-V745, M450-V745, C451-V745, I452-V745, S453-V745, V454-V745, K455-V745, E456-V745, G457-V745, I458-V745, A459-V745, I460-V745, F461-V745, L462-V745, L463-V745, R464-V745, P465-V745, S466-V745, D467-V745, L468-V745, Q469-V745, S470-V745, I471-V745, L472-V745, S473-V745, D474-V745, A475-V745, W476-V745, F477-V745, H478-V745, F479-V745, V480-V745, F481-V745, F482-V745, I483-V745, Q484-V745, A485-V745, V486-V745, L487-V745, V488-V745, I489-V745, L490-V745, S491-V745, V492-V745, F493-V745, L494-V745, Y495-V745, L496-V745, F497-V745, A498-V745, Y499-V745, K500-V745, E501-V745, Y502-V745, L503-V745, A504-V745, C505-V745, L506-V745, V507-V745, L508-V745, A509-V745, M510-V745, A511-V745, L512-V745, G513-V745, W514-V745, A515-V745, N516-V745, M517-V745, L518-V745, Y519-V745, Y520-V745, T521-V745, R522-V745, G523-V745, F524-V745, Q525-V745, S526-V745, M527-V745, G528-V745, M529-V745, Y530-V745, S531-V745, V532-V745, M533-V745, I534-V745, Q535-V745, K536-V745, V537-V745, I538-V745, L539-V745, H540-V745, D541-V745, V542-V745, L543-V745, K544-V745, F545-V745, L546-V745, F547-V745, V548-V745, Y549-V745, I550-V745, A551-V745, F552-V745, L553-V745, L554-V745, G555-V745, F556-V745, G557-V745, V558-V745, A559-V745, L560-V745, A561-V745, S562-V745, L563-V745, I564-V745, E565-V745, K566-V745, C567-V745, P568-V745, K569-V745, D570-V745, N571-V745, K572-V745, D573-V745, C574-V745, S575-V745, S576-V745, Y577-V745, G578-V745, S579-V745, F580-V745, S581-V745, D582-V745, A583-V745, V584-V745, L585-V745, E586-V745, L587-V745, F588-V745, K589-V745, L590-V745, T591-V745, I592-V745, G593-V745, L594-V745, G595-V745, D596-V745, L597-V745, N598-V745, I599-V745, Q600-V745, Q601-V745, N602-V745, S603-V745, K604-V745, Y605-V745, P606-V745, I607-V745, L608-V745, F609-V745, L610-V745, F611-V745, L612-V745, L613-V745, I614-V745, T615-V745, Y616-V745, V617-V745, I618-V745, L619-V745, T620-V745, F621-V745, V622-V745, L623-V745, L624-V745, L625-V745, N626-V745, M627-V745, L628-V745, I629-V745, A630-V745, L631-V745, M632-V745, G633-V745, E634-V745, T635-V745, V636-V745, E637-V745, N638-V745, V639-V745, S640-V745, K641-V745, E642-V745, S643-V745, E644-V745, R645-V745, I646-V745, W647-V745, R648-V745, L649-V745, Q650-V745, R651-V745, A652-V745, R653-V745, T654-V745, I655-V745, L656-V745, E657-V745, F658-V745, E659-V745, K660-V745, M661-V745, L662-V745, P663-V745, E664-V745, W665-V745, L666-V745, R667-V745, S668-V745, R669-V745, F670-V745, R671-V745, M672-V745, G673-V745, E674-V745, L675-V745, C676-V745, K677-V745, V678-V745, A679-V745, E680-V745, D681-V745, D682-V745, F683-V745, R684-V745, L685-V745, C686-V745, L687-V745, R688-V745, I689-V745, N690-V745, E691-V745, V692-V745, K693-V745, W694-V745, T695-V745, E696-V745, W697-V745, K698-V745, T699-V745, H700-V745, V701-V745, S702-V745, F703-V745, L704-V745, N705-V745, E706-V745, D707-V745, P708-V745, G709-V745, P710-V745, V711-V745, R712-V745, R713-V745, T714-V745, D715-V745, F716-V745, N717-V745, K718-V745, I719-V745, Q720-V745, D721-V745, S722-V745, S723-V745, R724-V745, N725-V745, N726-V745, S727-V745, K728-V745, T729-V745, T730-V745, L731-V745, N732-V745, A733-V745, F734-V745, E735-V745, E736-V745, V737-V745, E738-V745, and/or E739-V745 of SEQ ID NO:4. Polynucleotide sequences encoding these polypeptides are also provided. The present invention also encompasses the use of these N-terminal hVR1d.2 deletion polypeptides as immunogenic and/or antigenic epitopes as described elsewhere herein.

In preferred embodiments, the following C-terminal hVR1d.2 deletion polypeptides are encompassed by the present invention: M1-V745, M1-S744, M1-T743, M1-E742, M1-P741, M1-F740, M1-E739, M1-E738, M1-V737, M1-E736, M1-E735, M1-F734, M1-A733, M1-N732, M1-L731, M1-T730, M1-T729, M1-K728, M1-S727, M1-N726, M1-N725, M1-R724, M1-S723, M1-S722, M1-D721, M1-Q720, M1-I719, M1-K718, M1-N717, M1-F716, M1-D715, M1-T714, M1-R713, M1-R712, M1-V711, M1-P710, M1-G709, M1-P708, M1-D707, M1-E706, M1-N705, M1-L704, M1-F703, M1-S702, M1-V701, M1-H700, M1-T699, M1-K698, M1-W697, M1-E696, M1-T695, M1-W694, M1-K693, M1-V692, M1-E691, M1-N690, M1-I689, M1-R688, M1-L687, M1-C686, M1-L685, M1-R684, M1-F683, M1-D682, M1-D681, M1-E680, M1-A679, M1-V678, M1-K677, M1-C676, M1-L675, M1-E674, M1-G673, M1-M672, M1-R671, M1-F670, M1-R669, M1-S668, M1-R667, M1-L666, M1-W665, M1-E664, M1-P663, M1-L662, M1-M661, M1-K660, M1-E659, M1-F658, M1-E657, M1-L656, M1-I655, M1-T654, M1-R653, M1-A652, M1-R651, M1-Q650, M1-L649, M1-R648, M1-W647, M1-I646, M1-R645, M1-E644, M1-S643, M1-E642, M1-K641, M1-S640, M1-V639, M1-N638, M1-E637, M1-V636, M1-T635, M1-E634, M1-G633, M1-M632, M1-L631, M1-A630, M1-I629, M1-L628, M1-M627, M1-N626, M1-L625, M1-L624, M1-L623, M1-V622, M1-F621, M1-T620, M1-L619, M1-I618, M1-V617, M1-Y616, M1-T615, M1-I614, M1-L613, M1-L612, M1-F611, M1-L610, M1-F609, M1-L608, M1-I607, M1-P606, M1-Y605, M1-K604, M1-S603, M1-N602, M1-Q601, M1-Q600, M1-I599, M1-N598, M1-L597, M1-D596, M1-G595, M1-L594, M1-G593, M1-I592, M1-T591, M1-L590, M1-K589, M1-F588, M1-L587, M1-E586, M1-L585, M1-V584, M1-A583, M1-D582, M1-S581, M1-F580, M1-S579, M1-G578, M1-Y577, M1-S576, M1-S575, M1-C574, M1-D573, M1-K572, M1-N571, M1-D570, M1-K569, M1-P568, M1-C567, M1-K566, M1-E565, M1-I564, M1-L563, M1-S562, M1-A561, M1-L560, M1-A559, M1-V558, M1-G557, M1-F556, M1-G555, M1-L554, M1-L553, M1-F552, M1-A551, M1-I550, M1-Y549, M1-V548, M1-F547, M1-L546, M1-F545, M1-K544, M1-L543, M1-V542, M1-D541, M1-H540, M1-L539, M1-I538, M1-V537, M1-K536, M1-Q535, M1-I534, M1-M533, M1-V532, M1-S531, M1-Y530, M1-M529, M1-G528, M1-M527, M1-S526, M1-Q525, M1-F524, M1-G523, M1-R522, M1-T521, M1-Y520, M1-Y519, M1-L518, M1-M517, M1-N516, M1-A515, M1-W514, M1-G513, M1-L512, M1-A511, M1-M510, M1-A509, M1-L508, M1-V507, M1-L506, M1-C505, M1-A504, M1-L503, M1-Y502, M1-E501, M1-K500, M1-Y499, M1-A498, M1-F497, M1-L496, M1-Y495, M1-L494, M1-F493, M1-V492, M1-S491, M1-L490, M1-I489, M1-V488, M1-L487, M1-V486, M1-A485, M1-Q484, M1-I483, M1-F482, M1-F481, M1-V480, M1-F479, M1-H478, M1-F477, M1-W476, M1-A475, M1-D474, M1-S473, M1-L472, M1-I471, M1-S470, M1-Q469, M1-L468, M1-D467, M1-S466, M1-P465, M1-R464, M1-L463, M1-L462, M1-F461, M1-I460, M1-A459, M1-I458, M1-G457, M1-E456, M1-K455, M1-V454, M1-S453, M1-I452, M1-C451, M1-M450, M1-A449, M1-W448, M1-I447, M1-L446, M1-V445, M1-F444, M1-M443, M1-R442, M1-G441, M1-L440, M1-L439, M1-Q438, M1-L437, M1-W436, M1-G435, M1-M434, M1-K433, M1-H432, M1-T431, M1-L430, M1-A429, M1-L428, M1-P427, M1-H426, M1-P425, M1-I424, M1-A423, M1-E422, M1-E421, M1-E420, M1-R419, M1-P418, M1-R417, M1-Y416, M1-Y415, M1-S414, M1-V413, M1-L412, M1-T411, M1-L410, M1-T409, M1-I408, M1-N407, M1-Y406, M1-F405, M1-F404, M1-Y403, M1-F402, M1-C401, M1-F400, M1-S399, M1-L398, M1-F397, M1-F396, M1-M395, M1-H394, M1-K393, M1-A392, M1-F391, M1-K390, M1-K389, M1-W388, M1-K387, M1-M386, M1-H385, M1-L384, M1-L383, M1-T382, M1-H381, M1-L380, M1-P379, M1-E378, M1-L377, M1-T376, M1-L375, M1-M374, M1-E373, M1-H372, M1-R371, M1-N370, M1-D369, M1-I368, M1-N367, M1-T366, M1-N365, M1-Y364, M1-V363, M1-T362, M1-I361, M1-E360, M1-L359, M1-V358, M1-S357, M1-N356, M1-D355, M1-T354, M1-T353, M1-T352, M1-D351, M1-V350, M1-N349, M1-T348, M1-L347, M1-D346, M1-Y345, M1-L344, M1-S343, M1-S342, M1-S341, M1-V340, M1-P339, M1-G338, M1-Y337, M1-A336, M1-W335, M1-D334, M1-T333, M1-F332, M1-K331, M1-R330, M1-S329, M1-L328, M1-S327, M1-R326, M1-L325, M1-R324, M1-K323, M1-E322, M1-K321, M1-I320, M1-E319, M1-R318, M1-S317, M1-L316, M1-I315, M1-Y314, M1-K313, M1-L312, M1-I311, M1-E310, M1-A309, M1-K308, M1-G307, M1-M306, M1-K305, M1-A304, M1-A303, M1-L302, M1-Q301, M1-L300, M1-P299, M1-T298, M1-L297, M1-G296, M1-D295, M1-N294, M1-N293, M1-R292, M1-T291, M1-T290, M1-E289, M1-L288, M1-E287, M1-W286, M1-N285, M1-G284, M1-S283, M1-R282, M1-L281, M1-L280, M1-I279, M1-M278, M1-D277, M1-Y276, M1-M275, M1-R274, M1-K273, M1-V272, M1-F271, M1-D270, M1-N269, M1-Q268, M1-T267, M1-K266, M1-F265, M1-D264, M1-E263, M1-A262, M1-V261, M1-T260, M1-V259, M1-L258, M1-A257, M1-H256, M1-L255, M1-I254, M1-N253, M1-N252, M1-G251, M1-R250, M1-S249, M1-D248, M1-R247, M1-S246, M1-T245, M1-I244, M1-D243, M1-T242, M1-Q241, M1-E240, M1-H239, M1-E238, M1-M237, M1-L236, M1-L235, M1-Q234, M1-V233, M1-I232, M1-E231, M1-P230, M1-Q229, M1-N228, M1-T227, M1-C226, M1-A225, M1-A224, M1-L223, M1-A222, M1-L221, M1-P220, M1-T219, M1-E218, M1-G217, M1-F216, M1-Y215, M1-F214, M1-G213, M1-E212, M 1-H211, M1-Q210, M1-Y209, M1-K208, M1-P207, M1-N206, M1-F205, M1-F204, M1-A203, M1-G202, M1-K201, M1-A200, M1-H199, M1-A198, M1-N197, M1-V196, M1-D195, M1-A194, M1-G193, M1-A192, M1-A191, M1-I190, M1-L189, M1-L188, M1-A187, M1-A186, M1-I185, M1-D184, M1-G183, M1-Q182, M1-R181, M1-R180, M1-E179, M1-I178, M1-A177, M1-I176, M1-N175, M1-L174, M1-A173, M1-T172, M1-Q171, M1-G170, M1-E169, M1-Y168, M1-A167, M1-E166, M1-E165, M1-T164, M1-Y163, M1-E162, M1-A161, M1-N160, M1-I159, M1-F158, M1-R157, M1-G156, M1-L155, M1-I154, M1-D153, M1-N152, M1-E151, M1-E150, M1-A149, M1-F148, M1-A147, M1-L146, M1-L145, M1-I144, M1-R143, M1-V142, M1-I141, M1-E140, M1-K139, M1-T138, M1-N137, M1-P136, M1-N135, M1-I134, M1-N133, M1-L132, M1-L131, M1-A130, M1-K129, M1-M128, M1-L127, M1-C126, M1-T125, M1-K124, M1-G123, M1-T122, M1-D121, M1-S120, M1-A119, M1-T118, M1-L117, M1-K116, M1-H115, M1-M114, M1-L113, M1-F112, M1-D111, M1-A110, M1-P109, M1-L108, M1-A107, M1-M106, M1-P105, M1-P104, M1-T103, M1-V102, M1-P101, M1-P100, M1-G99, M1-R98, M1-S97, M1-G96, M1-K95, M1-A94, M1-R93, M1-G92, M1-A91, M1-W90, M1-L89, M1-T88, M1-H87, M1-N86, M1-S85, M1-C84, M1-G83, M1-C82, M1-V81, M1-G80, M1-L79, M1-G78, M1-Q77, M1-E76, M1-V75, M1-D74, M1-G73, M1-S72, M1-G71, M1-S70, M1-R69, M1-V68, M1-S67, M1-P66, M1-R65, M1-E64, M1-G63, M1-G62, M1-E61, M1-G60, M1-A59, M1-T58, M1-E57, M1-G56, M1-G55, M1-D54, M1-G53, M1-I52, M1-S51, M1-A50, M1-G49, M1-Q48, M1-E47, M1-R46, M1-H45, M1-G44, M1-M43, M1-P42, M1-S41, M1-T40, M1-D39, M1-S38, M1-A37, M1-K36, M1-Q35, M1-E34, M1-K33, M1-G32, M1-V31, M1-T30, M1-H29, M1-S28, M1-G27, M1-A26, M1-T25, M1-W24, M1-G23, M1-G22, M1-A21, M1-A20, M1-V19, M1-R18, M1-S17, M1-D16, M1-T15, M1-E14, M1-L13, M1-R12, M1-G11, M1-G10, M1-G9, M1-R8, and/or M1-P7 of SEQ ID NO:4. Polynucleotide sequences encoding these polypeptides are also provided. The present invention also encompasses the use of these C-terminal hVR1d.2 deletion polypeptides as immunogenic and/or antigenic epitopes as described elsewhere herein.

In addition, the present invention provides the hVR1d clone corresponding to SEQ ID NO:1, deposited at the American Type Culture Collection (ATCC), 10801 University Boulevard, Manassas, Va. 20110-2209 on ______ and under ATCC Accession No. ______ according to the terms of the Budapest Treaty.

Other embodiments of the invention include antibodies directed to the hVR1d proteins and polypeptides of the invention, and methods and compositions for the diagnosis and treatment of human diseases related to ion channel dysfunction as described below.

### 5.1. The hVR1d Nucleic Acid Molecules of the Invention

The hVR1d nucleic acids of the invention, e.g., hVR1d.1 and hVR1d.2, are novel human nucleic acid molecules that encode proteins or polypeptides involved in the formation and/or function of novel human ion channels. Although these novel nucleic acids and proteins display some sequence and structural homology to the TRP and vanilloid families of cation channel proteins as well as other cation channel proteins known in the art, it is also known in the art that proteins displaying such homologies have significant differences in function, such as conductance and permeability, as well as differences in tissue expression. As such, it is acknowledged in the art that nucleic acid molecules and the proteins encoded by those molecules sharing these homologies can still represent diverse, distinct and unique nucleic acids and proteins, respectively.

The hVR1d nucleic acid molecules of the invention are those that comprise the following sequences: (a) the DNA sequence of hVR1d1.1 or hVR1d.2 as shown in FIGS. 1A or 1B, respectively; (b) any nucleic acid sequence that encodes the amino acid sequence of hVR1d.1 or hVR1d.2 as shown in FIGS. 2A or 2B, respectively; (c) any nucleic acid sequence that hybridizes to the complement of nucleic acid sequences that encode the amino acid sequences of FIGS. 2A or 2B under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (see, e.g., Ausubel F. M. et al., eds., 1989, Current Protocols in Molecular Biology, Vol. 1, Green Publishing Associates, Inc., and John Wiley & sons, Inc., New York, at p. 2.10.3) or (d) any nucleic acid sequence that hybridizes to the complement of nucleic acid sequences that encode the amino acid sequences of FIGS. 2A or 2B under less stringent conditions, such as moderately stringent conditions, e.g., washing in 0.2×SSC/0.1% SDS at 42° C. (Ausubel et al., 1989, supra), and which encodes a gene product functionally equivalent to a hVR1d gene product encoded by the sequences depicted in FIGS. 2A or 2B. “Functionally equivalent” as used herein refers to any protein capable of exhibiting a substantially similar in vivo or in vitro activity as the hVR1d gene products encoded by the hVR1d nucleic acid molecules described herein, e.g., ion channel formation or function.

As used herein, the term “hVR1d nucleic acid molecule” or “hVR1d nucleic acid” may also refer to fragments and/or degenerate variants of nucleic acid sequences (a) through (d), including naturally occurring variants or mutant alleles thereof. Such fragments include, for example, nucleic acid sequences that encode portions of the hVR1d protein that correspond to functional domains of the protein. One embodiment of such a hVR1d nucleic acid fragment comprises a nucleic acid containing a contiguous open reading frame, without introns, that encodes the fifth and sixth transmembrane segments of the hVR1d protein, including the predicted pore loop.

Additionally, the hVR1d nucleic acid molecules of the invention include isolated nucleic acids, preferably DNA molecules, that hybridize under highly stringent or moderately stringent hybridization conditions to at least about 6, preferably at least about 12, and more preferably at least about 18, consecutive nucleotides of the nucleic acid sequences of (a) through (d), identified supra.

The hVR1d nucleic acid molecules of the invention also include nucleic acids, preferably DNA molecules, that hybridize to, and are therefore complements of, the nucleic acid sequences of (a) through (d), supra. Such hybridization conditions may be highly stringent or moderately stringent, as described above. In those instances in which the nucleic acid molecules are deoxyoligonucleotides (“oligos”), highly stringent conditions may include, e.g., washing in 6×SSC/0.05% sodium pyrophosphate at 37° C. (for 14-base oligos), 48° C. (for 17-base oligos), 55° C. (for 20-base oligos), and 60° C. (for 23-base oligos). The nucleic acid molecules of the invention may encode or act as hVR1d antisense molecules useful, for example, in hVR1d gene regulation or as antisense primers in amplification reactions of hVR1d nucleic acid sequences. Further, such sequences may be used as part of ribozyme and/or triple helix sequences, also useful for hVR1d gene regulation. Still further, such molecules may be used as components of diagnostic methods whereby, for example, the presence of a particular hVR1d allele or alternatively-spliced hVR1d transcript responsible for causing or predisposing one to a disorder involving ion channel dysfunction may be detected.

Moreover, due to the degeneracy of the genetic code, other DNA sequences that encode substantially the amino acid sequences of hVR1d1.1 or hVR1d.2 may be used in the practice of the present invention for the cloning and expression of hVR1d polypeptides. Such DNA sequences include those that are capable of hybridizing to the hVR1d nucleic acids of this invention under stringent (high or moderate) conditions, or that would be capable of hybridizing under stringent conditions but for the degeneracy of the genetic code.

Typically, the hVR1d nucleic acids of the invention should exhibit at least about 80% overall sequence homology at the nucleotide level, more preferably at least about 85-90% overall homology and most preferably at least about 95% overall homology to the nucleic acid sequences of FIGS. 1A or 1B (as determined by the CLUSTAL W algorithm using default parameters (Thompson, J. D., et al., Nucleic Acids Research, 2(22):4673-4680, (1994)).

Altered hVR1d nucleic acid sequences that may be used in accordance with the invention include deletions, additions or substitutions of different nucleotide residues resulting in a modified nucleic acid molecule, i.e., mutated or truncated, that encodes the same or a functionally equivalent gene product as those described supra. The gene product itself may contain deletions, additions or substitutions of amino acid residues within the hVR1d protein sequence, which result in a silent change, thus producing a functionally equivalent hVR1d polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine, arginine and histidine; amino acids with uncharged polar head groups having similar hydrophilicity values include the following: leucine, isoleucine, valine, glycine, alanine, asparagine, glutamine, serine, threonine, phenylalanine, tyrosine. A functionally equivalent hVR1d polypeptide can include a polypeptide which displays the same type of biological activity (e.g., cation channel) as the native hVR1d protein, but not necessarily to the same extent.

The nucleic acid molecules or sequences of the invention may be engineered in order to alter the hVR1d coding sequence for a variety of ends including but not limited to alterations that modify processing and expression of the gene product. For example, mutations may be introduced using techniques which are well known in the art, e.g., site-directed mutagenesis, to insert new restriction sites, to alter glycosylation patterns, phosphorylation, etc. For example, in certain expression systems such as yeast, host cells may over-glycosylate the gene product. When using such expression systems, it may be preferable to alter the hVR1d coding sequence to eliminate any N-linked glycosylation sites.

In another embodiment, a hVR1d nucleic acid of the invention, e.g., a modified hVR1d nucleic acid, may be ligated to a heterologous protein-encoding sequence to encode a fusion protein. According to a preferred embodiment, a hVR1d nucleic acid of the invention that encodes a polypeptide with an activity of a hVR1d protein, or a fragment thereof, is linked, uninterrupted by stop codons and in frame, to a nucleotide sequence that encodes a heterologous protein or peptide. The fusion protein may be engineered to contain a cleavage site located between the hVR1d sequence and the heterologous protein sequence, so that the hVR1d protein can be cleaved away from the heterologous moiety. Nucleic acid sequences encoding fusion proteins of the invention may include full length hVR1d coding sequences, sequences encoding truncated hVR1d, sequences encoding mutated hVR1d or sequences encoding peptide fragments of hVR1d.

The hVR1d nucleic acid molecules of the invention can also be used as hybridization probes for obtaining hVR1d cDNAs or genomic hVR1d DNA. In addition, the nucleic acids of the invention can be used as primers in PCR amplification methods to isolate hVR1d cDNAs and genomic DNA, e.g., from other species.

The hVR1d gene sequences of the invention may also used to isolate mutant hVR1d gene alleles. Such mutant alleles may be isolated from individuals either known or proposed to have a genotype related to ion channel dysfunction. Mutant alleles and mutant allele gene products may then be utilized in the screening, therapeutic and diagnostic systems described in Section 5.4., infra. Additionally, such hVR1d gene sequences can be used to detect hVR1d gene regulatory (e.g., promoter) defects which can affect ion channel function.

A cDNA of a mutant hVR1d gene may be isolated, for example, by using PCR, a technique which is well known to those of skill in the art (see, e.g., U.S. Pat. No. 4,683,202). The first cDNA strand may be synthesized by hybridizing an oligo-dT oligonucleotide to mRNA isolated from tissue known or suspected to be expressed in an individual putatively carrying the mutant hVR1d allele, and by extending the new strand with reverse transcriptase. The second strand of the cDNA is then synthesized using an oligonucleotide that hybridizes specifically to the 5′ end of the normal gene. Using these two primers, the product is then amplified via PCR, cloned into a suitable vector, and subjected to DNA sequence analysis through methods well known in the art. By comparing the DNA sequence of the mutant hVR1d allele to that of the normal hVR1d allele, the mutation(s) responsible for the loss or alteration of function of the mutant hVR1d gene product can be ascertained.

Alternatively, a genomic library can be constructed using DNA obtained from an individual suspected of or known to carry the mutant hVR1d allele, or a cDNA library can be constructed using RNA from a tissue known, or suspected, to express the mutant hVR1d allele. The normal hVR1d gene or any suitable fragment thereof may then be labeled and used as a probe to identify the corresponding mutant hVR1d allele in such libraries. Clones containing the mutant hVR1d gene sequences may then be purified and subjected to sequence analysis according to methods well known in the art.

According to another embodiment, an expression library can be constructed utilizing cDNA synthesized from, for example, RNA isolated from a tissue known, or suspected, to express a mutant hVR1d allele in an individual suspected of or known to carry such a mutant allele. Gene products made by the putatively mutant tissue may be expressed and screened using standard antibody screening techniques in conjunction with antibodies raised against the normal hVR1d gene product, as described in Section 5.3, supra. For screening techniques, see, for example, Harlow, E. and Lane, eds., 1988, “Anti-bodies: A Laboratory Manual”, Cold Spring Harbor Press, Cold Spring Harbor.

In cases where a hVR1d mutation results in an expressed gene product with altered function (e.g., as a result of a missense or a frameshift mutation), a polyclonal set of anti-hVR1d gene product antibodies are likely to cross-react with the mutant hVR1d gene product. Library clones detected via their reaction with such labeled antibodies can be purified and subjected to sequence analysis according to methods well known to those of skill in the art.

In an alternate embodiment of the invention, the coding sequence of hVR1d can be synthesized in whole or in part, using chemical methods well known in the art, based on the nucleic acid and/or amino acid sequences of the hVR1d genes and proteins disclosed herein. See, for example, Caruthers et al., 1980, Nuc. Acids Res. Symp. Ser. 7: 215-233; Crea and Horn, 1980, Nuc. Acids Res. 9(10): 2331; Matteucci and Caruthers, 1980, Tetrahedron Letters 21: 719; and Chow and Kempe, 1981, Nuc. Acids Res. 9(12): 2807-2817. The invention also encompasses (a) DNA vectors that contain any of the foregoing hVR1d nucleic acids and/or their complements; (b) DNA expression vectors that contain any of the foregoing hVR1d coding sequences operatively associated with a regulatory element that directs the expression of the coding sequences; and (c) genetically engineered host cells that contain any of the foregoing hVR1d coding sequences operatively associated with a regulatory element that directs the expression of the coding sequences in the host cell. As used herein, regulatory elements include, but are not limited to inducible and non-inducible promoters, enhancers, operators and other elements known to those skilled in the art that drive and regulate expression. Such regulatory elements include but are not limited to the cytomegalovirus hCMV immediate early gene, the early or late promoters of SV40 adenovirus, the lac system, the T system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

The invention still further includes nucleic acid analogs, including but not limited to, peptide nucleic acid analogues, equivalent to the nucleic acid molecules described herein. “Equivalent” as used in this context refers to nucleic acid analogs that have the same primary base sequence as the nucleic acid molecules described above. Nucleic acid analogs and methods for the synthesis of nucleic acid analogs are well known to those of skill in the art. See, e.g., Egholm, M. et al., 1993, Nature 365:566-568; and Perry-O'Keefe, H. et al., 1996, Proc. Natl. Acad. USA 93:14670-14675.

### 5.2. hVR1d Proteins and Polypeptides

The hVR1d nucleic acid molecules of the invention may be used to generate recombinant DNA molecules that direct the expression in appropriate host cells of hVR1d polypeptides, including the full-length hVR1d proteins, e.g., hVR1d.1 or hVR1d.2, functionally active or equivalent hVR1d proteins and polypeptides, e.g., mutated, truncated or deleted forms of hVR1d, peptide fragments of hVR1d, or hVR1d fusion proteins. A functionally equivalent hVR1d polypeptide can include a polypeptide which displays the same type of biological activity (e.g., cation channel formation and/or function) as the native hVR1d protein, but not necessarily to the same extent.

In a preferred embodiment, the proteins and polypeptides of the invention include the hVR1d.1 and hVR1d.2 amino acid sequences depicted in FIGS. 2A and 2B, respectively. These sequences include six transmembrane domains and an overall topology that is conserved in the TRP-vanilloid family of ion channels. In addition, the amino acid sequences of FIGS. 2A and 2B contain three ankyrin domains in the N-terminal segment of the protein preceding the first transmembrane domain.

The hVR1 proteins and polypeptides of the invention include peptide fragments of hVR1d.1 or hVR1d.2, e.g., peptides corresponding to one or more domains of the protein, mutated, truncated or deleted forms of the proteins and polypeptides, as well as hVR1d fusion proteins, all of which derivatives of hVR1d can be obtained by techniques well known in the art, given the hVR1d nucleic acid and amino acid sequences disclosed herein. As noted in Section 5.1, supra, the proteins and polypeptides of the invention may contain deletions, additions or substitutions of amino acid residues within the hVR1d protein sequence, which result in a silent change, thus producing a functionally equivalent hVR1d polypeptide. Such amino acid substitutions may be made on the basis of similarity in polarity, charge, solubility, hydrophobicity, hydrophilicity, and/or the amphipatic nature of the residues involved. For example, negatively-charged amino acids include aspartic acid and glutamic acid; positively-charged amino acids include lysine, arginine and histidine; amino acids with uncharged polar head groups having similar hydrophilicity values include the following: leucine, isoleucine, valine, glycine, alanine, asparagine, glutamine, serine, threonine, phenylalanine, tyrosine.

Mutated or altered forms of the hVR1d proteins and polypeptides of the invention can be obtained using either random mutagenesis techniques or site-directed mutagenesis techniques well known in the art or by chemical methods, e.g., protein synthesis techniques (see Section 5.1, supra). Mutant hVR1d proteins or polypeptides can be engineered so that regions important for function are maintained, while variable residues are altered, e.g., by deletion or insertion of an amino acid residue(s) or by the substitution of one or more different amino acid residues. For example, conservative alterations at the variable positions of a polypeptide can be engineered to produce a mutant hVR1d polypeptide that retains the function of hVR1d. Non-conservative alterations of variable regions can be engineered to alter hVR1d function, if desired. Alternatively, in those cases where modification of function (either to increase or decrease function) is desired, deletion or non-conservative alterations of conserved regions of the polypeptide may be engineered.

Fusion proteins containing hVR1d amino acid sequences can also be obtained by techniques known in the art, including genetic engineering and chemical protein synthesis techniques. According to a preferred embodiment, the fusion proteins of the invention are encoded by an isolated nucleic acid molecule comprising an hVR1d nucleic acid of the invention that encodes a polypeptide with an activity of a hVR1d protein, or a fragment thereof, linked in frame and uninterrupted by stop codons to a nucleotide sequence that encodes a heterologous protein or peptide.

The fusion proteins of the invention include those that contain the full length hVR1d amino acid sequence, an hVR1d peptide sequence, e.g., encoding one or more functional domains, a mutant hVR1d amino acid sequence or a truncated hVR1d amino acid sequence linked to an unrelated protein or polypeptide sequence. Such fusion proteins include but are not limited to IgFc fusions which stabilize the hVR1d fusion protein and may prolong half-life of the protein in vivo or fusions to an enzyme, fluorescent protein or luminescent protein that provides a marker function.

According to a preferred embodiment of the invention, the hVR1d proteins and polypeptides, and derivatives thereof, of the invention are produced using genetic engineering techniques. Thus, in order to express a biologically active hVR1d polypeptide by recombinant technology, a nucleic acid molecule coding for the polypeptide, or a functional equivalent thereof as described in Section 5.1, supra, is inserted into an appropriate expression vector, i.e., a vector which contains the necessary elements for the transcription and translation of the inserted coding sequence. More specifically, the hVR1d nucleic acid is operatively associated with a regulatory nucleotide sequence containing transcriptional and/or translational regulatory information that controls expression of the hVR1d nucleic acid in the host cell. The hVR1d gene products so produced, as well as host cells or cell lines transfected or transformed with recombinant hVR1d expression vectors, can be used for a variety of purposes. These include but are not limited to generating antibodies (i.e., monoclonal or polyclonal) that bind to the hVR1d protein or polypeptide, including those that competitively inhibit binding and thus can “neutralize” hVR1d activity, and the screening and selection of hVR1d analogs or ligands.

Methods that are well known to those skilled in the art are used to construct expression vectors containing the hVR1d coding sequences of the invention and appropriate transcriptional and translational control elements and/or signals. These methods include in vitro recombinant DNA techniques, synthetic techniques and in vivo recombination/genetic recombination. See, for example, the techniques described in Maniatis et al., 1989, Molecular Cloning, A Laboratory Manual, Cold Spring Harbor Laboratory, N.Y. and Ausubel et al., 1989, Current Protocols in Molecular Biology, Greene Publishing Associates and Wiley Interscience, N.Y. See also Sambrook et al., 1989, Molecular Cloning, A Laboratory Manual, Cold Spring Harbor Press, N.Y.

A variety of host-expression vector systems may be used to express the hVR1d coding sequences of this invention. Such host-expression systems represent vehicles by which the coding sequences of interest may be produced and subsequently purified, but also represent cells which may, when transformed or transfected with the appropriate nucleotide coding sequences, exhibit the corresponding hVR1d gene products in situ and/or function in vivo. These hosts include but are not limited to microorganisms such as bacteria (e.g., E.coli, B. subtilis) transformed with recombinant bacteriophage DNA, plasmid DNA or cosmid DNA expression vectors containing the hVR1d coding sequences; yeast (e.g., Saccharomyces, Pichia) transformed with recombinant yeast expression vectors containing the hVR1d coding sequences; insect cell systems infected with recombinant virus expression vectors (e.g., baculovirus) containing the hVR1d coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the hVR1d coding sequences; or mammalian cell systems (e.g., COS, CHO, BHK, 293, 3T3) harboring recombinant expression constructs containing promoters derived from the genome of mammalian cells (e.g., the metallothionein promoter) or from mammalian viruses (e.g., the adenovirus late promoter or vaccinia virus 7.5K promoter).

The expression elements of these systems can vary in their strength and specificities. Depending on the host/vector system utilized, any of a number of suitable transcriptional and translational elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the hVR1d DNA, SV40-, BPV- and EBV-based vectors may be used with an appropriate selectable marker.

In bacterial systems, a number of expression vectors may be advantageously selected depending upon the use intended for the hVR1d polypeptide expressed. For example, when large quantities of an hVR1d polypeptide are to be produced, e.g., for the generation of antibodies or the production of the hVR1d gene product, vectors which direct the expression of high levels of fusion protein products that are readily purified may be desirable. Such vectors include but are not limited to the E. coli expression vector pUR278 (Ruther et al., 1983, EMBO J. 2: 1791), in which the hVR1d coding sequence may be ligated into the vector in frame with the lacZ coding region so that a hybrid hVR1d/lacZ protein is produced; pIN vectors (Inouye & Inouye, 1985, Nucleic Acids Res. 13: 3101-3109; Van Heeke & Schuster, 1989, J. Biol. Chem. 264: 5503-5509); and the like. pGEX vectors may also be used to express foreign polypeptides as fusion proteins with glutathione S-transferase (GST). In general, such fusion proteins are soluble and can easily be purified from lysed cells by affinity chromatography, e.g., adsorption to glutathione-agarose beads followed by elution in the presence of free glutathione. The pGEX vectors are designed to include thrombin or factor Xa protease cleavage sites so that the cloned polypeptide of interest can be released from the GST moiety. See also Booth et al., 1988, Immunol. Lett. 19: 65-70; and Gardella et al., 1990, J. Biol. Chem. 265: 15854-15859; Pritchett et al., 1989, Biotechniques 7: 580.

In yeast, a number of vectors containing constitutive or inducible promoters may be used. For a review, see Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Cold Spring Harbor Press, Vols. I and II.

In an insect system, Autographa californica nuclear polyhidrosis virus (AcNPV) can be used as a vector to express foreign genes. The virus grows in Spodoptera frugiperda cells. The hVR1d coding sequence may be cloned into non-essential regions (for example, the polyhedrin gene) of the virus and placed under control of an AcNPV promoter (for example, the polyhedrin promoter). Successful insertion of the hVR1d coding sequence will result in inactivation of the polyhedrin gene and production of non-occluded recombinant virus (i.e., virus lacking the proteinaceous coat coded for by the polyhedrin gene). These recombinant viruses can then be used to infect Spodoptera frugiperda cells in which the inserted gene is expressed (see e.g., Smith et al., 1983, J. Virol. 46: 584; Smith, U.S. Pat. No. 4,215,051).

In mammalian host cells, a number of viral-based expression systems may be utilized. In cases where an adenovirus is used as an expression vector, the hVR1d coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing hVR1d in infected hosts (see, e.g., Logan & Shenk, 1984, Proc. Natl. Acad. Sci. (USA) 81: 3655-3659). Alternatively, the vaccinia 7.5K promoter may be used (see, e.g., Mackett et al., 1982, Proc. Natl. Acad. Sci. (USA) 79: 7415-7419; Mackett et al., 1984, J. Virol. 49: 857-864; Panicali et al., 1982, Proc. Natl. Acad. Sci. 79: 4927- 4931).

Specific initiation signals may also be required for efficient translation of inserted hVR1d coding sequences. These signals include the ATG initiation codon and adjacent sequences. In cases where the entire hVR1d gene, including its own initiation codon and adjacent sequences, is inserted into the appropriate expression vector, no additional translational control signals may be needed. However, in cases where only a portion of the hVR1d coding sequence is inserted, exogenous translational control signals, including the ATG initiation codon, must be provided. Furthermore, the initiation codon must be in phase with the reading frame of the hVR1d coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, WI38, etc.

For long-term, high-yield production of recombinant proteins, stable expression is preferred. For example, cell lines which stably express the hVR1d polypeptides of this invention may be engineered. Thus, rather than using expression vectors which contain viral origins of replication, host cells can be transformed with hVR1d nucleic acid molecules, e.g., DNA, controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express hVR1d polypeptides on the cell surface. Such engineered cell lines are particularly useful in screening for hVR1d analogs or ligands.

In instances where the mammalian cell is a human cell, among the expression systems by which the hVR1d nucleic acid sequences of the invention can be expressed are human artificial chromosome (HAC) systems (see, e.g., Harrington et al., 1997, Nature Genetics 15: 345-355).

hVR1d gene products can also be expressed in transgenic animals such as mice, rats, rabbits, guinea pigs, pigs, micro-pigs, sheep, goats, and non-human primates, e.g., baboons, monkeys, and chimpanzees. The term “transgenic” as used herein refers to animals expressing hVR1d nucleic acid sequences from a different species (e.g., mice expressing human hVR1d nucleic acid sequences), as well as animals that have been genetically engineered to overexpress endogenous (i.e., same species) hVR1d nucleic acid sequences or animals that have been genetically engineered to no longer express endogenous hVR1d nucleic acid sequences (i.e., “knock-out” animals), and their progeny.

Transgenic animals according to this invention may be produced using techniques well known in the art, including but not limited to pronuclear microinjection (Hoppe, P. C. and Wagner, T. E., 1989, U.S. Pat. No. 4,873,191); retrovirus mediated gene transfer into germ lines (Van der Putten et al., 1985, Proc. Natl. Acad. Sci., USA 82: 6148-6152); gene targeting in embryonic stem cells (Thompson et al., 1989, Cell 56: 313-321); electroporation of embryos (Lo, 1983, Mol Cell. Biol. 3: 1803-1814); and sperm-mediated gene transfer (Lavitrano et al., 1989, Cell 57: 717-723); etc. For a review of such techniques, see Gordon, 1989, Transgenic Animals, Intl. Rev. Cytol. 115: 171-229.

In addition, any technique known in the art may be used to produce transgenic animal clones containing a hVR1d transgene, for example, nuclear transfer into enucleated oocytes of nuclei from cultured embryonic, fetal or adult cells induced to quiescence (Campbell et al., 1996, Nature 380: 64-66; Wilmut et al., 1997, Nature 385: 810-813).

Host cells which contain the hVR1d coding sequence and which express a biologically active gene product may be identified by at least four general approaches; (a) DNA-DNA or DNA-RNA hybridization; (b) the presence or absence of “marker” gene functions; (c) assessing the level of transcription as measured by the expression of hVR1d mRNA transcripts in the host cell; and (d) detection of the gene product as measured by immunoassay or by its biological activity.

In the first approach, the presence of the hVR1d coding sequence inserted in the expression vector can be detected by DNA-DNA or DNA-RNA hybridization using probes comprising nucleotide sequences that are homologous to the hVR1d coding sequence, respectively, or portions or derivatives thereof.

In the second approach, the recombinant expression vector/host system can be identified and selected based upon the presence or absence of certain “marker” gene functions. For example, if the hVR1d coding sequence is inserted within a marker gene sequence of the vector, recombinants containing the hVR1d coding sequence can be identified by the absence of the marker gene function. Alternatively, a marker gene can be placed in tandem with the hVR1d sequence under the control of the same or different promoter used to control the expression of the hVR1d coding sequence. Expression of the marker in response to induction or selection indicates expression of the hVR1d coding sequence.

Selectable markers include resistance to antibiotics, resistance to methotrexate, transformation phenotype, and occlusion body formation in baculovirus. In addition, thymidine kinase activity (Wigler et al., 1977, Cell 11: 223) hypoxanthine-guanine phosphoribosyltransferase (Szybalska & Szybalski, 1962, Proc. Natl. Acad. Sci. USA 48: 2026), and adenine phosphoribosyltransferase (Lowy et al., 1980, Cell 22: 817) genes can be employed in tk⁻, hgprt⁻ or aprt⁻ cells, respectively. Also, antimetabolite resistance can be used as the basis of selection for dhfr, which confers resistance to methotrexate (Wigler et al., 1980, Proc. Natl. Acad. Sci. USA 77: 3567; O'Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); neo, which confers resistance to the aminoglycoside G-418 (Colberre-Garapin, et al., 1981, J. Mol. Biol. 150: 1); and hygro, which confers resistance to hygromycin (Santerre et al., 1984, Gene 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85: 8047); and ODC (ornithine decarboxylase) which confers resistance to the ornithine decarboxylase inhibitor, 2-(difluoromethyl)-DL-ornithine, DFMO (McConlogue, 1987, in Current Communications in Molecular Biology, Cold Spring Harbor Laboratory ed.).

In the third approach, transcriptional activity for the hVR1d coding region can be assessed by hybridization assays. For example, RNA can be isolated and analyzed by Northern blot using a probe homologous to the hVR1d coding sequence or particular portions thereof. Alternatively, total nucleic acids of the host cell may be extracted and assayed for hybridization to such probes.

In the fourth approach, the expression of the hVR1d protein product can be assessed immunologically, for example by Western blots, immunoassays such as radioimmuno-precipitation, enzyme-linked immunoassays and the like. The ultimate test of the success of the expression system, however, involves the detection of biologically active hVR1d gene product. A number of assays can be used to detect hVR1d activity including but not limited to binding assays and biological assays for hVR1d activity.

Once a clone that produces high levels of a biologically active hVR1d polypeptide is identified, the clone may be expanded and used to produce large amounts of the polypeptide which may be purified using techniques well known in the art, including but not limited to, immunoaffinity purification using antibodies, immunoprecipitation or chromatographic methods including high performance liquid chromatography (HPLC).

Where the hVR1d coding sequence is engineered to encode a cleavable fusion protein, purification may be readily accomplished using affinity purification techniques. For example, a collagenase cleavage recognition consensus sequence may be engineered between the carboxy terminus of hVR1d and protein A. The resulting fusion protein may be readily purified using an IgG column that binds the protein A moiety. Unfused hVR1d may be readily released from the column by treatment with collagenase. Another example would be the use of pGEX vectors that express foreign polypeptides as fusion proteins with glutathionine S-transferase (GST). The fusion protein may be engineered with either thrombin or factor Xa cleavage sites between the cloned gene and the GST moiety. The fusion protein may be easily purified from cell extracts by adsorption to glutathione agarose beads followed by elution in the presence of glutathione. In fact, any cleavage site or enzyme cleavage substrate may be engineered between the hVR1d gene product sequence and a second peptide or protein that has a binding partner which could be used for purification, e.g., any antigen for which an immunoaffinity column can be prepared.

In addition, hVR1d fusion proteins may be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺ □nitriloacetic acid-agarose columns and histidine-tagged proteins are selectively eluted with imidazole-containing buffers.

Alternatively, the hVR1d proteins and polypeptides of the invention can be produced using chemical methods to synthesize the hVR1d amino acid sequences in whole or in part. For example, peptides can be synthesized by solid phase techniques, cleaved from the resin, and purified by preparative high performance liquid chromatography (see, e.g., Creighton, 1983, Proteins Structures And Molecular Principles, W. H. Freeman and Co., N.Y., pp. 50-60). The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49).

The hVR1d proteins, polypeptides and peptide fragments, mutated, truncated or deleted forms of hVR1d and/or hVR1d fusion proteins can be prepared for various uses, including but not limited to, the generation of antibodies, as reagents in diagnostic assays, the identification of other cellular gene products involved in ion transport, as reagents in assays for screening for compounds for use in the treatment of ion channel disorders.

### 5.3. Antibodies to hVR1d Polypeptides

The present invention also includes antibodies directed to the hVR1d polypeptides of this invention and methods for the production of those antibodies, including antibodies that specifically recognize one or more hVR1d epitopes or epitopes of conserved variants or peptide fragments of hVR1d.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a hVR1d protein or polypeptide in a biological sample and may, therefore, be utilized as part of a diagnostic or prognostic technique whereby patients may be tested for abnormal levels of hVR1d and/or for the presence of abnormal forms of the protein. Such antibodies may also be utilized in conjunction with, for example, compound screening protocols for the evaluation of the effect of test compounds on hVR1d levels and/or activity. Additionally, such antibodies can be used in conjunction with the gene therapy techniques described in Section 5.4, infra, to, for example, evaluate normal and/or genetically-engineered hVR1d-expressing cells prior to their introduction into the patient.

For the production of antibodies against hVR1d, various host animals may be immunized by injection with the protein or a portion thereof. Such host animals include rabbits, mice, rats, and baboons. Various adjuvants may be used to increase the immunological response, depending on the host species, including but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a hVR1d polypeptide, or an antigenic functional derivative thereof. For the production of polyclonal antibodies, host animals such as those described above, may be immunized by injection with the hVR1d polypeptide supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to, the hybridoma technique of Kohler and Milstein (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridomas producing the monoclonal antibodies of this invention may be cultivated in vitro or in vivo.

In addition, techniques developed for the production of chimeric antibodies (Morrison et al., 1984, Proc. Natl. Acad. Sci., 81: 6851-6855; Neuberger et al., 1984, Nature 312: 604-608; Takeda et al., 1985, Nature 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397.)

In addition, techniques have been developed for the production of humanized antibodies (see, e.g., Queen, U.S. Pat. No. 5,585,089). Humanized antibodies are antibody molecules from non-human species having one or more CDRs from the non-human species and a framework region from a human immunoglobulin molecule.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be used in the production of single chain antibodies against hVR1d. Single chain antibodies are formed by linking the heavy and light chain fragments of the Fv region via an amino acid bridge, resulting in a single chain polypeptide.

Furthermore, antibody fragments which recognize specific epitopes of hVR1d may be produced by techniques well known in the art. For example, such fragments include but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed (Huse et al., 1989, Science 246: 1275-1281) to allow rapid and easy identification of monoclonal Fab fragments with the desired specificity.

### 5.4. Uses of the hVR1d Nucleic Acid Molecules, Proteins and Polypeptides, and Antibodies of the Invention

As discussed supra, the hVR1d nucleic acid molecules of this invention encode proteins that are involved in the formation and/or function of ion channels, more particularly, cation channels. Given the importance of cations such as calcium, sodium or potassium in many cellular processes, the hVR1d nucleic acid molecules and proteins and polypeptides of this invention are useful for the diagnosis and treatment of a variety of human disease conditions which involve ion, more particularly, cation, channel dysfunction. For example, calcium plays a role in the release of neurotransmitters, hormones and other circulating factors, the expression of numerous regulatory genes as well as the cellular process of apoptosis or cell death. Potassium provides for neuroprotection and also affects insulin secretion. Sodium is involved in the regulation of normal neuronal action potential generation and propagation. Sodium channel blockers such as lidocaine are important analgesics. Therefore, cation channel dysfunction may play a role in many human diseases and disorders such as CNS disorders, e.g., degenerative neurological disorders such as Alzheimer's disease or Parkinson's disease, as well as other neurological disorders such chronic pain, anxiety and depression. Other diseases and disorders that can be affected by ion channel dysfunction include cardiac disorders, e.g., arrhythmia, diabetes, hypercalcemia, hypercalciuria, or ion channel dysfunction that is associated with immunological disoreders, GI tract disorders or renal or liver disease. As such, proteins that are involved in either the formation or function of these ion channels (and the nucleic acids that encode those proteins) are useful for the diagnosis and treatment of many human diseases.

Among the uses for the nucleic acid molecules, proteins and polypeptides of the invention are the prognostic and diagnostic evaluation of human disorders involving ion/cation channel dysfunction, and the identification of subjects with a predisposition to such disorders, as described below. Other uses include methods for the treatment of such ion/cation channel dysfunction disorders, for the modulation of hVR1d gene-mediated activity, and for the modulation of hVR1d-mediated effector functions.

In addition, the nucleic acid molecules and proteins and polypeptides of the invention can be used in assays for the identification of compounds which modulate the expression of the hVR1d genes of the invention and/or the activity of the hVR1d gene products. Such compounds can include, for example, other cellular products or small molecule compounds that are involved in cation homeostasis or activity.

### 5.4.1. Diagnosis and Prognosis of Ion-Related Disorders

Methods of the invention for the diagnosis and prognosis of human diseases involving ion, e.g., cation, dysfunction may utilize reagents such as the hVR1d nucleic acid molecules and sequences described in Sections 5.1, supra, or antibodies directed against hVR1d proteins or polypeptides, including peptide fragments thereof, as described in Section 5.3., supra. Specifically, such reagents may be used, for example, for: (1) the detection of the presence of hVR1d gene mutations, or the detection of either over- or under-expression of hVR1d gene mRNA relative to the non-cation dysfunctional state or the qualitative or quantitative detection of alternatively-spliced forms of hVR1d transcripts which may correlate with certain ion homeostasis disorders or susceptibility toward such disorders; and (2) the detection of either an over- or an under-abundance of hVR1d gene product relative to the non-cation dysfunctional state or the presence of a modified (e.g., less than full length) hVR1d gene product which correlates with a cation dysfunctional state or a progression toward such a state.

The methods described herein may be performed, for example, by utilizing pre-packaged diagnostic test kits comprising at least one specific hVR1d gene nucleic acid or anti-hVR1d gene antibody reagent described herein, which may be conveniently used, e.g., in clinical settings, to screen and diagnose patients exhibiting ion/cation channel/homeostasis abnormalities and to screen and identify those individuals exhibiting a predisposition to such abnormalities.

For the detection of hVR1d mutations, any nucleated cell can be used as a starting source for genomic nucleic acid. For the detection of hVR1d transcripts or hVR1d gene products, any cell type or tissue in which the hVR1d gene is expressed may be utilized.

Nucleic acid-based detection techniques are described in Section 5.4.1.1., infra, whereas peptide-based detection techniques are described in Section 5.4.1.2., infra.

### 5.4.1.1. Detection of hVR1d Gene Nucleic Acid Molecules

Mutations or polymorphisms within the hVR1d gene can be detected by utilizing a number of techniques. Nucleic acid from any nucleated cell can be used as the starting point for such assay techniques, and may be isolated according to standard nucleic acid preparation procedures which are well known to those of skill in the art.

Genomic DNA may be used in hybridization or amplification assays of biological samples to detect abnormalities involving hVR1d gene structure, including point mutations, insertions, deletions and chromosomal rearrangements. Such assays may include, but are not limited to, direct sequencing (Wong, C. et al., 1987, Nature 330:384-386), single stranded conformational polymorphism analyses (SSCP; Orita, M. et al., 1989, Proc. Natl. Acad. Sci. USA 86:2766-2770), heteroduplex analysis (Keen, T. J. et al., 1991, Genomics 11:199-205; Perry, D. J. & Carrell, R. W., 1992), denaturing gradient gel electrophoresis (DGGE; Myers, R. M. et al., 1985, Nucl. Acids Res. 13:3131-3145), chemical mismatch cleavage (Cotton, R. G. et al., 1988, Proc. Natl. Acad. Sci. USA 85:4397-4401) and oligonucleotide hybridization (Wallace, R. B. et al., 1981, Nucl. Acids Res. 9:879-894; Lipshutz, R. J. et al., 1995, Biotechniques 19:442-447).

Diagnostic methods for the detection of hVR1d gene-specific nucleic acid molecules, in patient samples or other appropriate cell sources, may involve the amplification of specific gene sequences, e.g., by PCR, followed by the analysis of the amplified molecules using techniques well known to those of skill in the art, such as, for example, those listed above. Utilizing analysis techniques such as these, the amplified sequences can be compared to those which would be expected if the nucleic acid being amplified contained only normal copies of the hVR1d gene in order to determine whether a hVR1d gene mutation exists.

Further, well-known genotyping techniques can be performed to type polymorphisms that are in close proximity to mutations in the hVR1d gene itself. These polymorphisms can be used to identify individuals in families likely to carry mutations. If a polymorphism exhibits linkage disequilibrium with mutations in the hVR1d gene, it can also be used to identify individuals in the general population likely to carry mutations. Polymorphisms that can be used in this way include restriction fragment length polymorphisms (RFLPs), which involve sequence variations in restriction enzyme target sequences, single-base polymorphisms and simple sequence repeat polymorphisms (SSLPs).

For example, Weber (U.S. Pat. No. 5,075,217) describes a DNA marker based on length polymorphisms in blocks of (dC-dA)n-(dG-dT)n short tandem repeats. The average separation of (dC-dA)n-(dG-dT)n blocks is estimated to be 30,000-60,000 bp. Markers which are so closely spaced exhibit a high frequency co-inheritance, and are extremely useful in the identification of genetic mutations, such as, for example, mutations within the hVR1d gene, and the diagnosis of diseases and disorders related to hVR1d mutations.

Also, Caskey et al. (U.S. Pat. No. 5,364,759) describe a DNA profiling assay for detecting short tri- and tetra-nucleotide repeat sequences. The process includes extracting the DNA of interest, such as the hVR1d gene, amplifying the extracted DNA, and labelling the repeat sequences to form a genotypic map of the individual's DNA.

A hVR1d probe could additionally be used to directly identify RFLPs. Additionally, a hVR1d probe or primers derived from the hVR1d sequences of the invention could be used to isolate genomic clones such as YACs, BACs, PACs, cosmids, phage or plasmids. The DNA contained in these clones can be screened for single-base polymorphisms or simple sequence length polymorphisms (SSLPs) using standard hybridization or sequencing procedures.

Alternative diagnostic methods for the detection of hVR1d gene-specific mutations or polymorphisms can include hybridization techniques which involve for example, contacting and incubating nucleic acids including recombinant DNA molecules, cloned genes or degenerate variants thereof, obtained from a sample, e.g., derived from a patient sample or other appropriate cellular source, with one or more labeled nucleic acid reagents including the hVR1d nucleic acid molecules of the invention including recombinant DNA molecules, cloned genes or degenerate variants thereof, as described in Section 5.1 supra, under conditions favorable for the specific annealing of these reagents to their complementary sequences within the hVR1d gene. Preferably, the lengths of these nucleic acid reagents are at least 15 to 30 nucleotides. After incubation, all non-annealed nucleic acids are removed from the nucleic acid:hVR1d molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. In this case, after incubation, non-annealed, labeled nucleic acid molecules of the invention as described in Section 5.1 are easily removed. Detection of the remaining, annealed, labeled hVR1d nucleic acid reagents is accomplished using standard techniques well-known to those in the art. The hVR1d gene sequences to which the nucleic acid molecules of the invention have annealed can be compared to the annealing pattern expected from a normal hVR1d gene sequence in order to determine whether a hVR1d gene mutation is present.

Quantitative and qualitative aspects of hVR1d gene expression can also be assayed. For example, RNA from a cell type or tissue known, or suspected, to express the hVR1d gene may be isolated and tested utilizing hybridization or PCR techniques s described supra. The isolated cells can be derived from cell culture or from a patient. The analysis of cells taken from culture may be a necessary step in the assessment of cells to be used as part of a cell-based gene therapy technique or, alternatively, to test the effect of compounds on the expression of the hVR1d gene. Such analyses may reveal both quantitative and qualitative aspects of the expression pattern of the hVR1d gene, including activation or inactivation of hVR1d gene expression and presence of alternatively spliced hVR1d transcripts.

In one embodiment of such a detection scheme, a cDNA molecule is synthesized from an RNA molecule of interest (e.g., by reverse transcription of the RNA molecule into cDNA). All or part of the resulting cDNA is then used as the template for a nucleic acid amplification reaction, such as a PCR amplification reaction, or the like. The nucleic acid reagents used as synthesis initiation reagents (e.g., primers) in the reverse transcription and nucleic acid amplification steps of this method are chosen from among the hVR1d nucleic acid molecules of the invention as described in Section 5.1, supra. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining protocol or e.g., quantitative PCR.

Such RT-PCR techniques can be utilized to detect differences in hVR1d transcript size which may be due to normal or abnormal alternative splicing. Additionally, such techniques can be utilized to detect quantitative differences between levels of full length and/or alternatively-spliced hVR1d transcripts detected in normal individuals relative to those individuals exhibiting ion dysfunction disorders or exhibiting a predisposition to toward such disorders.

In the case where detection of specific alternatively-spliced species is desired, appropriate primers and/or hybridization probes can be used, such that, in the absence of such sequence, no amplification would occur. Alternatively, primer pairs may be chosen utilizing the sequences depicted in FIGS. 1A or 1B to choose primers which will yield fragments of differing size depending on whether a particular exon is present or absent from the hVR1d transcript being utilized.

As an alternative to amplification techniques, standard Northern analyses can be performed if a sufficient quantity of the appropriate cells can be obtained. Utilizing such techniques, quantitative as well as size-related differences between hVR1d transcripts can also be detected.

Additionally, it is possible to perform hVR1d gene expression assays in situ, i.e., directly upon tissue sections (fixed and/or frozen) of patient tissue obtained from biopsies or resections, such that no nucleic acid purification is necessary. The nucleic acid molecules of the invention as described in Section 5.1 may be used as probes and/or primers for such in situ procedures (see, for example, Nuovo, G. J., 1992, “PCR In Situ Hybridization: Protocols And Applications”, Raven Press, NY).

### 5.4.1.2. Detection of hVR1d Gene Products

Antibodies directed against wild type or mutant hVR1d gene products or conserved variants or peptide fragments thereof as described supra may also be used for the diagnosis and prognosis of ion or cation-related disorders. Such diagnostic methods may be used to detect abnormalities in the level of hVR1d gene expression or abnormalities in the structure and/or temporal, tissue, cellular, or subcellular location of hVR1d gene products. Antibodies, or fragments of antibodies, may be used to screen potentially therapeutic compounds in vitro to determine their effects on hVR1d gene expression and hVR1d peptide production. The compounds which have beneficial effects on ion and cation-related disorders can be identified and a therapeutically effective dose determined.

In vitro immunoassays may be used, for example, to assess the efficacy of cell-based gene therapy for ion or cation-related disorders. For example, antibodies directed against hVR1d peptides may be used in vitro to determine the level of hVR1d gene expression achieved in cells genetically engineered to produce hVR1d peptides. Such analysis will allow for a determination of the number of transformed cells necessary to achieve therapeutic efficacy in vivo, as well as optimization of the gene replacement protocol.

The tissue or cell type to be analyzed will generally include those which are known, or suspected, to express the hVR1d gene. The protein isolation methods employed may, for example, be such as those described in Harlow, E. and Lane, D., 1988, “Antibodies: A Laboratory Manual”, Cold Spring Harbor Laboratory Press, Cold Spring Harbor, N.Y. The isolated cells can be derived from cell culture or from a patient. The analysis of cells taken from culture may be a necessary step in the assessment of cells to be used as part of a cell-based gene therapy technique or, alternatively, to test the effect of compounds on the expression of the hVR1d gene.

Preferred diagnostic methods for the detection of hVR1d gene products or conserved variants or peptide fragments thereof, may involve, for example, immunoassays wherein the hVR1d gene products or conserved variants, including gene products which are the result of alternatively-spliced transcripts, or peptide fragments are detected by their interaction with an anti-hVR1d gene product-specific antibody. For example, antibodies, or fragments of antibodies, such as those described in Section 5.3 supra, may be used to quantitatively or qualitatively detect the presence of hVR1d gene products or conserved variants or peptide fragments thereof. The antibodies (or fragments thereof) may, additionally, be employed histologically, as in immunofluorescence or immunoelectron microscopy, for in situ detection of hVR1d gene products or conserved variants or peptide fragments thereof. In situ detection may be accomplished by removing a histological specimen from a patient, and applying thereto a labeled hVR1d antibody of the present invention. The antibody (or fragment) is preferably applied by overlaying the labeled antibody (or fragment) onto a biological sample. Through the use of such a procedure, it is possible to determine not only the presence of the hVR1d gene product, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays for hVR1d gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of identifying hVR1d gene products or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled hVR1d gene specific antibody. The solid phase support may then be washed with the buffer a second time to remove unbound antibody. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-hVR1d gene product antibody may be determined according to well known methods. Those skilled in the art will be able to determine operative and optimal assay conditions for each determination by employing routine experimentation.

One of the ways in which the hVR1d gene peptide-specific antibody can be detectably labeled is by linking the antibody to an enzyme in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.,; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorometric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect hVR1d gene peptides through the use of a radioimmunoassay (RIA) (see, for example, Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

### 5.4.2. Screening Assays for Compounds that Modulate hVR1d Activity

Screening assays can be used to identify compounds that modulate hVR1d activity. These compounds can include, but are not limited to, peptides, small organic or inorganic molecules or macromolecules such as nucleic acid molecules or proteins, and may be utilized, e.g., in the control of ion and cation-related disorders, in the modulation of cellular processes such as the release of neurotransmitters or other cellular regulatory factors, cell activation or regulation, cell death and changes in cell membrane properties. These compounds may also be useful, e.g., in elaborating the biological functions of hVR1d gene products, i.e., hVR1 proteins and polypeptides, modulating those biological functions and for ameliorating symptoms of ion or cation-related disorders.

The compositions of the invention include pharmaceutical compositions comprising one or more of these compounds. Such pharmaceutical compositions can be formulated as discussed in Section 5.5, infra.

More specifically, these compounds can include compounds that bind to hVR1d gene products, compounds that bind to other proteins that interact with a hVR1d gene product and/or interfere with the interaction of the hVR1d gene product with other proteins, and compounds that modulate the activity of the hVR1d gene, i.e., modulate the level of hVR1d gene expression and/or modulate the level of hVR1d gene product or protein activity.

For example, assays may be utilized that identify compounds that bind to hVR1d gene regulatory sequences, e.g., promoter sequences (see e.g., Platt, K. A., 1994, J. Biol. Chem. 269:28558-28562), which compounds may modulate the level of hVR1d gene expression. In addition, functional assays can be used to screen for compounds that modulate hVR1d gene product activity. In such assays, compounds are screened for agonistic or antagonistic activity with respect to a biological activity or function of the hVR1d protein or polypeptide, such as changes in the intracellular levels of an ion or cation, changes in regulatory factor release, or other activities or functions of the hVR1d proteins and polypeptides of the invention.

According to a preferred embodiment, a Ca²⁺ flux assay can be utilized to monitor calcium uptake in hVR1d-expressing host cells. The host cells are pre-loaded with a Ca²⁺-sensitive fluorescently-labeled dye (e.g., Fluo-4, Fluo-3, Indo-1 or Fura-2), i.e., the intracellular calcium is fluorescently labelled with the dye, and the effect of the compound, e.g., on the intracellular levels of the labeled-calcium determined and compared to the intracellular levels of control cells, e.g., lacking exposure to the compound of interest. Compounds that have an agonistic, i.e., stimulatory, modulatory effect on hVR1d activity are those that, when contacted with the hVR1d-expressing cells, produce an increase in intracellular calcium relative to the control cells, whereas those compounds having an antagonistic modulatory effect on hVR1d activity will be those that produce a decrease in intracellular calcium.

Functional assays for monitoring the effects of compounds on the levels or flux of other ions can be similarly performed; for example, the levels of potassium can be monitored using rubidium influx.

Screening assays may also be designed to identify compounds capable of binding to the hVR1d gene product of the invention. Such compounds may be useful, e.g., in modulating the activity of wild type and/or mutant hVR1d gene products, in elaborating the biological function of the hVR1d gene product, and in screens for identifying compounds that disrupt normal hVR1d gene product interactions, or may in themselves disrupt such interactions.

The principle of such screening assays to identify compounds that bind to the hVR1d gene product involves preparing a reaction mixture of the hVR1d gene product and the test compound under conditions and for a time sufficient to allow the two components to interact with, i.e., bind to, and thus form a complex, which can represent a transient complex, which can be removed and/or detected in the reaction mixture. For example, one assay involves anchoring a hVR1d gene product or the test substance onto a solid phase and detecting hVR1d gene product/test compound complexes anchored on the solid phase at the end of the reaction. In one embodiment of such a method, the hVR1d gene product may be anchored onto a solid surface, and the test compound, which is not anchored, may be labeled, either directly or indirectly.

The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for hVR1d gene product or the test compound to anchor any complexes formed in solution, and a labeled antibody specific for the other component of the possible complex to detect anchored complexes.

Compounds that modulate hVR1d gene product activity can also include compounds that bind to proteins that interact with the hVR1d gene product. These modulatory compounds can be identified by first identifying those proteins that interact with the hVR1d gene product, e.g., by standard techniques known in the art for detecting protein-protein interactions, such as co-immunoprecipitation, crosslinking and co-purification through gradients or chromatographic columns. Utilizing procedures such as these allows for the isolation of proteins that interact with hVR1d gene products or polypeptides of the invention as described supra.

Once isolated, such a protein can be identified and can, in turn, be used, in conjunction with standard techniques, to identify additional proteins with which it interacts. For example, at least a portion of the amino acid sequence of the protein that interacts with the hVR1d gene product can be ascertained using techniques well known to those of skill in the art, such as via the Edman degradation technique (see, e.g., Creighton, 1983, “Proteins: Structures and Molecular Principles”, W. H. Freeman & Co., N.Y., pp.34-49). The amino acid sequence thus obtained may be used as a guide for the generation of oligonucleotide mixtures that can be used to screen for gene sequences encoding such proteins. Screening may be accomplished, for example, by standard hybridization or PCR techniques. Techniques for the generation of oligonucleotide mixtures and screening are well-known (see, e.g., Ausubel, supra., and PCR Protocols: A Guide to Methods and Applications, 1990, Innis, M. et al., eds. Academic Press, Inc., New York).

Additionally, methods may be employed that result in the simultaneous identification of genes which encode proteins interacting with hVR1d gene products or polypeptides. These methods include, for example, probing expression libraries with labeled hVR1d protein or polypeptide, using hVR1d protein or polypeptide in a manner similar to the well known technique of antibody probing of λgt11 libraries. One method that detects protein interactions in vivo is the two-hybrid system. A version of this system in described by Chien et al., 1991, Proc. Natl. Acad. Sci. USA, 88:9578-9582 and is commercially available from Clontech (Palo Alto, Calif.).

In addition, compounds that disrupt hVR1d interactions with its interacting or binding partners, as determined immediately above, may be useful in regulating the activity of the hVR1d gene product, including mutant hVR1d proteins and polypeptide. Such compounds may include, but are not limited to, molecules such as peptides, and the like, which may bind to the hVR1d gene product as described above.

The basic principle of the assay systems used to identify compounds that interfere with the interaction between the hVR1d gene product and its interacting partner or partners involves preparing a reaction mixture containing the hVR1d gene product, and the interacting partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of hVR1d gene product and its interacting partner. Control reaction mixtures are incubated without the test compound or with a placebo. The formation of any complexes between the hVR1d gene product and the interacting partner is then detected. The formation of a complex in the control reaction, but not in the reaction mixture containing the test compound, indicates that the compound interferes with the interaction of the hVR1d gene product and the interacting partner. Additionally, complex formation within reaction mixtures containing the test compound and a normal hVR1d gene product may also be compared to complex formation within reaction mixtures containing the test compound and a mutant hVR1d gene product. This comparison may be important in those cases wherein it is desirable to identify compounds that disrupt interactions of mutant but not normal hVR1d proteins.

The assay for compounds that interfere with the interaction of hVR1d gene products and interacting partners can be conducted in a heterogeneous or homogeneous format. Heterogeneous assays involve anchoring either the hVR1d gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the hVR1d gene products and the interacting partners, e.g., by competition, can be identified by conducting the reaction in the presence of the test substance; i.e., by adding the test substance to the reaction mixture prior to or simultaneously with the hVR1d gene product and interacting partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the hVR1d gene product or the interacting partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the hVR1d gene product or interacting partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds which inhibit complex formation or which disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the interacting components to anchor any complexes formed in solution, and a labeled antibody specific for the other partner to detect anchored complexes. Again, depending upon the order of addition of reactants to the liquid phase, test compounds that inhibit complex formation or that disrupt preformed complexes can be identified.

In an alternate embodiment, a preformed complex of the hVR1d gene protein and the interacting partner is prepared in which either the hVR1d gene product or its interacting partners is labeled, but the signal generated by the label is quenched due to complex formation (see, e.g., U.S. Pat. No. 4,109,496 by Rubenstein which utilizes this approach for immunoassays). The addition of a test substance that competes with and displaces one of the species from the preformed complex will result in the generation of a signal above background. In this way, test substances that disrupt hVR1d gene protein/interacting partner interaction can be identified.

In another embodiment of the invention, these same techniques can be employed using peptide fragments that correspond to the binding domains of the hVR1d protein and/or the interacting partner, in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interacting, e.g., binding. Alternatively, one protein can be anchored to a solid surface using methods described in this Section above, and allowed to interact with, e.g., bind, to its labeled interacting partner, which has been treated with a proteolytic enzyme, such as trypsin. After washing, a short, labeled peptide comprising the interacting, e.g., binding, domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

The human HVR1d polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a HVR1d polypeptide, or a bindable peptide fragment, of this invention, comprising providing a plurality of compounds, combining the HVR1d polypeptide, or a bindable peptide fragment, with each of a plurality of compounds for a time sufficient to allow binding under suitable conditions and detecting binding of the HVR1d polypeptide or peptide to each of the plurality of test compounds, thereby identifying the compounds that specifically bind to the HVR1d polypeptide or peptide.

Methods of identifying compounds that modulate the activity of the novel human HVR1d polypeptides and/or peptides are provided by the present invention and comprise combining a potential or candidate compound or drug modulator of calpain biological activity with an HVR1d polypeptide or peptide, for example, the HVR1d amino acid sequence as set forth in SEQ ID NOS:2, and measuring an effect of the candidate compound or drug modulator on the biological activity of the HVR1d polypeptide or peptide. Such measurable effects include, for example, physical binding interaction; the ability to cleave a suitable calpain substrate; effects on native and cloned HVR1d-expressing cell line; and effects of modulators or other calpain-mediated physiological measures.

Another method of identifying compounds that modulate the biological activity of the novel HVR1d polypeptides of the present invention comprises combining a potential or candidate compound or drug modulator of a calpain biological activity with a host cell that expresses the HVR1d polypeptide and measuring an effect of the candidate compound or drug modulator on the biological activity of the HVR1d polypeptide. The host cell can also be capable of being induced to express the HVR1d polypeptide, e.g., via inducible expression. Physiological effects of a given modulator candidate on the HVR1d polypeptide can also be measured. Thus, cellular assays for particular calpain modulators may be either direct measurement or quantification of the physical biological activity of the HVR1d polypeptide, or they may be measurement or quantification of a physiological effect. Such methods preferably employ a HVR1d polypeptide as described herein, or an overexpressed recombinant HVR1d polypeptide in suitable host cells containing an expression vector as described herein, wherein the HVR1d polypeptide is expressed, overexpressed, or undergoes upregulated expression.

Another aspect of the present invention embraces a method of screening for a compound that is capable of modulating the biological activity of a HVR1d polypeptide, comprising providing a host cell containing an expression vector harboring a nucleic acid sequence encoding a HVR1d polypeptide, or a functional peptide or portion thereof (e.g., SEQ ID NOS:2); determining the biological activity of the expressed HVR1d polypeptide in the absence of a modulator compound; contacting the cell with the modulator compound and determining the biological activity of the expressed HVR1d polypeptide in the presence of the modulator compound. In such a method, a difference between the activity of the HVR1d polypeptide in the presence of the modulator compound and in the absence of the modulator compound indicates a modulating effect of the compound.

Essentially any chemical compound can be employed as a potential modulator or ligand in the assays according to the present invention. Compounds tested as calpain modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel HVR1d polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptoids (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a HVR1d polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a HVR1d polypeptide or peptide to measure a biological binding or ligand binding activity, the source may be a whole cell lysate that can be prepared by successive freeze-thaw cycles (e.g., one to three) in the presence of standard protease inhibitors. The HVR1d polypeptide may be partially or completely purified by standard protein purification methods, e.g., affinity chromatography using specific antibody described infra, or by ligands specific for an epitope tag engineered into the recombinant HVR1d polypeptide molecule, also as described herein. Binding activity can then be measured as described.

Compounds which are identified according to the methods provided herein, and which modulate or regulate the biological activity or physiology of the HVR1d polypeptides according to the present invention are a preferred embodiment of this invention. It is contemplated that such modulatory compounds may be employed in treatment and therapeutic methods for treating a condition that is mediated by the novel HVR1d polypeptides by administering to an individual in need of such treatment a therapeutically effective amount of the compound identified by the methods described herein.

In addition, the present invention provides methods for treating an individual in need of such treatment for a disease, disorder, or condition that is mediated by the HVR1d polypeptides of the invention, comprising administering to the individual a therapeutically effective amount of the HVR1d-modulating compound identified by a method provided herein.

### 5.4.3. Methods and Compositions for the Treatment of Ion Channel-related Disorders

The present invention also relates to methods and compositions for the treatment or modulation of any disorder or cellular process that is mediated or regulated by hVR1d gene product expression or function, e.g., hVR1d-mediated cell activation, signal transduction, cellular regulatory factor release, etc. Further, hVR1d effector functions can be modulated via such methods and compositions.

The methods of the invention include methods that modulate hVR1d gene and gene product activity. In certain instances, the treatment will require an increase, upregulation or activation of hVR1d activity, while in other instances, the treatment will require a decrease, downregulation or suppression of hVR1d activity. “Increase” and “decrease” refer to the differential level of hVR1d activity relative to hVR1d activity in the cell type of interest in the absence of modulatory treatment. Methods for the decrease of hVR1d activity are discussed in Section 5.4.3.1, infra. Methods for the increase of hVR1d activity are discussed in Section 5.4.3.2, infra. Methods which can either increase or decrease hVR1d activity depending on the particular manner in which the method is practiced are discussed in Section 5.4.3.3, infra.

### 5.4.3.1 Methods for Decreasing hVR1d Activity

Successful treatment of ion channel/ionic homeostasis disorders, e.g., CNS disorders, cardiac disorders or hypercalcemia, can be brought about by methods which serve to decrease hVR1d activity. Activity can be decreased by, e.g., directly decreasing hVR1d gene product, i.e., protein, activity and/or by decreasing the level of hVR1d gene expression.

For example, compounds such as those identified through assays described in Section 5.4.2., supra, that decrease hVR1d gene product activity can be used in accordance with the invention to ameliorate symptoms associated with ion channel/ionic homeostasis disorders. As discussed supra, such molecules can include, but are not limited to peptides, including soluble peptides, and small organic or inorganic molecules, and can be referred to as hVR1d antagonists. Techniques for the determination of effective doses and administration of such compounds are described in Section 5.5., infra.

In addition, antisense and ribozyme molecules that inhibit hVR1d gene expression can also be used to reduce the level of hVR1d gene expression, thus effectively reducing the level of hVR1d gene product present, thereby decreasing the level of hVR1d protein activity. Still further, triple helix molecules can be utilized in reducing the level of hVR1d gene expression. Such molecules can be designed to reduce or inhibit either wild type, or if appropriate, mutant target gene activity. Techniques for the production and use of such molecules are well known to those of skill in the art.

Antisense approaches involve the design of oligonucleotides (either DNA or RNA) that are complementary to hVR1d gene mRNA. The antisense oligonucleotides will bind to the complementary hVR1d gene mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. See generally, Wagner, R., 1994, Nature 372:333-335. Thus, oligonucleotides complementary to either the 5′- or 3′-non-translated, non-coding regions of, e.g., the hVR1d.1 or hVR1d.2 nucleic acids depicted in FIG. 1 could be used in an antisense approach to inhibit translation of endogenous hVR1d gene mRNA.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of target or pathway gene mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and non-specific biological effects of oligonucleotides. It is also preferred that these studies compare levels of the target RNA or protein with that of an internal control RNA or protein. Additionally, results obtained using the antisense oligonucleotide are preferably compared with those obtained using a control oligonucleotide. It is preferred that the control oligonucleotide is of approximately the same length as the antisense oligonucleotide and that the nucleotide sequence of the control oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc.

The oligonucleotide may also include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Application No.

WO 88/09810) or the blood-brain barrier (see, e.g., PCT Application No. WO 89/10134), or hybridization-triggered cleavage agents (see, e.g., Krol et al., 1988, BioTechniques 6:958-976) or intercalating agents (see, e.g., Zon, 1988, Pharm. Res. 5:539-549). For example, the oligonucleotide may be conjugated to another molecule, e.g., a peptide, hybridization triggered cross-linking agent, transport agent, hybridization-triggered cleavage agent, etc.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209) and methylphosphonate oligonucleotides can be prepared by use of controlled pore glass polymer supports (Sarin et al., 1988, Proc. Natl. Acad. Sci. U.S.A. 85:7448-7451), etc.

The antisense molecules should be delivered to cells which express the hVR1d gene in vivo. A number of methods have been developed for delivering antisense DNA or RNA to cells; e.g., antisense molecules can be injected directly into the tissue site or modified antisense molecules designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Thus, a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous hVR1d gene transcripts and thereby prevent translation of the hVR1d gene mRNA. For example, a vector can be introduced in vivo such that it is taken up by a cell and directs the transcription of an antisense RNA.

Ribozymes are enzymatic RNA molecules capable of catalyzing the specific cleavage of RNA (For a review, see, e.g., Rossi, J., 1994, Current Biology 4:469-471). The mechanism of ribozyme action involves sequence-specific hybridization of the ribozyme molecule to complementary target RNA, followed by a endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyze endonucleolytic cleavage of RNA sequences encoding target gene proteins. Ribozyme molecules designed to catalytically cleave hVR1d gene mRNA transcripts can also be used to prevent translation of hVR1d gene mRNA and expression of target or pathway genes. (See, e.g., PCT Application No. WO 90/11364; Sarver et al., 1990, Science 247:1222- 1225).

The ribozymes of the present invention also include RNA endoribonucleases (hereinafter referred to as “Cech-type ribozymes”) such as the one which occurs naturally in Tetrahymena Thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; PCT Patent Application No. WO 88/04300; Been and Cech, 1986, Cell, 47:207-216). The Cech-type ribozymes have an eight base pair active site which hybridizes to a target RNA sequence, after which cleavage of the target RNA takes place. The invention encompasses those Cech-type ribozymes which target eight base-pair active site sequences that are present in an hVR1d gene.

As in the antisense approach, the ribozymes can be composed of modified oligonucleotides (e.g. for improved stability, targeting, etc.) and should be delivered to cells which express the hVR1d gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous hVR1d gene messages and inhibit translation. Because ribozymes, unlike antisense molecules, are catalytic, a lower intracellular concentration is required for efficiency.

Endogenous hVR1d gene expression can also be reduced by inactivating or “knocking out” the target and/or pathway gene or its promoter using targeted homologous recombination (see, e.g., Smithies et al., 1985, Nature 317:230-234; Thomas & Capecchi, 1987, Cell 51:503-512; Thompson et al., 1989 Cell 5:313-321). For example, a mutant, non-functional hVR1d gene (or a completely unrelated DNA sequence) flanked by DNA homologous to the endogenous hVR1d gene (either the coding regions or regulatory regions of the hVR1d gene) can be used, with or without a selectable marker and/or a negative selectable marker, to transfect cells that express the hVR1d gene in vivo. Insertion of the DNA construct, via targeted homologous recombination, results in inactivation of the hVR1d gene. Such techniques can also be utilized to generate ion/cation disorder animal models. It should be noted that this approach can be adapted for use in humans provided the recombinant DNA constructs are directly administered or targeted to the required site in vivo using appropriate viral vectors, e.g., herpes virus vectors.

Alternatively, endogenous hVR1d gene expression can be reduced by targeting deoxyribonucleotide sequences complementary to the regulatory region of the hVR1d gene (i.e., the hVR1d gene promoter and/or enhancers) to form triple helical structures that prevent transcription of the hVR1d gene in target cells in the body (see generally, Helene, C., 1991, Anticancer Drug Des. 6(6):569-84; Helene, C., et al., 1992, Ann. N.Y. Acad. Sci. 660:27-36; and Maher, L. J., 1992, Bioassays 14(12):807-15).

Nucleic acid molecules to be used in triple helix formation for the inhibition of transcription should be single stranded and composed of deoxynucleotides. The base composition of these oligonucleotides should be designed to promote triple helix formation via Hoogsteen base pairing rules, which generally require sizeable stretches of either purines or pyrimidines to be present on one strand of the duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands of the triplex.

Alternatively, the potential sequences that can be targeted for triple helix formation may be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of the duplex.

In instances wherein the antisense, ribozyme, and/or triple helix molecules described herein are utilized to inhibit mutant hVR1d gene expression, it is possible that the technique may so efficiently reduce or inhibit the transcription (triple helix) and/or translation (antisense, ribozyme) of mRNA produced by normal target gene alleles that the concentration of normal target gene product present may be lower than is necessary for a normal phenotype. In such cases, to ensure that substantially normal levels of hVR1d gene activity are maintained, nucleic acid molecules that encode and express hVR1d polypeptides exhibiting normal target gene activity can be introduced into cells via gene therapy methods that do not contain sequences susceptible to whatever antisense, ribozyme, or triple helix treatments are being utilized. In instances where the target gene encodes an extracellular protein, it can be preferable to coadminister normal target gene protein in order to maintain the requisite level of target gene activity.

Antisense RNA and DNA, ribozyme, and triple helix molecules of the invention can be prepared by any method known in the art, e.g., methods for chemically synthesizing oligodeoxyribonucleotides and oligoribonucleotides well known in the art such as solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

In addition, well-known modifications to DNA molecules can be introduced into the hVR1d nucleic acid molecules of the invention as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

### 5.4.3.2. Methods for Increasing hVR1d Activity

Successful treatment of ion/cation disorders can also be brought about by techniques which serve to increase the level of hVR1d activity. Activity can be increased by, for example, directly increasing hVR1d gene product activity and/or by increasing the level of hVR1d gene expression.

For example, compounds such as those identified through the assays described in Section 5.4.2., supra, that increase hVR1d activity can be used to treat ion/cation-related disorders. Such molecules can include, but are not limited to peptides, including soluble peptides, and small organic or inorganic molecules, and can be referred to as hVR1d agonists.

For example, a compound can, at a level sufficient to treat ion/cation-related disorders and symptoms, be administered to a patient exhibiting such symptoms. One of skill in the art will readily know how to determine the concentration of effective, non-toxic doses of the compound, utilizing techniques such as those described in Section 5.5, infra.

Alternatively, in instances wherein the compound to be administered is a peptide compound, DNA sequences encoding the peptide compound can be directly administered to a patient exhibiting an ion/cation-related disorder or symptoms, at a concentration sufficient to produce a level of peptide compound sufficient to ameliorate the symptoms of the disorder. Any of the techniques discussed infra, which achieve intracellular administration of compounds, such as, for example, liposome administration, can be utilized for the administration of such DNA molecules. In the case of peptide compounds which act extracellularly, the DNA molecules encoding such peptides can be taken up and expressed by any cell type, so long as a sufficient circulating concentration of peptide results for the elicitation of a reduction in the ion/cation disorder symptoms.

In cases where the ion/cation disorder can be localized to a particular portion or region of the body, the DNA molecules encoding such modulatory peptides may be administered as part of a delivery complex. Such a delivery complex can comprise an appropriate nucleic acid molecule and a targeting means. Such targeting means can comprise, for example, sterols lipids, viruses or target cell specific binding agents. Viral vectors can include, but are not limited to adenovirus, adeno-associated virus, and retrovirus vectors, in addition to other particles that introduce DNA into cells, such as liposomes.

Further, in instances wherein the ion/cation-related disorder involves an aberrant hVR1d gene, patients can be treated by gene replacement therapy. One or more copies of a normal hVR1d gene or a portion of the gene that directs the production of a normal hVR1d protein with normal hVR1d protein function, can be inserted into cells, via, for example a delivery complex as described supra.

Such gene replacement techniques can be accomplished either in vivo or in vitro. Techniques which select for expression within the cell type of interest are preferred. For in vivo applications, such techniques can, for example, include appropriate local administration of hVR1d gene sequences.

Additional methods which may be utilized to increase the overall level of hVR1d activity include the introduction of appropriate hVR1d gene-expressing cells, preferably autologous cells, into a patient at positions and in numbers which are sufficient to ameliorate the symptoms of the ion/cation-related disorder. Such cells may be either recombinant or non-recombinant. Among the cells which can be administered to increase the overall level of hVR1d gene expression in a patient are normal cells, which express the hVR1d gene. The cells can be administered at the anatomical site of expression, or as part of a tissue graft located at a different site in the body. Such cell-based gene therapy techniques are well known to those skilled in the art (see, e.g., Anderson, et al., U.S. Pat. No. 5,399,349; Mulligan and Wilson, U.S. Pat. No. 5,460,959).

hVR1d gene sequences can also be introduced into autologous cells in vitro. These cells expressing the hVR1d gene sequence can then be reintroduced, preferably by intravenous administration, into the patient until the disorder is treated and symptoms of the disorder are ameliorated.

### 5.4.3.3. Additional Modulatory Techniques

The present invention also includes modulatory techniques which, depending on the specific application for which they are utilized, can yield either an increase or a decrease in hVR1d activity levels leading to the amelioration of ion/cation-related disorders such as those described above.

Antibodies exhibiting modulatory capability can be utilized according to the methods of this invention to treat the ion/cation-related disorders. Depending on the specific antibody, the modulatory effect can be an increase or decrease in hVR1d activity. Such antibodies can be generated using standard techniques described in Section 5.3, supra, against full length wild type or mutant hVR1d proteins, or against peptides corresponding to portions of the proteins. The antibodies include but are not limited to polyclonal, monoclonal, Fab fragments, single chain antibodies, chimeric antibodies, etc.

Lipofectin or liposomes can be used to deliver the antibody or a fragment of the Fab region which binds to the hVR1d gene product epitope to cells expressing the gene product. Where fragments of the antibody are used, the smallest inhibitory fragment which binds to the hVR1d protein's binding domain is preferred. For example, peptides having an amino acid sequence corresponding to the domain of the variable region of the antibody that binds to the hVR1d protein can be used. Such peptides can be synthesized chemically or produced via recombinant DNA technology using methods well known in the art (e.g., see Creighton, 1983, supra and Sambrook et al., 1989, supra). Alternatively, single chain antibodies, such as neutralizing antibodies, which bind to intracellular epitopes can also be administered. Such single chain antibodies can be administered, for example, by expressing nucleotide sequences encoding single-chain antibodies within the target cell population by utilizing, for example, techniques such as those described in Marasco et al., 1993, Proc. Natl. Acad. Sci. USA 90:7889-7893.

### 5.5. Pharmaceutical Preparations and Methods of Administration

The compounds, e.g., nucleic acid sequences, proteins, polypeptides, peptides, and recombinant cells, described supra can be administered to a patient at therapeutically effective doses to treat or ameliorate ion/cation-related disorders. A therapeutically effective dose refers to that amount of a compound or cell population sufficient to result in amelioration of the disorder symptoms, or alternatively, to that amount of a nucleic acid sequence sufficient to express a concentration of hVR1d gene product which results in the amelioration of the disorder symptoms.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the methods of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. It is preferred that hVR1d-expressing cells be introduced into patients via intravenous administration.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

### 6. EXAMPLE

### Identification of a Novel hVR1d Gene and its Encoded Proteins

The section below describes the identification of novel human gene sequences encoding novel human ion channels.

### 6.1. Cloning of Novel hVR1d DNA Sequences

In general all routine molecular biology procedures followed standard protocols or relied on widely available commercial kits and reagents. All sequencing was done with an ABI 373 automated sequencer using commercial dye-terminator chemistry.

Known sequence data for hVR1a, hVR1b, hVR1c, and hVR2 were used to screen the EST and genomic public databases. The sequence search program used was gapped BLAST (S. F. Altschul et al., 1997, Nucleic Acids Res. 25: 3389-3402). The searches identified three Bacterial Artificial Chromsome (BAC) sequences in the public domain high throughput genomic database which contained segments having a significant similarity to but not identical with the query sequences. The accession numbers for these BACs are; AC025125, AC027040, and AC027796. The segments having similarity to the vanilloid family of receptors were searched against the nonredundant protein and nucleic acid databases and these segments were found to encode a potential novel vanilloid receptor. However, the sequence information obtained at this point was not sufficient to identify a complete coding sequence. Complete sequence data was then obtained using both 3′ and 5′ RACE procedures (M. A. Frohman et al., 1988, Proc. Natl. Acad. Sci. USA 85: 8998) and by sequencing cDNA clones isolated from a human brain library as follows:

A PCR primer pair designed from the genomic DNA sequences initially identified above as being homologous to vanilloid receptors, i.e., the BAC sequences, was used to screen a human brain cDNA library for potential cDNA clones. More specifically, a Frag3 primer pair, as follows, forward primer “Frag3-s” CGCAGTGCTGGAACTCTTCA (SEQ ID NO:19) and reverse primer “Frag3-a” CATCAGAGCAATGAGCATGTTGA (SEQ ID NO:20), in which the reverse primer contained biotin coupled to its 5′ end, was used to amplify a biotinylated fragment of hVR1d sequences from the genomic DNA. This DNA fragment was gel purified, denatured and then hybridized to a circular, single-stranded human brain cDNA library constructed using f1 helper phage following standard protocols. Hybridization was carried out at 42□C in 50% formamide, 1.5 M NaCl, 40 mM NaₓH_{y}PO₄ (pH 7.2), 5 mM EDTA, and 0.2% SDS.

Hybrids between the biotinylated DNA fragment and the circular DNA were captured on streptavidin magnetic beads. After thermal release from the beads, the single-stranded cDNA was converted to double-stranded form using a primer complementary to a T7 promoter sequence in the cDNA cloning vector. The double-stranded cDNA was then introduced into E. coli host cells by electroporation and the resulting colonies were screened by PCR, using the original primer pair, to identify the desired cDNA. Approximately 20 PCR positive clones were obtained. The insert size was determined for all of the clones and two clones with the largest inserts were selected for DNA sequencing.

Additional sequence information was obtained using the RACE method as cited above. More specifically, a human fetal brain Marathon□ cDNA library prepared by CLONTECH Laboratories, Inc. was used as a template. A nested PCR reaction was used to obtain 5′ sequence data. The two gene-specific primers, derived from the genomic sequence data, were “1D5R2” (GCCCAGGATGTCGTTCTCTTCAGC (SEQ ID NO:21)) in the first round of amplification and “1D5R3” (GATCCGCACTATCTCCTTGGTGTTGG (SEQ ID NO:22)) in the subsequent round. A single round of amplification was used to obtain 3′ sequence data using the gene-specific primer “1D3R2” (ACTGAATGGAAGACGCACGTCTCCTTC (SEQ ID NO:23)). For both the 5′ and 3′ RACE amplifications, CLONTECH'S primer “AP1” was used as the second primer. RACE products were cloned using Invitrogen Corporation's TOPO TA Cloning□ Kit following manufacturer's instructions. Insert size was assessed by restriction digest and clones having the largest inserts were then sequenced.

The nucleic acid sequences derived by these procedures are depicted in FIGS. 1A and 1B which identify, respectively, two splice variants of the coding sequence for novel cDNA clone hVR1d, i.e., hVR1d.1 and hVR1d.2. The derived protein, i.e., amino acid, sequences encoded by the hVR1d.1 and hVR1d.2 splice variants are depicted in FIGS. 2A and 2B, respectively.

### EXAMPLE 2

### Expression Profiling of the Novel Human hVR1d.1 and hVR1d.2 Polypeptides

Expression profiling studies utilizing the hVR1d nucleic acid sequences described above were carried out as follows: PCR primers were designed from the BAC sequences identified supra was used to measure tissue levels of hVR1d mRNA by quantitative PCR using Applied Biosystems' GeneAmp 5700. The forward primer was TGACCTGAACATCCAGCAGA (SEQ ID NO:24) and the reverse primer was AGCATGTTGAGGAGGAGAACA (SEQ ID NO:25). The primers did not distinguish between hVR1d.1 and hVR1d.2. In the PCR procedure, first strand cDNA was made from commercially available mRNA isolated from various tissue sources (CLONTECH). In addition, the relative amount of cDNA used in each assay was determined by performing a parallel experiment using a primer pair for cyclophilin, a gene expressed in equal amounts in all tissues. The cyclophilin primer pair detected small variations in the amount of cDNA in each sample and these data were used for normalization of the data obtained with the hVR1d primer pair. The PCR data was converted into a relative assessment of the difference in transcript abundance amongst the tissues tested and the data is presented in FIG. 4.

As depicted in FIG. 4, hVR1d is highly expressed in various brain tissues as well as spinal cord tissue. With regard to the brain tissues, hVR1d is most highly expressed in the corpus callosum (CC), caudate nucleus (CN), and amygdala (A) of the brain.

Moreover, additional expression profiling experiments were performed to identify the relative expression of the hVR1d splice variant, hVR1d.2, nucleic acid in various tissues, including brain subregions. The experiments were performed as described above using the primer pair that follows. The forward primer was CGGAAACCTCGGTGTAGAAG (SEQ ID NO:26) and the reverse primer was TCATCCCTCAAAGCCTCTCT (SEQ ID NO:27).

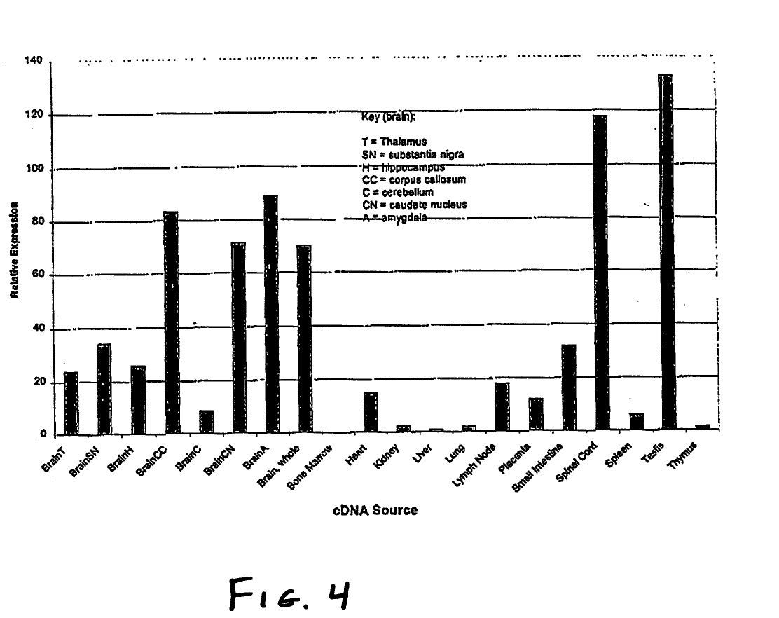

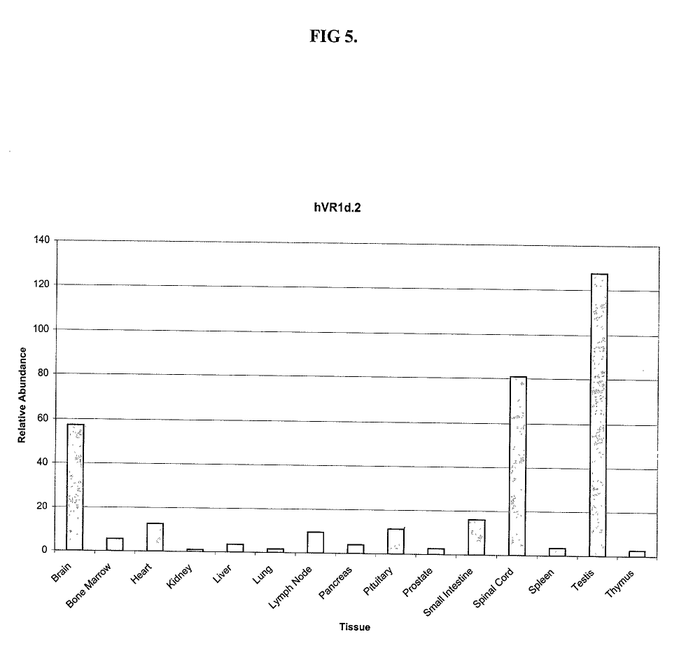

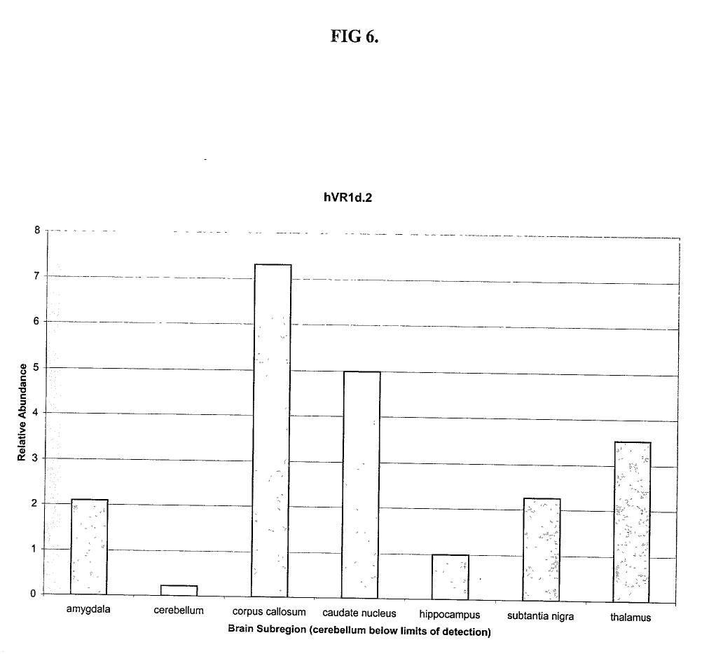

As shown in FIG. 5, the hVR1d.2 polypeptide had a very similar expression profile as the hVR1d.1 polypeptide. However, the hVR1d.2 polypeptide did show some differential expression in the brain subregions, as shown in FIG. 6. Specifically, the hVR1d.2 polypeptide was significantly more expressed in thalamus and substantia nigra, with a lower level of expression in amygdala, as compared to the hVR1d.1 polypeptide. The observed differential expression emphasizes the potentially related, yet diverse, roles of the hVR1d.1 and hVR1d.2 polypeptides, and may suggest that either one of the polypeptides may have utility as a druggable target for the treatment of different neural diseases and/or disorders.

### EXAMPLE 3

### Method of Creating N- and C-terminal Deletion Mutants Corresponding to the HVR1d.1 and hVR1d.2 Polypeptides of the Present Invention

As described elsewhere herein, the present invention encompasses the creation of N- and C-terminal deletion mutants, in addition to any combination of N- and C-terminal deletions thereof, corresponding to the HVR1d.1 and hVR1d.2 polypeptides of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length HVR1d.1 or hVR1d.2 polypeptide sequence, appropriate primers of about 15-25 nucleotides derived from the desired 5′ and 3′ positions of SEQ ID NO:1 or SEQ ID NO:3 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the H394 to R720 N-terminal deletion mutant, the following primers could be used to amplify a cDNA fragment corresponding to this deletion mutant:

| | | |
|---|---|---|
| 5′ | 5′-GCAGCA GCGGCCGC CACATGTTCTTTCTGTCCTTCTGC -3′ | (SEQ ID NO:28) |
| Primer | NotI | |
| 3′ | 5′-GCAGCA GTCGAC CCTCACAGCGACAGTACCTGTTCG -3′ | (SEQ ID NO:29) |
| Primer | SalI | |

For example, in the case of the M1 to N626 C-terminal deletion mutant, the following primers could be used to amplify a cDNA fragment corresponding to this deletion mutant:

| | | |
|---|---|---|
| 5′ | 5′-GCAGCA GCGGCCGC ATGAGCTTTATTTGCAGGCCACGAG -3′ | (SEQ ID NO:30) |
| Primer | NotI | |
| 3′ | 5′-GCAGCA GTCGAC GTTGAGGAGGAGAACAAAGGTGAGG -3′ | (SEQ ID NO:31) |
| Primer | SalI | |

Representative PCR amplification conditions are provided below, although the skilled artisan would appreciate that other conditions may be required for efficient amplification. A 100 ul PCR reaction mixture may be prepared using 10 ng of the template DNA (cDNA clone of HVR1d.1 and hVR1d.2), 200 uM 4dNTPs, 1 uM primers, 0.25 U Taq DNA polymerase (PE), and standard Taq DNA polymerase buffer. Typical PCR cycling condition are as follows:

| | |
|---|---|
| 20-25 cycles: | 45 sec, 93 degrees |
| | 2 min, 50 degrees |
| | 2 min, 72 degrees |
| 1 cycle: | 10 min, 72 degrees |

After the final extension step of PCR, 5 U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). . The skilled artisan would appreciate that other plasmids could be equally substituted, and may be desirable in certain circumstances. The digested fragment and vector are then ligated using a DNA ligase, and then used to transform competent E.coli cells using methods provided herein and/or otherwise known in the art.

The 5′ primer sequence for amplifying any additional N-terminal deletion mutants may be determined by reference to the following formula:

(S+(X*3)) to ((S+(X*3))+25), wherein ‘S’ is equal to the nucleotide position of the initiating start codon of the HVR1d.1 or hVR1d.2 gene (SEQ ID NO:1 or SEQ ID NO:3), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:1 or SEQ ID NO:3. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25), wherein ‘S’ is equal to the nucleotide position of the initiating start codon of the HVR1d.1 or hVR1d.2 gene (SEQ ID NO:1 or SEQ ID NO:3), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1 or SEQ ID NO:3. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

### EXAMPLE 4

### Method of Enhancing the Biological Activity/Functional Characteristics of Invention Through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered ion channel may be constitutively active upon binding of its cognate ligand. Alternatively, an engineered ion channel may be constitutively active in the absence of ligand binding. In yet another example, an engineered ion channel may be capable of being activated with less than all of the regulatory factors and/or conditions typically required for ion channel activation (e.g., ligand binding, phosphorylation, conformational changes, calcium flux, etc.). Such ion channel would be useful in screens to identify ion channel modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as descibed by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments—further diversifying the potential hybridation sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatman) or could be purified using Microcon concentrators (Amicon) of the appropriate molecular weight cuttoff, or could use oligonucleotide purification columns (Qiagen), in addition to other methods known in the art. If using DE81 ion-exchange paper, the 10-50 bp fragments could be eluted from said paper using 1 M NaCL, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris•HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60s; 94 C for 30s, 50-55 C for 30s, and 72 C for 30s using 30-45 cycles, followed by 72 C for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C for 30 s, 50 C for 30 s, and 72 C for 30 s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailered to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host, particularly if the polynucleotides and polypeptides provide a therapeutic use. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel varient that provided the desired characteristics.

Likewise, the invention encompasses the application of DNA shuffling technology to the evolution of polynucletotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homolog sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. The forgoing are hereby incorporated in their entirety herein for all purposes.

### EXAMPLE 5

### Method of Assessing the Putative Ion Channel Activity of the hVR1d Polypeptides

A number of methods may be employed to assess the potential ion channel activity of the hVR1d polypeptides. One preferred method is described below

CHO-K1 cells transfected with a suitable mammalian expression vector comprising the hVR1d encoding polynucleotide sequence is prepared using methods known in the art. The transfected cells are transferred to cover slips 12 hours after transfection, and electrophysiological measurements are made 24 hours after transfection (22±2° C.). The hVR1d-expressing CHO-K1 cells are detected by GFP fluorescence. Membrane currents are digitized at 10 or 20 kHz and digitally filtered off line at 1 kHz. Voltage stimuli lasting 500 ms are delivered at 5-s intervals, with either voltage ramps or voltage steps from 100 to ±100 mV. The internal pipette solution for macroscopic and single-channel currents may contain 145 mM Cs-methanesulfonate, 8 mM NaCl, 5 mM ATP, 1 mM MgCl2, 10 mM EGTA, 4.1 mM CaCl2, and 10 mM Hepes, with pH adjusted to 7.2 with CsOH after addition of ATP. The standard extracellular solution may contain 140 mM NaCl, 5 mM CsCl, 2.8 mM KCl, 2 mM CaCl2, 1 mM MgCl2, 10 mM Hepes, and 10 mM glucose, with pH adjusted to 7.4 with NaOH. Relative ion permeabilities may be measured with the pipette solution containing 145 mM Cs-methanesulfonate, 10 mM CsCl, 5 mM ATP, 10 mM EGTA, and 10 mM Hepes (pH 7.2) and the external solution containing 110 mM NMDG+, 30 mM X+ (Na+, Ca2+, K+, or Cs+), 10 mM Hepes, and 10 mM glucose (pH 7.4). The relative permeability for monovalent ions may be calculated according to the equation PX/PCs=([Cs+]o/[X+]o)exp[F(EX ECs)/RT]. The PCa/PCs permeability ratio is calculated according to the equation PCa/PCs={[Cs+]oexp(FECs/RT)exp(FECa/RT)[exp(FECa/RT)+1]}/(4[Ca2+]o), where R, T, and F are the gas constant, absolute temperature, and Faraday's constant, respectively. Statistical comparisons are made with the two-way analysis of ariance (ANOVA) and two-tailed t test with Bonferroni correction; P<0.05 indicated statistical significance.

### EXAMPLE 6

### Bacterial Expression of a Polypeptide

A polynucleotide encoding a polypeptide of the present invention is amplified using PCR oligonucleotide primers corresponding to the 5′ and 3′ ends of the DNA sequence, to synthesize insertion fragments. The primers used to amplify the cDNA insert should preferably contain restriction sites, such as BAMHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000 Xg). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6 M-1 M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified protein is stored at 4 degree C. or frozen at −80 degree C.

### EXAMPLE 7

### Purification of a Polypeptide from an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit eight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000 xg for 15 min. The resultant pellet is washed again using 0.5 M NaCl, 100 mM Tris, 50 mM EDTA, pH 7.4.

The resulting washed inclusion bodies are solubilized with 1.5 M guanidine hydrochloride (GuHCl) for 2-4 hours. After 7000 xg centrifugation for 15 min., the pellet is discarded and the polypeptide containing supernatant is incubated at 4 degree C. overnight to allow further GuHCl extraction.

Following high speed centrifugation (30,000 xg) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Commassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### EXAMPLE 8

### Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified sing PCR oligonucleotide primers corresponding to the 5′ and 3′ ends of the DNA sequence, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using PCR protocol. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized bacuolvirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and 5 ug of the plasmid are mixed in a sterile well of a microtiter plate containing 50 ul of serum-free Grace's medium (Life Technologoes Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### EXAMPLE 9

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### EXAMPLE 10

### Protein Fusions

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example described herein; see also EP A 394,827; Traunecker, et al., Nature 331:84-86 (1988).) Similarly, fusion to IgG-1, IgG-3, and albumin increases the halflife time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector. Note that the polynucleotide is cloned without a stop codon, otherwise a fusion protein will not be produced.

The naturally occurring signal sequence may be used to produce the protein (if applicable). Alternatively, if the naturally occurring signal sequence is not used, the vector can be modified to include a heterologous signal sequence. (See, e.g., WO 96/34891 and/or US Pat. No. 6,066,781, supra.)

Human IgG Fc region:

| | |
|---|---|
| GGGATCCGGAGCCCAAATCTTCTGACAAAACTCACA | (SEQ ID NO:32) |
| CATGCCCACCGTGCCCAGCACCTGAATTCGAGGGTG | |
| CACCGTCAGTCTTCCTCTTCCCCCCAAAACCCAAGG | |
| ACACCCTCATGATCTCCCGGACTCCTGAGGTCACAT | |
| GCGTGGTGGTGGACGTAAGCCACGAAGACCCTGAGG | |
| TCAAGTTCAACTGGTACGTGGACGGCGTGGAGGTGC | |
| ATAATGCCAAGACAAAGCCGCGGGAGGAGCAGTACA | |
| ACAGCACGTACCGTGTGGTCAGCGTCCTCACCGTCC | |
| TGCACCAGGACTGGCTGAATGGCAAGGAGTACAAGT | |
| GCAAGGTCTCCAACAAAGCCCTCCCAACCCCCATCG | |
| AGAAAACCATCTCCAAAGCCAAAGGGCAGCCCCGAG | |
| AACCACAGGTGTACACCCTGCCCCCATCCCGGGATG | |
| AGCTGACCAAGAACCAGGTCAGCCTGACCTGCCTGG | |
| TCAAAGGCTTCTATCCAAGCGACATCGCCGTGGAGT | |
| GGGAGAGCAATGGGCAGCCGGAGAACAACTACAAGA | |
| CCACGCCTCCCGTGCTGGACTCCGACGGCTCCTTCT | |
| TCCTCTACAGCAAGCTCACCGTGGACAAGAGCAGGT | |
| GGCAGCAGGGGAACGTCTTCTCATGCTCCGTGATGC | |
| ATGAGGCTCTGCACAACCACTACACGCAGAAGAGCC | |
| TCTCCCTGTCTCCGGGTAAATGAGTGCGACGGCCGC | |
| GACTCTAGAGGAT | |

### EXAMPLE 11

### Production of an Antibody from a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

We claim:

1. An isolated nucleic acid comprising a member of the group consisting of:
(a) a nucleic acid sequence that encodes a polypeptide having the amino acid sequence of FIG. 2A or FIG. 2B (SEQ ID NO.2 or 4);
(b) An isolated nucleic acid comprising a nucleic acid sequence capable of hybridizing under stringent conditions to a nucleic acid molecule of claim 1 and encoding a hVR1d polypeptide having an activity of a naturally-occurring hVR1d protein;
(c) An isolated nucleic acid comprising the nucleic acid sequence of FIG. 1A;
(d) An isolated nucleic acid comprising the nucleic acid sequence of FIG. 1B;
(e) An isolated polynucleotide having the nucleic acid sequence of ATCC Accession No. ______;
(f) An isolated polynucleotide having the nucleic acid sequence according to nucleotides 4 to 2160 of SEQ ID NO:1, wherein said nucleotides encode a polypeptide of SEQ ID NO:2 minus the start codon;
(g) An isolated polynucleotide having the nucleic acid sequence according to nucleotides 1 to 2160 of SEQ ID NO:1, wherein said nucleotides encode a polypeptide of SEQ ID NO:2 including the start codon;
(h) An isolated polynucleotide having the nucleic acid sequence according to nucleotides 4 to 2235 of SEQ ID NO:3, wherein said nucleotides encode a polypeptide of SEQ ID NO:4 minus the start codon;
(i) An isolated polynucleotide having the nucleic acid sequence according to nucleotides 1 to 2235 of SEQ ID NO:3, wherein said nucleotides encode a polypeptide of SEQ ID NO:4 including the start codon;
(j) the complement of the nucleic acid sequence of any one of (a) thru (i);
(k) An isolated nucleic acid wherein a nucleic acid of any one of (a) thru (j) that encodes an hVR1d protein or polypeptide is linked in frame to a nucleic acid sequence that encodes a heterologous protein or peptide;
(l) A nucleic acid comprising a nucleic acid sequence encoding (a) a deletion mutant of hVR1d.1; (b) a deletion mutant of hVR1d.2; or (c) the complement of the nucleic acid sequences of (a) or (b);
(m) A nucleic acid comprising a nucleic acid sequence encoding (a) a substitution mutant of hVR1d.1; (b) a substitution mutant of hVR1d.2; or (c) the complement of the nucleic acid sequences of (a) or (b);

2. A recombinant vector comprising a nucleic acid of claim 1.

3. An expression vector comprising a nucleic acid of claim 1 operatively associated with a regulatory nucleotide sequence containing transcriptional and translational regulatory information that controls expression of the nucleic acid in a host cell.

4. A genetically engineered host cell containing a nucleic acid of claim 1.

5. A genetically engineered host cell containing a nucleic acid of claim 1 operatively associated with a regulatory nucleotide sequence containing transcriptional and translational regulatory information that controls expression of the nucleic acid sequence in a host cell.

6. A method of making an hVR1d polypeptide comprising the steps of:
(a) culturing the host cell of claim 4 in an appropriate culture medium to produce an hVR1d polypeptide; and
(b) isolating the hVR1d polypeptide.

7. A method of making an hVR1d polypeptide comprising the steps of:
(a) culturing the host cell of claim 5 in an appropriate culture medium to produce an hVR1d polypeptide; and
(b) isolating the hVR1d polypeptide.

8. The method of claim 6 or 7, wherein the hVR1d polypeptide is hVR1d1.1 or hVR1d.2 or a functionally equivalent derivative thereof.

9. An antibody preparation which is specifically reactive with an epitope of an hVR1d polypeptide.

10. A transgenic animal comprising a nucleic acid of claim 1.

11. A substantially pure polypeptide encoded by a nucleic acid of claim 1.

12. A substantially pure human hVR1d polypeptide as depicted in FIGS. 2A or 2B (SEQ ID NO: 2 or 4).

13. A substantially pure polypeptide which is at least 90% identical to the polypeptide as set forth in FIGS. 2A or 2B (SEQ ID NO: 2 or 4).

14. A fusion protein comprising a polypeptide of claim 13 and a second heterologous polypeptide.

15. A pharmaceutical preparation comprising a therapeutically effective amount of the polypeptide of claim 11 and a pharmaceutically acceptable carrier.

16. A test kit for detecting and/or quantitating a wild type or mutant hVR1d nucleic acid molecule in a sample, comprising the steps of contacting the sample with a nucleic acid of claim 1; and detecting and/or quantitating the label as an indication of the presence or absence and/or amount of a wild type or mutant hVR1d nucleic acid.

17. A method for identifying compounds that modulate hVR1d activity comprising:
(a) contacting a test compound to a cell that expresses a hVR1d gene;
(b) measuring the level of hVR1d gene expression in the cell; and
(c) comparing the level obtained in (b) with the hVR1d gene expression obtained in the absence of the compound;
such that if the level obtained in (b) differs from that obtained in the absence of the compound, a compound that modulates hVR1d activity is identified.

18. A method for identifying compounds that regulate ion channel-related disorders, comprising:
(a) contacting a test compound with a cell which expresses a nucleic acid of claim 1 and
(b) determining whether the test compound modulates hVR1d activity.

19. A method for identifying compounds that regulate ion channel-related disorders, comprising:
(a) contacting a test compound with a cell or cell lysate containing a reporter gene operatively associated with a hVR1d regulatory element; and
(b) detecting expression of the reporter gene product.

20. A method for identifying compounds that regulate ion channel-related disorders comprising:
(a) contacting a test compound with a cell or cell lysate containing hVR1d transcripts; and
(b) detecting the translation of the hVR1d transcript.

21. A method for modulating ion channel-related disorders in a subject, comprising administering to the subject a therapeutically effective amount of a hVR1d polypeptide.

22. A method for the treatment of ion channel-related disorders, comprising modulating the activity of a hVR1d polypeptide.

23. The method of claim 22, wherein the method comprises administering an effective amount of a compound that agonizes or antagonizes the activity of the hVR1d polypeptide.

24. A method for the treatment of ion channel-related disorders, comprising administering an effective amount of a compound that decreases expression of a hVR1d gene.

25.) A method of identifying a compound that modulates the biological activity of hVR1d, comprising:
(a) combining a candidate modulator compound with hVR1d having the sequence set forth in SEQ ID NO:2; and
(b) measuring an effect of the candidate modulator compound on the activity of hVR1d.

26.) A compound that modulates the biological activity of human hVR1d as identified by the method according to claim 25.
